SEQUENCE LISTING (120 sequences)

SEQ ID NO: 1-17 - skipped (17 entries)

SEQ ID NO: 18 - DNA, 86 bp, synthetic construct
  source 1..86: mol_type = other DNA
gtttaagagctatgctggaaacagcatagcaagtttaaataaggctagtccgttatcaacttgaaaaagtggcaccgagtcggtgc

SEQ ID NO: 19 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 20 - protein, 7 aa, synthetic construct
PKKKRKV

SEQ ID NO: 21 - protein, 5 aa, synthetic construct
  REPEAT 1..5: note = repeat unit: repeats 0 to 10 times
GGGGS

SEQ ID NO: 22 - protein, 5 aa, synthetic construct
GGGGG

SEQ ID NO: 23 - protein, 5 aa, synthetic construct
GGAGG

SEQ ID NO: 24 - protein, 7 aa, synthetic construct
GGGGSSS

SEQ ID NO: 25 - protein, 7 aa, synthetic construct
GGGGAAA

SEQ ID NO: 26 - protein, 1368 aa, Streptococcus pyogenes [sequence omitted]

SEQ ID NO: 27 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 28 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - DNA, 3159 bp, synthetic construct
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 31 - DNA, 3159 bp, synthetic construct
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 32 - DNA, 4107 bp, synthetic construct
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 33 - DNA, 3158 bp, synthetic construct
  source 1..3158: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 34 - DNA, 3159 bp, synthetic construct
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 35 - DNA, 3159 bp, synthetic construct
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 36 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 37 - DNA, 3242 bp, synthetic construct
  source 1..3242: mol_type = other DNA
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 38 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 39 - DNA, 3156 bp, synthetic construct
  source 1..3156: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 40 - DNA, 7009 bp, synthetic construct
  source 1..7009: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 41 - protein, 2414 aa, synthetic construct
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 42 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 43 - protein, 1497 aa, synthetic construct
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 44 - DNA, 4491 bp, synthetic construct
  source 1..4491: mol_type = other DNA
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 45 - protein, 65 aa, synthetic construct
RTLVTFKDVFVDFTREEWKLLDTAQQILYRNVMLENYKNLVSLGYQLTKPDVILRLEKGEEPWLV

SEQ ID NO: 46 - DNA, 195 bp, synthetic construct
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 47 - protein, 1519 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 48 - DNA, 4560 bp, synthetic construct
  source 1..4560: mol_type = other DNA
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 49 - protein, 1190 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 50 - DNA, 3570 bp, synthetic construct
  source 1..3570: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 51 - protein, 718 aa, Synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 52 - DNA, 2154 bp, synthetic construct
  source 1..2154: mol_type = other DNA
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 53 - protein, 378 aa, Synthetic construct
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 54 - DNA, 1134 bp, Synthetic construct
  source 1..1134: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 55 - protein, 522 aa, Synthetic construct
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - DNA, 1566 bp, Synthetic construct
  source 1..1566: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 57 - DNA, 21 bp, Synthetic construct
  source 1..21: mol_type = other DNA
ccggtggccttattataaagg

SEQ ID NO: 58 - DNA, 21 bp, Synthetic construct
  source 1..21: mol_type = other DNA
tccagcgtgtgagcctgggag

SEQ ID NO: 59 - DNA, 21 bp, Synthetic construct
  source 1..21: mol_type = other DNA
acatatctgggaaaagaccta

SEQ ID NO: 60 - DNA, 21 bp, Synthetic construct
  source 1..21: mol_type = other DNA
tccgcccatgtgacttccagc

SEQ ID NO: 61 - DNA, 21 bp, Synthetic construct
  source 1..21: mol_type = other DNA
gtcacatgggcggaaacttca

SEQ ID NO: 62 - DNA, 21 bp, Synthetic construct
  source 1..21: mol_type = other DNA
cggggacactgggccgacgcg

SEQ ID NO: 63 - DNA, 21 bp, Synthetic construct
  source 1..21: mol_type = other DNA
ctggaggagcgagagtggcgc

SEQ ID NO: 64 - DNA, 21 bp, Synthetic construct
  source 1..21: mol_type = other DNA
cctgcgtccgccccccggcgc

SEQ ID NO: 65 - DNA, 21 bp, Synthetic construct
  source 1..21: mol_type = other DNA
cttcggggccgctggaggagc

SEQ ID NO: 66 - DNA, 21 bp, Synthetic construct
  source 1..21: mol_type = other DNA
aggcttctaggtaaactaagg

SEQ ID NO: 67 - DNA, 21 bp, Synthetic construct
  source 1..21: mol_type = other DNA
attaagatggtttattacagg

SEQ ID NO: 68 - DNA, 21 bp, Synthetic construct
  source 1..21: mol_type = other DNA
gctccagagactgatgaatgg

SEQ ID NO: 69 - DNA, 21 bp, Synthetic construct
  source 1..21: mol_type = other DNA
ccggctccccgttacctgtgc

SEQ ID NO: 70 - DNA, 21 bp, Synthetic construct
  source 1..21: mol_type = other DNA
cagttgaggcttgaagggcca

SEQ ID NO: 71 - DNA, 21 bp, Synthetic construct
  source 1..21: mol_type = other DNA
ctagccaccagggagctacgg

SEQ ID NO: 72 - DNA, 21 bp, Synthetic construct
  source 1..21: mol_type = other DNA
gaaactgggaggaaggcgcgg

SEQ ID NO: 73 - DNA, 21 bp, Synthetic construct
  source 1..21: mol_type = other DNA
gcacattcctttcccagaagg

SEQ ID NO: 74 - DNA, 21 bp, Synthetic construct
  source 1..21: mol_type = other DNA
gtttcaaggaggggaaaaaga

SEQ ID NO: 75 - DNA, 21 bp, Synthetic construct
  source 1..21: mol_type = other DNA
gggcggagctcatgtcgagga

SEQ ID NO: 76 - DNA, 21 bp, Synthetic construct
  source 1..21: mol_type = other DNA
cagacttagccgaggacgagg

SEQ ID NO: 77 - DNA, 21 bp, Synthetic construct
  source 1..21: mol_type = other DNA
ccgagacgaaaaatgaagcca

SEQ ID NO: 78 - DNA, 21 bp, Synthetic construct
  source 1..21: mol_type = other DNA
ccatcccgggatggatggagg

SEQ ID NO: 79 - DNA, 21 bp, Synthetic construct
  source 1..21: mol_type = other DNA
agctcgggctggataagggct

SEQ ID NO: 80 - DNA, 21 bp, Synthetic construct
  source 1..21: mol_type = other DNA
ttcccccaccaccagcggcga

SEQ ID NO: 81 - DNA, 21 bp, Synthetic construct
  source 1..21: mol_type = other DNA
tgttttgcagacggaaaatgc

SEQ ID NO: 82 - DNA, 21 bp, Synthetic construct
  source 1..21: mol_type = other DNA
gtgatggggagaaacggggca

SEQ ID NO: 83 - DNA, 21 bp, Synthetic construct
  source 1..21: mol_type = other DNA
ggcggaggctacgaaacttgg

SEQ ID NO: 84 - DNA, 21 bp, Synthetic construct
  source 1..21: mol_type = other DNA
ggtgcagtgtgaggcgcagga

SEQ ID NO: 85 - DNA, 21 bp, Synthetic construct
  source 1..21: mol_type = other DNA
taaagttattgtgaatgggga

SEQ ID NO: 86 - DNA, 21 bp, Synthetic construct
  source 1..21: mol_type = other DNA
gccggggaggcgaagcggcgg

SEQ ID NO: 87 - DNA, 21 bp, Synthetic construct
  source 1..21: mol_type = other DNA
tcctgcccgcgagcacgggac

SEQ ID NO: 88 - DNA, 21 bp, Synthetic construct
  source 1..21: mol_type = other DNA
gagagggctgcagaaccctgg

SEQ ID NO: 89 - RNA, 21 nt, Synthetic construct
  source 1..21: mol_type = other RNA
ccggtggccttattataaagg

SEQ ID NO: 90 - RNA, 21 nt, Synthetic construct
  source 1..21: mol_type = other RNA
tccagcgtgtgagcctgggag

SEQ ID NO: 91 - RNA, 21 nt, Synthetic construct
  source 1..21: mol_type = other RNA
acatatctgggaaaagaccta

SEQ ID NO: 92 - RNA, 21 nt, Synthetic construct
  source 1..21: mol_type = other RNA
tccgcccatgtgacttccagc

SEQ ID NO: 93 - RNA, 21 nt, Synthetic construct
  source 1..21: mol_type = other RNA
gtcacatgggcggaaacttca

SEQ ID NO: 94 - RNA, 21 nt, Synthetic construct
  source 1..21: mol_type = other RNA
cggggacactgggccgacgcg

SEQ ID NO: 95 - RNA, 21 nt, Synthetic construct
  source 1..21: mol_type = other RNA
ctggaggagcgagagtggcgc

SEQ ID NO: 96 - RNA, 21 nt, Synthetic construct
  source 1..21: mol_type = other RNA
cctgcgtccgccccccggcgc

SEQ ID NO: 97 - RNA, 21 nt, Synthetic construct
  source 1..21: mol_type = other RNA
cttcggggccgctggaggagc

SEQ ID NO: 98 - RNA, 21 nt, Synthetic construct
  source 1..21: mol_type = other RNA
aggcttctaggtaaactaagg

SEQ ID NO: 99 - RNA, 21 nt, Synthetic construct
  source 1..21: mol_type = other RNA
attaagatggtttattacagg

SEQ ID NO: 100 - RNA, 21 nt, Synthetic construct
  source 1..21: mol_type = other RNA
gctccagagactgatgaatgg

SEQ ID NO: 101 - RNA, 21 nt, Synthetic construct
  source 1..21: mol_type = other RNA
ccggctccccgttacctgtgc

SEQ ID NO: 102 - RNA, 21 nt, Synthetic construct
  source 1..21: mol_type = other RNA
cagttgaggcttgaagggcca

SEQ ID NO: 103 - RNA, 21 nt, Synthetic construct
  source 1..21: mol_type = other RNA
ctagccaccagggagctacgg

SEQ ID NO: 104 - RNA, 21 nt, Synthetic construct
  source 1..21: mol_type = other RNA
gaaactgggaggaaggcgcgg

SEQ ID NO: 105 - RNA, 21 nt, Synthetic construct
  source 1..21: mol_type = other RNA
gcacattcctttcccagaagg

SEQ ID NO: 106 - RNA, 21 nt, Synthetic construct
  source 1..21: mol_type = other RNA
gtttcaaggaggggaaaaaga

SEQ ID NO: 107 - RNA, 21 nt, Synthetic construct
  source 1..21: mol_type = other RNA
gggcggagctcatgtcgagga

SEQ ID NO: 108 - RNA, 21 nt, Synthetic construct
  source 1..21: mol_type = other RNA
cagacttagccgaggacgagg

SEQ ID NO: 109 - RNA, 21 nt, Synthetic construct
  source 1..21: mol_type = other RNA
ccgagacgaaaaatgaagcca

SEQ ID NO: 110 - RNA, 21 nt, Synthetic construct
  source 1..21: mol_type = other RNA
ccatcccgggatggatggagg

SEQ ID NO: 111 - RNA, 21 nt, Synthetic construct
  source 1..21: mol_type = other RNA
agctcgggctggataagggct

SEQ ID NO: 112 - RNA, 21 nt, Synthetic construct
  source 1..21: mol_type = other RNA
ttcccccaccaccagcggcga

SEQ ID NO: 113 - RNA, 21 nt, Synthetic construct
  source 1..21: mol_type = other RNA
tgttttgcagacggaaaatgc

SEQ ID NO: 114 - RNA, 21 nt, Synthetic construct
  source 1..21: mol_type = other RNA
gtgatggggagaaacggggca

SEQ ID NO: 115 - RNA, 21 nt, Synthetic construct
  source 1..21: mol_type = other RNA
ggcggaggctacgaaacttgg

SEQ ID NO: 116 - RNA, 21 nt, Synthetic construct
  source 1..21: mol_type = other RNA
ggtgcagtgtgaggcgcagga

SEQ ID NO: 117 - RNA, 21 nt, Synthetic construct
  source 1..21: mol_type = other RNA
taaagttattgtgaatgggga

SEQ ID NO: 118 - RNA, 21 nt, Synthetic construct
  source 1..21: mol_type = other RNA
gccggggaggcgaagcggcgg

SEQ ID NO: 119 - RNA, 21 nt, Synthetic construct
  source 1..21: mol_type = other RNA
tcctgcccgcgagcacgggac

SEQ ID NO: 120 - RNA, 21 nt, Synthetic construct
  source 1..21: mol_type = other RNA
gagagggctgcagaaccctgg

(19) United States
(12) Patent Application Publication
(10) Pub. No.: US 2025/0197823 A1
(43) Pub. Date: Jun. 19, 2025

(54) COMPOSITIONS AND METHODS FOR EPIGENOME EDITING TO ENHANCE T CELL THERAPY

(71) Applicant: Duke University, Durham, NC (US)

(72) Inventors: Charles A. Gersbach, Chapel Hill, NC (US); Sean McCutcheon, Durham, NC (US)

(21) Appl. No.: 18/840,832

(22) PCT Filed: Feb. 24, 2023
(86) PCT No.: PCT/US2023/063297

§ 371 Date: Aug. 22, 2024

Related U.S. Application Data
(60) Provisional application No. 63/426,671, filed on Nov. 18, 2022.
(60) Provisional application No. 63/330,679, filed on Apr. 13, 2022.
(60) Provisional application No. 63/325,039, filed on Mar. 29, 2022.
(60) Provisional application No. 63/314,256, filed on Feb. 25, 2022.

(51) Int. Cl. C12N 9/22; A61K 48/00; A61P 35/00; C12N 15/11; C12N 15/90

(52) U.S. Cl. CPC C12N 9/22; A61P 35/00; C12N 15/11; C12N 15/907; A61K 48/00; C12N 2310/20

(57) ABSTRACT

Disclosed herein are compositions and methods for modulating T cells. For example, the compositions and methods may be used to increase memory T cells. The compositions and method may be used in combination with Adoptive T Cell Therapy (ACT) to enhance the ACT.

### CROSS-REFERENCE TO RELATED APPLICATIONS

This application claims priority to U.S. Provisional Patent Application No. 63/314,256, filed Feb. 25, 2022, U.S. Provisional Patent Application No. 63/325,039, filed Mar. 29, 2022, U.S. Provisional Patent Application No. 63/330,679, filed Apr. 13, 2022, and U.S. Provisional Patent Application No. 63/426,671, filed Nov. 18, 2022, the entire contents of each of which are hereby incorporated by reference.

### FIELD

This disclosure relates to CRISPR-Cas systems and their use in screening for gene targets to increase T cells such as memory T cells, as well as compositions and methods targeting the genes to improve adoptive T cell therapy (ACT).

### INTRODUCTION

Adoptive T cell therapy (ACT) harnesses the immune system to target and kill cancer cells. Although ACT can produce major durable responses, many patients either relapse or are completely refractory to ACT. Several ACT trials across diverse cancer types have revealed that positive responses are closely linked with specific CD8+ T cell subsets in the engineered T cell product. Memory T cells were markedly enriched in the T cell product of responders, indicating their unique capacity to self-renew, proliferate, and persist led to pronounced tumor killing. However, a major concern is that cancer patients' T cell repertoires are often skewed away from memory T cells and towards exhausted (T_{EX}) or effector T cells (T_{EFF}) because of chronic antigen stimulation at the tumor site. Moreover, epigenetic signatures of exhaustion persist even after checkpoint blockage and antigen clearance as T cells acquire a fixed chromatin landscape. Epigenetic reprogramming of T_{EX} and T_{EFF} has been widely postulated as a promising strategy to improve ACT, but there is limited understanding of which genes and epigenetic programs to manipulate to efficiently reprogram T cells for effective ACT.

### SUMMARY

In an aspect, the disclosure relates to a composition for modulating T cells. The composition may include a modulator of a gene selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and FLI1. In some embodiments, modulating T cells comprises increasing T cells, or increasing memory T cells, or increasing the lifetime of a T cell, or preventing T cell exhaustions, or reversing T cell exhaustions, or reducing T cell exhaustion, or enhancing the therapeutic potential of T cells, or a combination thereof. In some embodiments, the modulator comprises a polypeptide, or a polynucleotide, or a small molecule, or a lipid, or a carbohydrate, or a combination thereof. In some embodiments, the modulator comprises an antibody or siRNA or shRNA. In some embodiments, the modulator comprises a DNA targeting composition, the DNA targeting composition comprising: a Cas9 protein or a fusion protein, wherein the fusion protein comprises two heterologous polypeptide domains, wherein the first polypeptide domain comprises a Cas9 protein and the second polypeptide domain has an activity selected from transcription activation activity, transcription repression activity, nuclease activity, transcription release factor activity, histone modification activity, nucleic acid association activity, methylase activity, and demethylase activity; and at least one guide RNA (gRNA) that targets the Cas9 protein to the gene or a regulatory element thereof.

In a further aspect, the disclosure relates to a DNA targeting composition comprising: a Cas9 protein or a fusion protein, wherein the fusion protein comprises two heterologous polypeptide domains, wherein the first polypeptide domain comprises a Cas9 protein and the second polypeptide domain has an activity selected from transcription activation activity, transcription repression activity, nuclease activity, transcription release factor activity, histone modification activity, nucleic acid association activity, methylase activity, and demethylase activity; and at least one guide RNA (gRNA) that targets the Cas9 protein to a target gene or a regulatory element thereof, wherein the target gene is selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and FL11. In some embodiments, the gRNA is encoded by a polynucleotide comprising a sequence selected from SEQ ID NOs: 57-88, or it comprises a sequence selected from SEQ ID NOs: 89-120. In some embodiments, the Cas protein comprises a Streptococcus pyogenes Cas9 protein, or a Staphylococcus aureus Cas9 protein, or any fragment thereof. In some embodiments, the Cas9 protein comprises an amino acid sequence having at least 90% or greater identity to a sequence selected from SEQ ID NOs: 26-29, or any fragment thereof, or is encoded by a polynucleotide comprising a sequence having at least 90% or greater identity to a sequence selected from SEQ ID NOs: 30-39, or any fragment thereof. In some embodiments, the Cas9 protein comprises an amino acid sequence having one, two, three, four, five or more changes selected from amino acid substitutions, insertions, or deletions, relative to a sequence selected from SEQ ID NOs: 26-29, or any fragment thereof, or is encoded by a polynucleotide comprising a sequence having one, two, three, four, five or more changes selected from nucleotide substitutions, insertions, or deletions, relative to a sequence selected from SEQ ID NOs: 30-39, or any fragment thereof. In some embodiments, the Cas9 protein comprises the amino acid sequence of one of SEQ ID NOs: 26-29, or any fragment thereof, or is encoded by a polynucleotide comprising a sequence selected from SEQ ID NOs: 30-39. In some embodiments, the fusion protein comprises more than one second polypeptide domain. In some embodiments, the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, MxiI repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3. In some embodiments, the second polypeptide domain has transcription repression activity. In some embodiments, the second polypeptide domain comprises KRAB. In some embodiments, KRAB comprises an amino acid sequence having at least 90% or greater identity to SEQ ID NO: 45, or any fragment thereof, or is encoded by a polynucleotide comprising a sequence having at least 90% or greater identity to SEQ ID NO: 46, or any fragment thereof. In some embodiments, KRAB comprises an amino acid sequence having one, two, three, four, five or more changes selected from amino acid substitutions, insertions, or deletions, relative to SEQ ID NO: 45, or any fragment thereof, or is encoded by a polynucleotide comprising a sequence having one, two, three, four, five or more changes selected from nucleotide substitutions, insertions, or deletions, relative to SEQ ID NO: 46, or any fragment thereof. In some embodiments, KRAB comprises the amino acid sequence of SEQ ID NO: 45, or any fragment thereof, or is encoded by a polynucleotide comprising the sequence of SEQ ID NO: 46. In some embodiments, the fusion protein comprises an amino acid sequence having at least 90% or greater identity to SEQ ID NO: 47 or 49, or any fragment thereof, or is encoded by a polynucleotide comprising a sequence having at least 90% or greater identity to SEQ ID NO: 48 or 50, or any fragment thereof. In some embodiments, the fusion protein comprises an amino acid sequence having one, two, three, four, five or more changes selected from amino acid substitutions, insertions, or deletions, relative to SEQ ID NO: 47 or 49, or any fragment thereof, or is encoded by a polynucleotide having one, two, three, four, five or more changes selected from nucleotide substitutions, insertions, or deletions, relative to SEQ ID NO: 48 or 50. In some embodiments, the fusion protein comprises the amino acid sequence of SEQ ID NO: 47 or 49, or any fragment thereof, or is encoded by a polynucleotide comprising the sequence of SEQ ID NO: 48 or 50. In some embodiments, the second polypeptide domain has transcription activation activity. In some embodiments, the second polypeptide domain comprises a polypeptide selected from VP16, VP64, p65, TET1, VPR, VPH, Rta, and p300, or a fragment thereof. In some embodiments, the second polypeptide domain comprises VP64, p300, VPH, or VPR, or a fragment thereof. In some embodiments, the second polypeptide domain comprises an amino acid sequence having at least 90% or greater identity to SEQ ID NO: 41, 42, 53, or 55, or any fragment thereof, or is encoded by a polynucleotide comprising a sequence having at least 90% or greater identity to SEQ ID NO: 54 or 56, or any fragment thereof. In some embodiments, the second polypeptide domain comprises an amino acid sequence having one, two, three, four, five or more changes selected from amino acid substitutions, insertions, or deletions, relative to SEQ ID NO: 41, 42, 53, or 55, or any fragment thereof, or is encoded by a polynucleotide comprising a sequence having one, two, three, four, five or more changes selected from nucleotide substitutions, insertions, or deletions, relative to SEQ ID NO: 54 or 56, or any fragment thereof. In some embodiments, the second polypeptide domain comprises the amino acid sequence of SEQ ID NO: 41, 42, 53, or 55, or any fragment thereof, or is encoded by a polynucleotide comprising the sequence of SEQ ID NO: 54 or 56. In some embodiments, the fusion protein comprises an amino acid sequence having at least 90% or greater identity to SEQ ID NO: 43, or any fragment thereof, or is encoded by a polynucleotide comprising a sequence having at least 90% or greater identity to SEQ ID NO: 44, or any fragment thereof. In some embodiments, the fusion protein comprises an amino acid sequence having one, two, three, four, five or more changes selected from amino acid substitutions, insertions, or deletions, relative to SEQ ID NO: 43, or any fragment thereof, or is encoded by a polynucleotide comprising a sequence having one, two, three, four, five or more changes selected from nucleotide substitutions, insertions, or deletions, relative to SEQ ID NO: 44. In some embodiments, the fusion protein comprises the amino acid sequence of SEQ ID NO: 43, or any fragment thereof, or is encoded by a polynucleotide comprising the sequence of SEQ ID NO: 44.

Another aspect of the disclosure provides a composition for increasing T cells. The composition may include an activator of a gene selected from BATF3, EOMES, NR1 D1, and JUN. In some embodiments, the activator comprises a polynucleotide encoding the gene.

Another aspect of the disclosure provides a composition for increasing T cells, the composition comprising an inhibitor of a gene selected from BATF, DNMT1, FOXO1, MYB, and BACH2. In some embodiments, the inhibitor comprises a shRNA or siRNA targeting the gene or a fragment thereof.

Another aspect of the disclosure provides a composition for increasing T cells, wherein the composition comprises an activator of the BATF3 gene. In some embodiments, the activator comprises a polynucleotide encoding BATF3. In some embodiments, the activator comprises the DNA targeting composition as detailed herein, wherein the second polypeptide domain has transcription activation activity. In some embodiments, the gene is BATF3 and the gRNA targets the Cas9 protein to or is encoded by a polynucleotide sequence comprising a sequence selected from SEQ ID NOs: 62-65, or the gene is EOMES and the gRNA targets the Cas9 protein to or is encoded by a polynucleotide sequence comprising a sequence selected from SEQ ID NOs: 57-58, or wherein the gene is NR1D1 and the gRNA targets the Cas9 protein to or is encoded by a polynucleotide sequence comprising the sequence of SEQ ID NO: 82, or the gene is JUN and the gRNA targets the Cas9 protein to or is encoded by a polynucleotide sequence comprising the sequence of SEQ ID NO: 79. In some embodiments, the gene is BATF3 and the gRNA comprises a polynucleotide sequence selected from SEQ ID NOs: 94-97, or the gene is EOMES and the gRNA comprises a polynucleotide sequence selected from SEQ ID NOs: 89-90, or the gene is NR1D1 and the gRNA comprises the polynucleotide sequence of SEQ ID NO: 114, or the gene is JUN and the gRNA comprises the polynucleotide sequence of SEQ ID NO: 111. In some embodiments, the inhibitor comprises the DNA targeting composition as detailed herein, wherein the second polypeptide domain has transcription repression activity. In some embodiments, the gene is BATF and the gRNA targets the Cas9 protein to or is encoded by a polynucleotide sequence comprising a sequence selected from SEQ ID NOs: 59-61, or the gene is DNMT1 and the gRNA targets the Cas9 protein to or is encoded by a polynucleotide sequence comprising the sequence of SEQ ID NO: 71, or the gene is FOXO1 and the gRNA targets the Cas9 protein to or is encoded by a polynucleotide sequence comprising the sequence of SEQ ID NO: 72, or the gene is MYB and the gRNA targets the Cas9 protein to or is encoded by a polynucleotide sequence comprising a sequence selected from SEQ ID NOs: 68-69, or the gene is BACH2 and the and the gRNA targets the Cas9 protein to or is encoded by a polynucleotide sequence comprising the sequence of SEQ ID NO: 85. In some embodiments, the gene is BATF and the gRNA comprises a polynucleotide sequence selected from SEQ ID NOs: 91-93, or the gene is DNMT1 and the gRNA comprises the polynucleotide sequence of SEQ ID NO: 103, or the gene is FOXO1 and the gRNA comprises the polynucleotide sequence of SEQ ID NO: 104, or the gene is MYB and the gRNA comprises a polynucleotide sequence selected from SEQ ID NOs: 100-101, or the gene is BACH2 and the gRNA comprises the polynucleotide sequence of SEQ ID NO: 117. In some embodiments, the composition further includes at least one cancer therapy.

Another aspect of the disclosure provides an isolated polynucleotide sequence encoding the composition as detailed herein.

Another aspect of the disclosure provides a vector comprising an isolated polynucleotide sequence as detailed herein.

Another aspect of the disclosure provides a cell comprising a composition as detailed herein, or an isolated polynucleotide sequence as detailed herein, or a vector as detailed herein, or a combination thereof. In some embodiments, the cell is a CD8+ T cell.

Another aspect of the disclosure provides a pharmaceutical composition comprising a composition as detailed herein, or an isolated polynucleotide sequence as detailed herein, or a vector as detailed herein, or a combination thereof.

Another aspect of the disclosure provides a method of modulating T cells. The method may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof. In some embodiments, modulating T cells comprises increasing T cells, or increasing memory T cells, or preventing T cell exhaustions, or reversing T cell exhaustions, or a combination thereof.

Another aspect of the disclosure provides a method of increasing T cells. The method may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof.

Another aspect of the disclosure provides a method of enhancing adoptive T cell therapy (ACT) in a subject. The method may include administering to the subject a composition as detailed herein, or an isolated polynucleotide sequence as detailed herein, or a vector as detailed herein, or a cell as detailed herein, or a pharmaceutical composition as detailed herein, or a combination thereof.

Another aspect of the disclosure provides a method of treating cancer in a subject. The method may include administering to the subject a composition as detailed herein, or an isolated polynucleotide sequence as detailed herein, or a vector as detailed herein, or a cell as detailed herein, or a pharmaceutical composition as detailed herein, or a combination thereof.

The disclosure provides for other aspects and embodiments that will be apparent in light of the following detailed description and accompanying figures.

### BRIEF DESCRIPTION OF THE DRAWINGS

FIGS. 1A-1B. Volcano plots of CRISPR activation (a) and interference (i) transcription factor (TF) screens in primary human T cells. CD8+CCR7+ T cells were transduced with either an (FIG. 1A) all-in-one repressor (dSaCas9-KRAB) lentiviral vector for the CRISPRi screen or (FIG. 1B) activator (dSaCas9-2xVP64) lentiviral vector for the CRISPRa screen with a pool of 2,099 unique gRNAs. Each point represents an individual gRNA. The y-axis corresponds to statistical significance of gRNA representation between CCR7− and CCR7+ bins, and the x-axis indicates the log 2 fold change of gRNA counts between bins. gRNAs with an adjusted p value <0.05 are labeled and annotated with their target gene. Light gray points indicate non-targeting gRNAs. Several gene targets had more than one enriched gRNA, including EOMES (2 gRNAs), BATF3 (3 gRNAs), BATF (3 gRNAs), and CREM (2 gRNAs). BATF3 and BATF had opposite effects in CRISPRi/a screens.

FIGS. 2A-2B. Validation of CRISPRi-mediated effects of DNMT1 and FOXO1 silencing. (FIG. 2A) Shown is a graph of immunophenotyping of memory markers (IL7RA, left and CD27, right) on T cells treated with non-targeting (NT), DNMT1, or FOXO1 gRNAs. Each unique shade of gray corresponds to a specific donor. (FIG. 2B) Shown is a graph of gene expression of DNMT1 or FOXO1. RT-qPCR was used to confirm silencing of DNMT1 and FOXO1 at a transcriptional level. The ddCT method was used to calculate the fold change with normalization to GAPDH and target gene expression levels in NT samples. DNTM1 and FOXO1 silencing affected several memory associated surface markers.

FIGS. 3A-3B. Validation of CRISPRa-mediated effects of BATF3 activation. (FIG. 3A) Shown is immunophenotyping of CCR7 on T cells treated with GFP or BATF3-2A-GFP encoding lentivirus (left panel) and non-targeting or BATF3-targeting gRNA (right panel). Lines are drawn to indicate shared donors. (FIG. 3B) Shown is a graph of gene expression for BATF3. RT-qPCR was used to confirm activation of BATF3 at a transcriptional level. The ddCT method was used to calculate the fold change with normalization to GAPDH and BATF3 expression in NT samples. CRISPRa mediated BATF3 upregulation and BATF3 ORF overexpression had similar effects on CCR7 expression.

FIG. 4. Effects of BATF3 ORF overexpression on IL7RA expression over time. Shown is a graph of IL7RA expression (%). T cells were treated with GFP or BATF3-2A-GFP encoding lentivirus and stained for IL7RA at days 5, 8, and 12. BATF3 open reading frame overexpression led to dramatic increase in IL7RA, which is a marker of T cell persistence.

FIG. 5. Global effects of BATF3 captured through RNA-sequencing (RNA-seq). Shown is a MA plot with log 2fold shrinkage after DESeq2 analysis comparing gene counts between BATF3-treated cells and GFP-treated cells. BATF3 induced widespread changes in gene expression. There were 506 upregulated genes in BATF3 treated cells. There were 520 downregulated genes in BATF3 treated cells. Statistical threshold: Padj<0.05.

FIG. 6. Overlap between core exhausted gene set (90 genes) and 323 differentially downregulated genes in BATF3-treated T cells. Shown is a diagram indicating 16/90 (>17%) of exhausted genes were downregulated in BATF3-treated T cells. BATF3 attenuated T cell exhaustion programs.

FIG. 7. BATF3 overexpression co-silences external and internal checkpoint inhibitors: TIGIT, LAG3, TIM3, and CISH. Shown is a graph of expression values for each gene in transcripts per million (TPM) plotted across untreated, GFP, and BATF3-treated T cells. Individual points represent expression for a given donor. All four genes have a P_{adj}<0.01 when comparing counts between GFP and BATF3 samples. BATF3 overexpression co-silenced external and internal checkpoint inhibitors, which are clinically relevant targets.

FIGS. 8A-8C. Shown is a graph of expression values for each gene in transcripts per million (TPM) plotted across untreated, GFP, and BATF3-treated T cells. The expression profiles of 19 overlapping exhausted genes are grouped by expression level: (FIG. 8A) low, (FIG. 8B) medium, and (FIG. 8C) high.

FIG. 9. Targeted TF gRNA design. Shown is a graph of the number of TFs versus the number of gRNAs. The mean number of gRNAs per TF was 16.5.

FIG. 10. Schematic diagram of the CRISPR TF v2.0 screens to find transcriptional and epigenetic regulators of T cell state.

FIG. 11 is a table of the validated high confidence TF gRNA hits across CRISPRi/a screens.

FIGS. 12A-12E are figures showing CRISPRa TF validation for EOMES (FIG. 12A), BATF (FIG. 12B), CREM (FIG. 12C), and BATF3 (FIG. 12D), with the change in CCR7 shown for all in FIG. 12E.

FIGS. 13A-13D are figures showing CRISPRi TF validation for DNMT1 and FOXO1 (FIG. 13A), and that more subtle effects might be masked in individual validations (FIG. 13B and FIG. 13C). scRNA-seq was used to capture global effects of gRNA perturbations (FIG. 13D). Most TF hits may have widespread gene expression changes.

FIG. 14 is a schematic diagram showing the BATF3 open reading frame (ORF) lentiviral construct.

FIG. 15A-15D. FIG. 15A and FIG. 15B show BATF3 ORF overexpression mimics the effects of CRISPR activation. FIG. 15C and FIG. 15D show that BATF3 overexpression increases IL7RA levels.

FIG. 16A-16B. FIG. 16A shows the results of gene ontology analyses, revealing the global effects of BATF3. FIG. 16B shows that BATF3 induces rapid-recall gene expression programs.

FIG. 17 shows that BATF3 restrains effector/exhaustion programs, BATF3 increases expression of other critical regulators, and BATF3 supports bioenergic demands of memory T cells.

FIGS. 18A-18K. Development of compact and efficient dSaCas9 epigenome editors. FIG. 18A is a schematic of all-in-one lentiviral construct encoding for a gRNA cassette and dSaCas9^{KRAB} linked to a reporter (GFP or Thy1.1) via a 2A polypeptide skipping sequence. FIG. 18B is a schematic of CD2 and B2M CRISPRi screens in human CD8+ T cells from pooled PBMC donors. CD8+ T cells were transduced with either the CD2 (n=2 replicates) or B2M (n=3 replicates) CRISPRi gRNA library. Cells were expanded for 9-10 days and then stained for the target gene (CD2 or B2M). Transduced cells in the bottom and top 10% tails of CCR7 expression were sorted for gRNA library preparation and sequencing. FIG. 18C is a volcano plot of significance (P_{adj}) versus fold-change in gRNA abundance based on differential analysis between CD2-high and CD2-low populations for the CD2 CRISPRi screen. Blue data points indicate CD2 gRNA hits with a P_{adj}<0.05 or log₂(fc)<−1. Black data points indicate non-significant CD2 gRNAs. Gray data points indicate the 250 non-targeting (NT) gRNAs. FIG. 18D shows the fold change in CD2 gRNA abundance based on differential DESeq2 analysis from CRISPRi screen versus gRNA position relative to the transcriptional start site (TSS). Dashed lines represent the previously defined optimal window (−50 to +300 bp of TSS) for CRISPRi. FIG. 18E shows the fold change in CD2 gRNA abundance as a function of the final base pair of the PAM (NNGRRN). x represents the number of gRNA hits for each PAM and y represents the total number of gRNAs for each PAM. A global one-way ANOVA with Dunnett's post hoc test was used to compare the average fold change of gRNAs for each PAM variant to NNGRRT (*<0.05 denotes that the fold change of gRNAs targeting NNGRRT PAMs was significantly different than all other PAM variants). FIG. 18F is the validation of CD2 regulation with positive gRNA hits from the pooled screen (n=3 replicates of CD8+ T cells from pooled PBMC donors). The percentage of CD2 positive cells was quantified using flow cytometry on day 9 post-transduction and plotted in rank order based on the mean gRNA activity (error bars represent SEM). The final base pair of the PAM for each gRNA is indicated beneath the gRNA label. FIG. 18G shows the correlation between CD2 gRNA activity as measured in individual validations and the fold change in gRNA abundance from the screen. Mean fluorescence intensity (MFI) was normalized to the MFI of NT population. Pearson's correlation coefficient (r) is indicated in the upper left. Volcano plots of significance (P_{adj}) versus fold-change in gRNA abundance based on differential DESeq2 analysis between IL2RA-high and IL2RA-low populations for the IL2RA CRISPRa screens (n=3 replicates) with (FIG. 18H) dSaCas9-VP64 and (FIG. 18I) VP64-dSaCas9-VP64. Yellow and purple data points indicate respective gRNA hits (P_{adj}<10-5). Black data points indicate non-significant IL2RA gRNAs. Gray data points indicate the 94 NT gRNAs. FIG. 18J shows the normalized IL2RA MFI of dSaCas9-VP64 and VP64-dSaCas9-VP64 Jurkat lines transduced with indicated gRNAs (n=2 replicates). IL2RA MFI was normalized to the MFI of dSaCas9^{VP64} Jurkat line transduced with NT. A paired ratio t-test was used to compare gRNA activity between dSaCas9-VP64 and VP64-dSaCas9-VP64 Jurkat lines. FIG. 18K shows the relative IL2RA mRNA expression of Jurkat CRISPRa lines transduced with indicated gRNA on day 9 post-transduction (n=2, error bars represent SEM) using RT-qPCR. Fold change in IL2RA expression was calculated using the 2^{(-ddCT)} method with normalization to GAPDH and then normalization to IL2RA expression of VP64-dSaCas9-VP64-NT group. Sp indicates a VP64-dSaCas9-VP64 Jurkat line transduced with a previously validated IL2RA gRNA (Simeonov, D. R. et al. Nature 2017, 549, 111-115). Unless otherwise indicated, a global one-way ANOVA with Dunnett's post hoc test was used to compare each gRNA hit to the NT for validation experiments.

FIGS. 19A-19E. Proof-of-principle CRISPRi screens tiling the promoters of CD2 and B2M in primary human CD8+ T cells. FIG. 19A is a volcano plot of significance (Padj) versus fold-change in gRNA abundance based on differential analysis between B2M-high and B2M-low populations for the B2M CRISPRi screen in CD8+ T cells from pooled PBMC donors (n=3 replicates). Medium gray data points indicate B2M gRNA hits with a Padj<10-10. Black data points indicate non-significant B2M gRNAs. Light gray data points indicate the 250 NT gRNAs. FIG. 19B shows the fold change in B2M gRNA abundance based on differential DESeq2 analysis from CRISPRi screen versus gRNA position relative to the transcriptional start site (TSS). Dashed lines represent the previously defined optimal window (−50 to +300 bp of TSS) for CRISPRi. FIG. 19C shows the fold change in B2M gRNA abundance as a function of the final base pair of the PAM (NNGRRN). x represents the number of gRNA hits for each PAM and y represents the total number of gRNAs for each PAM. A global one-way ANOVA with Dunnett's post hoc test was used to compare the average fold change of gRNAs for each PAM variant to NNGRRT (*<0.05 denotes that the fold change of gRNAs targeting NNGRRT PAMs was significantly different than all other PAM variants). FIG. 19D is the validation of B2M regulation with positive gRNA hits from the pooled screen (n=3 replicates of CD8+ T cells from pooled PBMC donors). The percentage of B2M positive cells was quantified using flow cytometry on day 9 post-transduction and plotted in rank order based on the mean gRNA activity (error bars represent SEM). The final base pair of the PAM for each gRNA is indicated beneath the gRNA label. FIG. 19E shows the relative B2M mRNA expression of cells transduced with indicated gRNA on day 9 post-transduction (n=3 replicates of CD8+ T cells from pooled PBMC donors, error bars represent SEM) using RT-qPCR. Fold change in B2M expression was calculated using the 2(−ddCT) method with normalization to GAPDH and then normalization to B2M expression of NT group.

FIGS. 20A-20G. Flow cytometry validation of CD2 and B2M gRNA hits and multiplex gene silencing. Shown are representative contour plots of (FIG. 20A) CD2 and (FIG. 20B) B2M expression in CD8+ T cells across non-targeting (NT), non-hit (NH), and gRNA hits (H) measured on day 9 post transduction using flow cytometry. FIG. 20C shows the correlation between relative mean fluorescence intensity (MFI) of CD2 in CD2 silenced cells and the percentage of CD2 silenced cells. MFI was normalized to the MFI of NT cell population. Pearson's correlation coefficient (r) is indicated above the graph. FIG. 20D shows the correlation between B2M gRNA activity as measured in individual validations and the fold change in gRNA abundance from the screen. Pearson's correlation coefficient (r) is indicated above the graph. Also shown is the average percentage of (FIG. 20E) CD2 silenced, (FIG. 20F) B2M silenced, and (FIG. 20G) dual CD2 and B2M silenced CD8+ T cells on day 10 post transduction with the indicated pairs of nontargeting, CD2, and B2M gRNAs (n=3 replicates of CD8+ T cells from pooled PBMC donors, error bars represent SEM). g1 is driven by a human U6 promoter and g2 is driven by a mouse U6 promoter. CD2 H8 and B2M H1 gRNAs were used for multiplex gene silencing experiments.

FIGS. 21A-21E. Multimodal single cell RNA-sequencing screen using CRISPRi gRNA library tiling CD2. FIG. 21A shows a volcano plot of significance (Padj) versus average fold-change in CD2 mRNA expression between cells assigned to each CD2 gRNA compared to non-perturbed cells. FIG. 21B are violin plots of CD2 mRNA expression across cells grouped by gRNA. Light gray data points indicate cells with non-targeting gRNAs. Dark gray data points indicate cells with the indicated CD2 gRNA. The number of cells assigned to each gRNA are listed above the respective violin plot. FIG. 21C is a volcano plot of significance (Padj) versus average fold-change in CD2 protein expression between cells assigned to each CD2 gRNA compared to non-perturbed cells. FIG. 21D are violin plots of CD2 protein expression across cells grouped by gRNA. Light gray data points indicate cells with non-targeting gRNAs. Dark gray data points indicate cells with the indicated CD2 gRNA. FIG. 21E shows the correlation between the average expression of CD2 protein and mRNA for each CD2 gRNA hit defined by the cell-sorting based screen. Pearson's correlation coefficient (r) is indicated above the graph. For this pilot scRNA-seq screen, we used a gRNA UMI>1 for gRNA to cell assignment and the negative binomial test within Suerat's FindMarkers function for differential CD2 expression analyses.

FIGS. 22A-22E. Characterization of dSaCas9-based activators. FIG. 22A shows the UCSC genome browser track of IL2RA locus with statistical significance displayed for each gRNA in VP64dSaCas9VP64 CRISPRa screen. gRNA hits are annotated and labeled in blue. ATAC-seq and ENCODE candidate cis regulatory elements (cCREs) tracks are overlayed for visualization of chromatin accessibility and annotations. cCREs in red are defined as promoter-like elements and cCREs in blue are defined as enhancer-like elements. FIG. 22B shows the fold change in gRNA abundance as a function of the final base pair of the PAM (NNGRRN) for IL2RA gRNAs. The number above each column indicates the total number of gRNAs for each PAM. A global one-way ANOVA with Dunnett's post hoc test was used to compare the average fold change of gRNAs for each PAM variant to NNGRRT (*<0.05 denotes that the fold change of gRNAs targeting NNGRRT PAMs was significantly different than all other PAM variants). FIG. 22C are representative overlayed histograms of IL2RA expression for dSaCas9VP64 and VP64dSaCas9VP64 Jurkat lines on day 9 post-transduction with indicated gRNAs. FIG. 22D are representative contour plots of EGFR expression in primary human CD8 T cells on day 8 post-transduction with all-in-one lentiviruses encoding for VP64dSaCas9VP64 and either a non-targeting (NT) or EGFR gRNA. FIG. 22E shows the summary statistics of EGFR activation (n=2 replicates of CD8+ T cells from pooled PBMC donors, error bars represent SEM). A global one-way ANOVA with Dunnett's post hoc test was used to compare the effect of EGFR gRNAs to the NT gRNA.

FIGS. 23A-23B. TF and epigenetic modifier gRNA library design. FIG. 23A is a histogram showing the distribution of the number of gRNAs targeting each of the 121 candidate genes with selected genes labeled. FIG. 23B shows the transduction rate as a function of the volume of CRISPRi or CRISPRa TF gRNA lentivirus added to CD8 T cells 24 hours post activation. Transduction rates were determined using flow cytometry on day 9 post transduction.

FIGS. 24A-24D. CRISPR interference and activation gene screens to identify transcriptional and epigenetic regulators of human CD8+ T cell state. FIG. 24A is a schematic of CRISPRi/a TF screens. CD8+CCR7+ T cells were sorted from donors and transduced with CRISPRi (n=2 individual donors) or CRISPRa (n=3 individual donors) gRNA libraries. Cells were expanded for 10 days and then stained for Thy1.1 and CCR7. Transduced cells in the bottom and top 10% tails of CCR7 expression were sorted for gRNA library preparation and sequencing. FIG. 24B shows details of TF and epigenetic modifier gRNA library design. Also shown are volcano plots of significance (P_{adj}) versus fold-change in gRNA abundance based on differential analysis between CCR7-high and CCR7-low populations for the (FIG. 24C) CRISPRi and (FIG. 24D) CRISPRa screens. Red and blue data points indicate gRNA hits (P_{adj}<0.05) that either decrease or increase CCR7 expression and each gRNA hit is annotated with its target gene. Black data points indicate non-significant gRNAs. Gray data points indicate the 120 NT gRNAs.

FIGS. 25A-25D. Quality control of differential gene expression analyses from CRISPRi and CRISPRi TF scRNA-seq characterization. Shown is the average number of differentially expressed genes (DEGs) for targeting and nontargeting gRNAs as a function of the gRNA UMI threshold for gRNA assignment to cells in (FIG. 25A) CRISPRi and (FIG. 25B) CRISPRa scRNA-seq screens. Also shown are volcano plots of (FIG. 25C) CRISPRi and (FIG. 25D) CRISPRa scRNA-seq screens with the statistical significance (Padj) of each significant gRNA-gene pair plotted versus the fold change in gene expression relative to non-perturbed cells.

FIGS. 26A-26F. Single cell RNA-sequencing characterization of gene candidates. Shown are volcano plots of significance (P_{adj}) versus average fold change of CCR7 expression for each gRNA compared to non-perturbed cells for (FIG. 26A) CRISPRi and (FIG. 26B) CRISPRa perturbations. Red and blue data points indicate gRNA hits (P_{adj}<0.05) that decrease or increase CCR7 expression. Gray data points indicate NT gRNAs. FIG. 26C is a dot plot depicting the average expression and percent of cells expressing target genes, memory markers, and effector molecules for the indicated CRISPRi perturbations. FIG. 26D is a scatter plot of the number of differentially expressed genes (DEGs defined as P_{adj}<0.01) associated with each gRNA versus the gRNA effect on the target gene for both CRISPRi and CRISPRa perturbations. Also shown are representative enriched pathways for the top three (FIG. 26E) CRISPRi and (FIG. 26F) CRISPRa gRNAs.

FIGS. 27A-27H. Correlation between gRNAs targeting the same gene. FIG. 27A shows the correlation between union set of DEGs for CRISPRi MYB g8 versus MYB g5. FIG. 27B are violin plots of MYB expression across cells assigned to indicated gRNA with the fold change in target gene expression relative to NT and the percent of cells expressing the target gene indicated above. FIG. 27C shows the correlation between union set of DEGs for CRISPRa BATF3 g18 versus BATF3 g2. FIG. 27D are violin plots of BATF3 expression across cells assigned to indicated gRNA with the fold change in target gene expression relative to NT and the percent of cells expressing the target gene indicated above. FIG. 27E shows the correlation between union set of DEGs for CRISPRa CREM g7 versus CREM g1. FIG. 27F are violin plots of CREM expression across cells assigned to indicated gRNA with the fold change in target gene expression relative to NT and the percent of cells expressing the target gene indicated above. FIG. 27G shows the correlation between union set of DEGs for CRISPRa EOMES g11 versus EOMES g8. Only example of discordance between gRNAs targeting the same gene. Explained by the fact that EOMES g8 does not upregulate EOMES. FIG. 27H are violin plots of EOMES expression across cells assigned to indicated gRNA with the fold change in target gene expression relative to NT and the percent of cells expressing the target gene indicated above.

FIGS. 28A-28B. MYBprograms memory phenotype. FIG. 28A is a volcano plot of statistical significance (Padj) for each gene versus the fold change in gene expression in MYB CRISPRi-perturbed cells relative to non-perturbed cells. All DEGs (Padj<0.01) are labeled blue apart from MYB, which is labeled dark red. Selected DEGs are annotated. FIG. 28B shows the classification of annotated DEGs based on their functional role.

FIGS. 29A-29C. NR1D1 synthetically induces exhaustion phenotype. FIG. 29A is a UMAP plot of CRISPRa scRNA-seq characterization with cells split by perturbation status: non-perturbed (top) and perturbed (bottom). Blue data points indicate cells with a NR1 D1 gRNA. Cells were clustered using Seurat's CalcPerturbSig function to mitigate confounding sources of variation such as the donor and phase of cell cycle. FIG. 29B is a volcano plot of statistical significance (Padj) for each gene versus the fold change in gene expression in NR1D1 CRISPRa-perturbed cells relative to non-perturbed cells. All DEGs (Padj<0.01) are labeled blue apart from NR1 D1, which is labeled dark red. Selected DEGs are annotated. FIG. 29C is a violin plot of exhaustion gene signature score across all non-perturbed and NR1 D1-perturbed cells in the CRISPRa scRNA-seq screen. UCell gene signature scores are based on the Mann-Whitney U statistic.

FIGS. 30A-30G. BATF3 overexpression promotes memory T cell gene expression programs. FIG. 30A is a representative histogram of IL7R expression in CD8+ T cells with or without BATF3 overexpression measured on day 8 post transduction using flow cytometry. FIG. 30B shows summary statistics of the effect of BATF3 overexpression on IL7R expression (n=3 individual donors, paired t test conducted to determine statistical significance between treatments, lines connect the same donor). FIG. 30C shows RNA-seq comparing transcriptomes of CD8+ T cells with or without BATF3 overexpression on day 10 post transduction. Dark gray data points indicate differentially expressed genes (DEGs, P_{adj}<0.05, n=3 independent donors). MA plot is presented with log fold change shrinkage using the Bayesian shrinkage estimator ‘apeglm.’ FIG. 30D shows selected enriched and (FIG. 30E) depleted biological pathways from BATF3 overexpression. FIG. 30F is a heatmap of DEGs related to T cell exhaustion, activation, effector function as well as transcriptional or epigenetic activity and glycolysis. FIG. 30G is a volcano plot of significance (P_{adj}) versus fold-change between BATF3 and control CD8+ T cells for a subset of 136 genes that were negatively (dark gray points) or positively (light gray) associated with clinical outcome to CD19 CAR T cell treatment. The size of each data point corresponds to the strength of association between gene expression and clinical response.

FIGS. 31A-31B. BATF3 programs a transcriptional response associated with positive clinical outcome to CAR T cell therapy. FIG. 31A shows expression of the top 10 upregulated DEGs in a specific CD8+CD19 CAR T cell cluster that was significantly more prevalent in the infused CAR T cell product of nonresponders than responders treated with tisagenlecleucel (tisa-cel)54. Genes were stratified into two bar plots based on their baseline expression. FIG. 31B is a schematic of comparing the transcriptomes of CD8 T cells in the CAR T cell infusion product of non-responders (NR, dark gray) and responders (R, light gray) using published scRNAseq data54.

FIGS. 32A-32E. BATF3 remodels the chromatin landscape. FIG. 32A shows the number of ATAC-seq regions with increased or decreased accessibility in CD8+ T cells (n=3 individual donors, differential accessible regions defined by P_{adj}<0.05) with BATF3 overexpression on day 14 post-transduction. FIG. 32B shows the proportion of differentially accessible (DA) regions within each specified genomic feature. FIG. 32C is a joint analysis of RNA-seq and ATAC-seq datasets. Number of differentially accessible regions near upregulated and downregulated genes. Dashed lines represent the number of unique DEGs associated with DA regions. The ratio of DA regions per DEG is displayed on the right. FIG. 32D is a heatmap of DA regions with selected regions annotated with their nearest gene. FIG. 32E are representative ATAC-seq tracks at IL7R and TIGIT loci with overlayed rectangles indicating DA regions.

FIGS. 33A-33J. BATF3 counters phenotypic and epigenetic signatures of T cell exhaustion and improves in vitro and in vivo tumor killing. FIG. 33A is a schematic of a HER2 CAR T cell engaging with a HER2+ tumor cell and releasing inflammatory cytokines and cytotoxic molecules. FIG. 33B shows the average percentage of viable tumor cells after 24 hours of co-culture with GFP+CAR−, GFP+CAR+, and BATF3+CAR+CD8 T cells at indicated effector to target (E:T) cell ratios (n=3 individual donors, error bars represent SEM). A two-way ANOVA with Dunnett's post hoc test was used to compare tumor killing between GFP+CAR+ and BATF3+CAR+ T cells at each E:T ratio. FIG. 33C are representative histograms of exhaustion markers (TIGIT, LAG3, and TIM3) in acutely and chronically stimulated CD8+ T cells with or without BATF3 overexpression measured on day 12 post transduction using flow cytometry. The mean fluorescence intensity (MFI) and the percent of cells expressing each marker are displayed. FIG. 33D is a stacked bar chart with average proportion of untreated, GFP, or BATF3 CD8+ T cells expressing 0, 1, 2, or 3 exhaustion markers (TIGIT, LAG3, TIM3) on day 12 after acute or chronic stimulation (n=3 independent donors, error bars represent SEM). FIG. 33E shows the number of ATAC-seq regions with increased or decreased accessibility in HER2-CAR-2A-BATF3 CD8+ T cells compared to HER2-CAR-2A-GFP CD8+ T cells on day 14 post-transduction after repeated rounds of tumor restimulation (n=2 individual donors, differential accessible regions defined by P_{adj}<0.05). FIG. 33F shows the proportion of differentially accessible (DA) regions within each specified genomic feature. FIG. 33G is a heatmap of DA regions with selected regions annotated with their nearest gene. FIG. 33H are representative ATAC-seq tracks at IL7R, CTLA4, TIGIT, and KLF2 loci with overlayed rectangles indicating DA regions. FIG. 33I shows the average tumor volume over time for untreated mice and mice treated with 5×10⁵ HER2 CAR T cells with or without BATF3 overexpression (n=5 mice per treatment, error bars represent SEM). FIG. 33J shows the average tumor volume over time for untreated mice and mice treated with 2.5×10⁵ HER2 CAR T cells with or without BATF3 overexpression (n=4 mice per CAR treatment, error bars represent SEM). A two-way ANOVA was used to compare the tumor volumes at each time point across treatments for figures (FIG. 33I) and (FIG. 33J). The asterisks above the lower line indicate statistical significance between mice treated with HER2 CAR T cells with or without BATF3 overexpression at each time point.

FIGS. 34A-34D. BATF3 overexpression silences exhaustion-associated markers. FIG. 34A shows the average percentage of viable tumor cells after 24 hours of culture in T cell media, coculture with CAR− T cells, or co-culture with a titration of CAR+ T cell doses ranging from 1:8 to 2:1 E:T (n=3 individual donors, error bars represent SEM). FIG. 34B shows the percentage of viable tumor cells after 24 hours of co-culture with GFP+CAR−, GFP+CAR+, and BATF3+CAR+CD8 T cells at indicated effector to target (E:T) cell ratios for each donor. FIG. 34C shows the average percentage of cells expressing (top panel) and mean fluorescence intensity (MFI, bottom panel) of exhaustion markers: PD1, LAG3, TIGIT, and TIM3 on day 3 posttransduction with GFP or BATF3 (n=3 individual donors, error bars represent SEM, paired t tests used to determine statistical significance). FIG. 34D is a time course of the average percentage of cells expressing PD1, LAG3, TIGIT, and TIM3 post-transduction with GFP or BATF3. Acutely stimulated cells (dashed lines) were only stimulated initially, whereas chronically stimulated cells (solid lines) were stimulated with 3:1 ratio of CD3/CD28 dynabeads to cell every 3 days (n=3 individual donors, error bars represent SEM).

FIG. 35. Chronic antigen stimulation drives extensive chromatin remodeling in control CD8 T cells. Heatmap of differentially accessible regions with selected regions annotated with their nearest gene.

FIGS. 36A-36B. Determining sub curative dose of standard CAR T cells and the response of individual mice to treatment with CAR T cells with or without BATF3 overexpression. FIG. 36A shows the average tumor volume over time for untreated mice and mice treated with indicated doses of standard HER2 CAR T cells (error bars represent SEM). Mice were treated on day 21. FIG. 36B shows the tumor volumes for individual mice treated with 5×10⁵ (left panel) or 2.5×10⁵ (right panel) CAR T cells with or without BATF3 overexpression over time. Thinner lines represent individual mice and thicker lines represent the average tumor volume across mice in a treatment group.

### DETAILED DESCRIPTION

Provided herein are compositions and methods for increasing T cells and enhancing ACT. Transcriptions factors (TFs) are central mediators of cellular reprogramming and differentiation. As described herein, CRISPR-based epigenetic screening was used to discover gene targets that when perturbed, lead to increased memory T cells and improved cell durability and tumor control. CRISPR interference (CRISPRi) and CRISPR activation (CRISPRa) epigenome screens were leveraged to systematically screen 120 human TFs/epigenetic-modifying proteins for their role(s) in regulating T cell state and function. The gRNA library tiled a 1 kb window centered at the TSS of each gene. Using 2-3 biological donors, CD8+ T cells were initially sorted on CCR7 to discriminate early T subsets (CCR7+: naïve, central memory) from more differentiated T cell subsets (CCR7−: effector memory, effector). The CRISPRi TF gRNA library was delivered to CCR7+ T cells and the CRISPRa TF gRNA library was delivered to CCR7− T cells at an MOI=0.4. T cells were expanded in culture for 9-10 days and then stained and sorted for the lower and upper 10% bins of CCR7 expression. Genomic DNA was isolated, the gRNA cassette was amplified and deep sequenced, and gRNA counts were compared between bins using DESeq2.

Both putative positive and negative regulators of memory T cells were identified in the CRISPRi screen. Positive regulators included well-defined TFs such as FOXO1 and JUN as well as lesser characterized factors with respect to CD8 T cell differentiation, including DNMT1, NFE2L2, HMBOX1, GABPA, FOXP1, and GATA2. Canonical effector-cell associated TFs were identified, including TBX21 and EOMES. Less defined factors were identified, including RBPJ, GATA3, RFX5, and TET1. The negative regulators of memory T cells identified and detailed herein may be targeted through endogenous gene repression or gene knockout using programmable nucleases to enhance ACT.

BATF3 was identified in the CRISPRa screen with several enriched gRNAs, suggesting BATF3 can reprogram more differentiated T cell subsets back to memory T cells. BATF3 knockout in mouse CD8 T cells has been shown to substantially impair recall response to infection, but its role in human CD8 T cells is less understood. Moreover, this represents the first example of T cell reprogramming to an early differentiation state. Given the small size of BATF3 (390 bp), CD8 T cells could be reprogrammed through endogenous activation of BATF3 with epigenome activators, constitutive expression of BATF3 cDNA, or transient delivery of mRNA encoding BATF3. Collectively, the gene targets identified and described herein may be used to improve the efficacy of ACT.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Allogeneic” refers to any material derived from another subject of the same species. Allogeneic cells are genetically distinct and immunologically incompatible yet belong to the same species. Typically, “allogeneic” is used to define cells, such as stem cells, that are transplanted from a donor to a recipient of the same species.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Autologous” refers to any material derived from a subject and re-introduced to the same subject.

“Binding region” as used herein refers to the region within a target region that is recognized and bound by the CRISPR/Cas-based gene editing system.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, bladder, cervix, uterus, skin, epithelium, muscle, kidney, liver, lymph, bone, blood, ovary, prostate, lung, brain, head and neck, and/or breast. Cancer may include medulloblastoma, non-small cell lung cancer, and/or mesothelioma. In embodiments detailed herein, the cancer includes leukemia. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the leukemia is chronic myeloid leukemia (CML). In some embodiments, the leukemia is acute myeloid leukemia (AML).

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

The term “shRNA” stands for short hairpin RNA or small hairpin RNA. A shRNA is an artificial RNA molecule with a tight hairpin turn that can be used to silence target gene expression via RNA interference (RNAi). Expression of shRNA in cells may be facilitated by delivery of plasmids or viral or bacterial vectors. The shRNA is processed by Dicer into siRNA.

The term “siRNA” stands for small interfering RNA siRNA, sometimes also known as short interfering RNA or silencing RNA. A siRNA is a class of double-stranded RNA molecule. The siRNA may be natural or artificial. The siRNA forms a complex with the RNA-induced silencing complex (RISC). The antisense (guide) strand of siRNA directs RISC to mRNA that has a complementary sequence, and then the mRNA is cleaved by RISC or its translation is repressed.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment. The subject may have a disease or condition. In some embodiments, the subject has cancer. In some embodiments, the subject is human.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is a gene detailed herein as a modulator of T cells.

“Target region” as used herein refers to the region of the target gene to which the CRISPR/Cas9-based gene editing or targeting system is designed to bind.

“T cells” are a type of white blood cell of the immune system and play a central role in the adaptive immune response. T cells express a T-cell receptor (TCR) on their cell surface. The T cell receptor (TCR) of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. T cells may differentiate into different types of T cells. T cells may include, for example, CD8+ T cells (“killer T cells” or “cytotoxic T cells) and CD4+ T cells (“helper T cells”). CD8+ T cells and CD4+ T cells may further differentiate into other types of T cells including, for example, regulatory T cells (“suppressor T cells”) and memory T cells. In some embodiments herein, the T cell is a memory T cell. An antigen-naïve T cell expands and differentiates into a memory T cell after encountering the cognate antigen within the context of a major histocompatibility complex (MHC) molecule on the surface of an antigen presenting cell. Memory T cells may be CD8+ or CD4+. Memory T cells are long-lived and can quickly expand to large numbers of effector T cells upon re-exposure to their cognate antigen.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Modulators of T Cells

Provided herein are modulators of T cells. Modifying or modulating may include increasing or decreasing, for example. In some embodiments, the compositions and methods comprise an agent that increases T cells. Increasing T cells may include increasing the number of T cells and/or increasing the number of memory T cells and/or increasing the lifetime of a T cell and/or preventing T cell exhaustion and/or reducing T cell exhaustion and/or reversing T cell exhaustion and/or enhancing the therapeutic potential of T cells. In some embodiments, the compositions and methods comprise an agent that decreases expression of CCR7 and/or increases expression of IL7RA in T cells.

The agent, or the composition or the method comprising the agent, may target a gene or a regulatory element thereof. Regulatory elements include, for example, promoters and enhancers. Regulatory elements may be within 1000 base pairs of the transcription start site. Regulatory elements may be within 600 base pairs of the transcription start site. The agent, or the composition or the method comprising the agent, may modify the expression of a gene. For example, the agent, or the composition or the method comprising the agent, may reduce, inhibit, increase, or enhance the expression of a gene. The agent, or the composition or the method comprising the agent, may directly or indirectly modulate the activity of the gene's protein product. For example, the agent, or the composition or the method comprising the agent, may increase or decrease the binding or enzymatic activity of the gene's protein product, or inhibit the binding of the gene's protein product to another molecule or ligand, or increase the binding of the gene's protein product to another molecule or ligand, or increase or decrease the degradation of the gene's protein product, or a combination thereof. The targeted gene may be selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and FLI1, or a regulatory element thereof, or a region thereof. In some embodiments, the agent is an activator. As an activator, the agent may activate or enhance a gene or gene product such as the BATF3 gene to increase T cells. In some embodiments, the agent is an inhibitor, and the agent may inhibit or reduce or decrease a gene or gene product to increase T cells.

The agent may comprise, for example, a polynucleotide, a polypeptide, a small molecule, a lipid, a carbohydrate, or a combination thereof. In some embodiments, the agent comprises a protein. In some embodiments, the agent comprises an antibody. In some embodiments, the agent comprises siRNA. In some embodiments, the agent comprises a DNA targeting composition as detailed herein or at least one component thereof.

T cells may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be modulated by about 5-95%, 10-90%, 15-85%, 20-80%, or 1.5-fold to 10-fold, relative to a control. T cells may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be reduced by about 5-95%, 10-90%, 15-85%, 20-80%, or 1.5-fold to 10-fold, relative to a control. T cells may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be increased by about 5-95%, 10-90%, 15-85%, 20-80%, or 1.5-fold to 10-fold, relative to a control.

In some embodiments, the modulator of T cells is administered with a cancer therapy. The cancer therapy may include chemotherapy or immunotherapy. The cancer therapy may include adoptive T cell therapy (ACT) therapy. The cancer therapy may include a chimeric antigen receptor (CAR). A chimeric antigen receptor (CAR) may also be known as chimeric immunoreceptor, chimeric T cell receptor, or artificial T cell receptor. CARs are receptor proteins that have been engineered to give T cells the new ability to target a specific antigen. CARs are chimeric in that they may combine both antigen-binding and T cell activating functions into a single receptor. CARs may include an antigen binding domain specific for an antigen on a cancer cell. The premise of CAR-T immunotherapy is to modify T cells to recognize cancer cells in order to target and destroy them. T cells are harvested from a subject, the T cells are genetically altered to add a chimeric antigen receptor (CAR) that specifically recognizes cancer cells, and the resulting CAR-T cells may be administered to the subject to attack their tumors. In some embodiments, the modulator of T cells is administered concurrently with a cancer therapy, subsequent to a cancer therapy, or prior to a cancer therapy.

### 3. DNA Targeting Systems

A “DNA Targeting System” as used herein is a system capable of specifically targeting a particular region of DNA and modulating gene expression by binding to that region. Non-limiting examples of these systems are CRISPR-Cas-based systems, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

Each of these systems comprises a DNA-binding portion or domain, such as a guide RNA, a ZF, or a TALE, that specifically recognizes and binds to a particular target region of a target DNA. The DNA-binding portion (for example, Cas protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity, to form a fusion protein. Exemplary second polypeptide domains are detailed further below (see “Cas Fusion Protein”). For example, the DNA-binding portion can be linked to an activator and thus guide the activator to a specific target region of the target DNA. Similarly, the DNA-binding portion can be linked to a repressor and thus guide the repressor to a specific target region of the target DNA.

In some embodiments, the DNA-binding portion comprises a Cas protein, such as a Cas9 protein. Some CRISPR-Cas-based systems can operate to activate or repress expression using the Cas protein alone, not linked to an activator or repressor. For example, a nuclease-null Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor. Some CRISPR-Cas-based systems can operate to activate or repress expression using the Cas protein linked to a second protein domain, such as, for example, an activator or repressor.

### 4. CRISPR/Cas-based Gene Editing System

Provided herein are CRISPR/Cas9-based gene editing systems. The CRISPR/Cas-based gene editing system may be used to modulate T cells and/or enhance ACT. The CRISPR/Cas-based gene editing system may include a Cas protein or a fusion protein, and at least one gRNA, and may also be referred to as a “CRISPR-Cas system.”

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, and Cascade proteins. Cas12a may also be referred to as “Cpf1.” Cas12a causes a staggered cut in double stranded DNA, while Cas9 produces a blunt cut. In some embodiments, the Cas protein comprises Cas12a. In some embodiments, the Cas protein comprises Cas9. Cas9 forms a complex with the 3′ end of the sgRNA (which may be referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase Ill. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

a. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 26. In certain embodiments, the Cas9 molecule is a Staphylococcus aureus Cas9 molecule (also referred herein as “SaCas9”). SaCas9 may comprise an amino acid sequence of SEQ ID NO: 27.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G)(SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G)(SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G)(SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G)(SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12)(Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G)(SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G)(SEQ ID NO: 7), NNGRRN (R=A or G)(SEQ ID NO: 8), NNGRRT (R=A or G)(SEQ ID NO: 9), or NNGRRV (R=A or G; V=A or C or G)(SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 20).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 28. A S. pyogenes Cas9 protein with D10A and H849A mutations may comprise an amino acid sequence of SEQ ID NO: 29. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 30. In certain embodiments, the mutant S. aureus Cas9 molecule comprises a N580A mutation. The nucleotide sequence encoding this mutant S. aureus Cas9 molecule is set forth in SEQ ID NO: 31.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 32. Exemplary codon optimized nucleic acid sequences encoding a Cas9 molecule of S. aureus, and optionally containing nuclear localization sequences (NLSs), are set forth in SEQ ID NOs: 33-39. Another exemplary codon optimized nucleic acid sequence encoding a Cas9 molecule of S. aureus comprises the nucleotides 1293-4451 of SEQ ID NO: 40.

b. Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain has a different activity that what is endogenous to Cas protein. For example, the second polypeptide domain may have an activity such as transcription activation activity, transcription repression activity, transcription release factor activity, histone modification activity, nuclease activity, nucleic acid association activity, histone methylase activity, DNA methylase activity, histone demethylase activity, DNA demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. In some embodiments, the fusion protein comprises more than one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)ₙ, wherein n is an integer between 0 and 10 (SEQ ID NO: 21). In a GS linker, n can be adjusted to optimize the linker length and achieve appropriate separation of the functional domains. Other examples of linkers may include, for example, Gly-Gly-Gly-Gly-Gly (SEQ ID NO: 22), Gly-Gly-Ala-Gly-Gly (SEQ ID NO: 23), Gly/Ser rich linkers such as Gly-Gly-Gly-Gly-Ser-Ser-Ser (SEQ ID NO: 24), or Gly/Ala rich linkers such as Gly-Gly-Gly-Gly-Ala-Ala-Ala (SEQ ID NO: 25).

In some embodiments, the agent and/or Cas protein and/or the Cas fusion protein and/or gRNAs detailed herein may be used in compositions and methods for modulating expression of gene. Modulating may include, for example, increasing or enhancing expression of the gene, or reducing or inhibiting expression of the gene. The expression of the gene may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5-95%, 10-90%, 15-85%, 20-80%, or 1.5-fold to 10-fold, relative to a control. The expression of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5-95%, 10-90%, 15-85%, 20-80%, or 1.5-fold to 10-fold, relative to a control. The expression of the gene may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control.

### i) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 41 or SEQ ID NO: 42. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 43, encoded by the polynucleotide of SEQ ID NO: 44. VPH may comprise a polypeptide having the amino acid sequence of SEQ ID NO: 53, encoded by the polynucleotide of SEQ ID NO: 54. VPR may comprise a polypeptide having the amino acid sequence of SEQ ID NO: 55, encoded by the polynucleotide of SEQ ID NO: 56.

### ii) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4X repressor domain, MxiI repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, MxiI repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. KRAB may comprise a polypeptide having the amino acid sequence of SEQ ID NO: 45, encoded by a polynucleotide comprising the sequence of SEQ ID NO: 46. For example, the fusion protein may be S. pyogenes dCas9-KRAB (protein sequence comprising SEQ ID NO: 47; polynucleotide sequence comprising SEQ ID NO: 48). The fusion protein may be S. aureus dCas9-KRAB (protein sequence comprising SEQ ID NO: 49; polynucleotide sequence comprising SEQ ID NO: 50).

iii) Transcription Release Factor Activity

The second polypeptide domain can have transcription release factor activity. The second polypeptide domain can have eukaryotic release factor 1 (ERF1) activity or eukaryotic release factor 3 (ERF3) activity.

### iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 41 or SEQ ID NO: 42.

### v) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

### vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

vii) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

viii) Demethylase Activity

The second polypeptide domain can have demethylase activity. The second polypeptide domain can include an enzyme that removes methyl (CH3—) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; amino acid sequence comprising SEQ ID NO: 51; polynucleotide sequence comprising SEQ ID NO: 52). In some embodiments, the second polypeptide domain has histone demethylase activity. In some embodiments, the second polypeptide domain has DNA demethylase activity.

c. Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 (RNA), which is encoded by a sequence comprising SEQ ID NO: 18 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target the Cas9 protein or fusion protein to a gene or a regulatory element thereof. The gRNA may target the Cas protein or fusion protein to a non-open chromatin region, an open chromatin region, a transcribed region of the target gene, a region upstream of a transcription start site of the target gene, a regulatory element of the target gene, an intron of the target gene, or an exon of the target gene, or a combination thereof. In some embodiments, the gRNA targets the Cas9 protein or fusion protein to a promoter of a gene. In some embodiments, the target region is located between about 1 to about 1000 base pairs upstream of a transcription start site of a target gene. In some embodiments, the DNA targeting composition comprises two or more gRNAs, each gRNA binding to a different target region.

The gRNA may target a region of a gene that modulates T cells. The gRNA may target a region of a gene selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and FLI1, or a regulatory element thereof. In some embodiments, the gRNA targets a gene and is used in combination with a Cas9 fusion protein wherein the second polypeptide domain has transcription activation activity, to activate or enhance expression of the gene, such as the BATF3 gene, to increase T cells. In some embodiments, the gRNA targets a gene and is used in combination with a Cas9 fusion protein wherein the second polypeptide domain has transcription repression activity, to inhibit or reduce or decrease expression of the gene to increase T cells. The gRNA may comprise a polynucleotide selected from at least one of SEQ ID NOs: 89-120, or a complement thereof, or a variant thereof, or a truncation thereof. The gRNA may be encoded by a polynucleotide sequence comprising at least one of SEQ ID NOs: 57-88, or a complement thereof, or a variant thereof, or a truncation thereof. The gRNA may bind and target a polynucleotide sequence comprising at least one of SEQ ID NOs: 57-88, or a complement thereof, or a variant thereof, or a truncation thereof. A truncation may be 1, 2, 3, 4, 5, 6, 7, 8, or 9 nucleotides shorter than the sequence of any one of SEQ ID NOs: 57-120, which are shown in TABLE 1.

TABLE 1
Genes and gRNA sequences for modulating T cells.

| | | |
|---|---|---|
| Target | DNA sequence for gRNA | gRNA |
| Gene_ID | | |
| EOMES_11 | CCGGTGGCCTTATTATAAAGG | CCGGUGGCCUUAUUAUAAAGG |
| | (SEQ ID NO: 57) | (SEQ ID NO: 89) |
| EOMES_8 | TCCAGCGTGTGAGCCTGGGAG | UCCAGCGUGUGAGCCUGGGAG |
| | (SEQ ID NO: 58) | (SEQ ID NO: 90) |
| BATF_3 | ACATATCTGGGAAAAGACCTA | ACAUAUCUGGGAAAAGACCUA |
| | (SEQ ID NO: 59) | (SEQ ID NO: 91) |
| BATF_6 | TCCGCCCATGTGACTTCCAGC | UCCGCCCAUGUGACUUCCAGC |
| | (SEQ ID NO: 60) | (SEQ ID NO: 92) |
| BATF_5 | GTCACATGGGCGGAAACTTCA | GUCACAUGGGCGGAAACUUCA |
| | (SEQ ID NO: 61) | (SEQ ID NO: 93) |
| BATF3_2 | CGGGGACACTGGGCCGACGCG | CGGGGACACUGGGCCGACGCG |
| | (SEQ ID NO: 62) | (SEQ ID NO: 94) |
| BATF3_18 | CTGGAGGAGCGAGAGTGGCGC | CUGGAGGAGCGAGAGUGGCGC |
| | (SEQ ID NO: 63) | (SEQ ID NO: 95) |
| BATF3_13 | CCTGCGTCCGCCCCCCGGCGC | CCUGCGUCCGCCCCCCGGCGC |
| | (SEQ ID NO: 64) | (SEQ ID NO: 96) |
| BATF3_4 | CTTCGGGGCCGCTGGAGGAGC | CUUCGGGGCCGCUGGAGGAGC |
| | (SEQ ID NO: 65) | (SEQ ID NO: 97) |
| CREM_1 | AGGCTTCTAGGTAAACTAAGG | AGGCUUCUAGGUAAACUAAGG |
| | (SEQ ID NO: 66) | (SEQ ID NO: 98) |
| CREM_7 | ATTAAGATGGTTTATTACAGG | AUUAAGAUGGUUUAUUACAGG |
| | (SEQ ID NO: 67) | (SEQ ID NO: 99) |
| MYB_5 | GCTCCAGAGACTGATGAATGG | GCUCCAGAGACUGAUGAAUGG |
| | (SEQ ID NO: 68) | (SEQ ID NO: 100) |
| MYB_8 | CCGGCTCCCCGTTACCTGTGC | CCGGCUCCCCGUUACCUGUGC |
| | (SEQ ID NO: 69) | (SEQ ID NO: 101) |
| BHLHE40_7 | CAGTTGAGGCTTGAAGGGCCA | CAGUUGAGGCUUGAAGGGCCA |
| | (SEQ ID NO: 70) | (SEQ ID NO: 102) |
| DNMT1_1 | CTAGCCACCAGGGAGCTACGG | CUAGCCACCAGGGAGCUACGG |
| | (SEQ ID NO: 71) | (SEQ ID NO: 103) |
| FOXO1_2 | GAAACTGGGAGGAAGGCGCGG | GAAACUGGGAGGAAGGCGCGG |
| | (SEQ ID NO: 72) | (SEQ ID NO: 104) |
| NFE2L1_12 | GCACATTCCTTTCCCAGAAGG | GCACAUUCCUUUCCCAGAAGG |
| | (SEQ ID NO: 73) | (SEQ ID NO: 105) |
| GABPA_13 | GTTTCAAGGAGGGGAAAAAGA | GUUUCAAGGAGGGGAAAAAGA |
| | (SEQ ID NO: 74) | (SEQ ID NO: 106) |
| NFATC3_7 | GGGCGGAGCTCATGTCGAGGA | GGGCGGAGCUCAUGUCGAGGA |
| | (SEQ ID NO: 75) | (SEQ ID NO: 107) |
| FOXD2_4 | CAGACTTAGCCGAGGACGAGG | CAGACUUAGCCGAGGACGAGG |
| | (SEQ ID NO: 76) | (SEQ ID NO: 108) |
| POU2F1_7 | CCGAGACGAAAAATGAAGCCA | CCGAGACGAAAAAUGAAGCCA |
| | (SEQ ID NO: 77) | (SEQ ID NO: 109) |
| RREB1_6 | CCATCCCGGGATGGATGGAGG | CCAUCCCGGGAUGGAUGGAGG |
| | (SEQ ID NO: 78) | (SEQ ID NO: 110) |
| JUN_5 | AGCTCGGGCTGGATAAGGGCT | AGCUCGGGCUGGAUAAGGGCU |
| | (SEQ ID NO: 79) | (SEQ ID NO: 111) |
| ZFP1_6 | TTCCCCCACCACCAGCGGCGA | UUCCCCCACCACCAGCGGCGA |
| | (SEQ ID NO: 80) | (SEQ ID NO: 112) |
| IRF2_7 | TGTTTTGCAGACGGAAAATGC | UGUUUUGCAGACGGAAAAUGC |
| | (SEQ ID NO: 81) | (SEQ ID NO: 113) |
| NR1D1_7 | GTGATGGGGAGAAACGGGGCA | GUGAUGGGGAGAAACGGGGCA |
| | (SEQ ID NO: 82) | (SEQ ID NO: 114) |
| NR4A1_5 | GGCGGAGGCTACGAAACTTGG | GGCGGAGGCUACGAAACUUGG |
| | (SEQ ID NO: 83) | (SEQ ID NO: 115) |
| TCF7L1_9 | GGTGCAGTGTGAGGCGCAGGA | GGUGCAGUGUGAGGCGCAGGA |
| | (SEQ ID NO: 84) | (SEQ ID NO: 116) |
| BACH2_8 | TAAAGTTATTGTGAATGGGGA | UAAAGUUAUUGUGAAUGGGGA |
| | (SEQ ID NO: 85) | (SEQ ID NO: 117) |
| FLI1_9 | GCCGGGGAGGCGAAGCGGCGG | GCCGGGGAGGCGAAGCGGCGG |
| | (SEQ ID NO: 86) | (SEQ ID NO: 118) |
| HIC1_11 | TCCTGCCCGCGAGCACGGGAC | UCCUGCCCGCGAGCACGGGAC |
| | (SEQ ID NO: 87) | (SEQ ID NO: 119) |
| KLF2_11 | GAGAGGGCTGCAGAACCCTGG | GAGAGGGCUGCAGAACCCUGG |
| | (SEQ ID NO: 88) | (SEQ ID NO: 120) |

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

d. Repair Pathways

The CRISPR/Cas9-based gene editing system may be used to introduce site-specific double strand breaks at targeted genomic loci, such as a gene for modulating T cells as detailed herein. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

### i) Homology-Directed Repair (HDR)

Restoration of protein expression from a gene may involve homology-directed repair (HDR). A donor template may be administered to a cell. A donor sequence comprises a polynucleotide sequence to be inserted into a genome. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

### ii) Non-Homologous End Joining (NHEJ)

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 5. Genetic Constructs

The CRISPR/Cas9-based gene editing system may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metallothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as polynucleotide encoding a reporter protein and/or a selectable marker, such as hygromycin (“Hygro”). The reporter protein may include any protein or peptide that is suitably detectable, such as, by fluorescence, chemiluminescence, enzyme activity such as beta galactosidase or alkaline phosphatase, and/or antibody binding detection. The reporter protein may comprise a fluorescent protein. The reporter protein may comprise a protein or peptide detectable with an antibody. For example, the reporter protein may comprise green fluorescent protein (“GFP”), YFP, RFP, CFP, DsRed, luciferase, and/or Thy1.

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

### 6. Pharmaceutical Compositions

Further provided herein are pharmaceutical compositions comprising the above-described modulator of T cells or genetic constructs or gene editing systems. In some embodiments, the composition further includes at least one cancer therapy such as a chimeric antigen receptor (CAR). In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 7. Administration

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed modulator or T cells, a variety of effects may be elicited, such as, for example, T cells may be increased, T cell numbers may be increased, memory T cells may be increased, T cell exhaustion may be inhibited or prevented, T cell exhaustion may be reversed, cancer therapy may be enhanced or its effectiveness increased, or a combination thereof. Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. In some embodiments, the cell is a T cell. In some embodiments, the cell is a CD8+ T cell. In some embodiments, the cell is a CD4+ T cell.

### 8. Kits

Provided herein is a kit, which may be used to modulate, such as increase, T cells. The kit may be used in conjunction with ACT to enhance the ACT. The kit comprises genetic constructs or a composition comprising the same, as described above, and instructions for using said composition. In some embodiments, the kit comprises at least one gRNA comprising a polynucleotide sequence selected from SEQ ID NOs: 89-120, a complement thereof, a variant thereof, or fragment thereof, or gRNA encoded by a polynucleotide comprising a sequence selected from SEQ ID NOs: 57-88, a complement thereof, a variant thereof, or fragment thereof. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof for modulating T cells may include a modified AAV vector that includes a gRNA molecule(s) and a Cas9 protein or fusion protein, as described above, that specifically binds and cleaves a region of a gene selected from BATF3, FOXO1, JUN, DNMT1, NFE2L2, HMBOX1, GABPA, FOXP1, GATA2, TBX21, EOMES, RBPJ, GATA3, RFX5, and TET1, or a regulatory element thereof. 9. Methods

a. Methods of Modulating T Cells

Provided herein are methods of modulating T cells. The methods may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof. In some embodiments, modulating T cells comprises increasing T cells, or increasing memory T cells, or preventing T cell exhaustions, or reversing T cell exhaustions, or a combination thereof.

b. Methods of Increasing T Cells

Provided herein are methods of increasing T cells. The methods may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof.

c. Methods of Enhancing Adoptive T Cell Therapy (ACT)

Provided herein are methods of enhancing adoptive T cell therapy (ACT) in a subject. The methods may include administering to the subject a composition as detailed herein, or an isolated polynucleotide sequence as detailed herein, or a vector as detailed herein, or a cell as detailed herein, or a pharmaceutical composition as detailed herein, or a combination thereof.

d. Methods of Treating Cancer

Provided herein are methods of treating cancer in a subject. The methods may include administering to the subject a composition as detailed herein, or an isolated polynucleotide sequence as detailed herein, or a vector as detailed herein, or a cell as detailed herein, or a pharmaceutical composition as detailed herein, or a combination thereof.

### 10. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Initial CRISPRi/a transcription factor screens for T cell reprogramming. Transcriptions factors (TFs) are central mediators of cellular reprogramming and differentiation. CRISPR interference (CRISPRi: dSaCas9^{KRAB}) and CRISPR activation (CRISPRa: ^{VP64}dSaCas9^{VP64}) epigenome screens were leveraged to systematically screen 120 human TFs/epigenetic-modifying proteins for their role(s) in regulating T cell state and function. The TFs included TF motifs enriched in CD39-CD69-TIL compared to CD39⁺CD69⁺ in humans, TF motifs enriched in T_{STEM} (CCR7⁺PD⁻TIGIT⁻) compared to T_{PEX} (CCR7⁺PD1⁺TIGIT⁺) in humans, TF motifs enriched in peaks that close during transition from plastic to fixed exhausted state in mice, and all TF motifs associated with T cell state in mice. An additional 12 TFs were included, for a total of 121 genes. A gRNA library was designed to densely tile promoters by including all specific gRNAs within a 1 kb window centered at the TSS of each gene (+500 to −500 bp). The gRNAs adhered strictly to a PAM of NNGRRT. All gRNAs with more than 3 bp mismatches were removed from the set. gRNAs were found for all TFs except PBX2. The gRNA library contained 2,099 gRNAs with on average 16 gRNAs per gene (minimum of 7 and maximum of 33 gRNAs per TF) and 120 non-targeting gRNAs as negative controls. Using peripheral blood from 2-3 biological donors, CD8⁺ T cells were initially sorted on CCR7 to discriminate early T subsets (CCR7⁺: naïve, central memory) from more differentiated T cell subsets (CCR7+: effector memory, effector). Sorted T cells were stimulated with CD3/CD28 activation dynabeads at a 3:1 bead to cell ratio in PRIME-XV media supplemented with 5% platelet lysate, 1% penicillin/streptomycin, and 100 units/mL hIL-2. CD8⁺CCR7⁺ T cells were then transduced with either the CRISPRi or CRISPRa TF gRNA library at 24 hours post-activation. T cells were expanded in culture for 10 days and then stained and sorted into the lower and upper 10% bins of CCR7 expression after gating for transduced Thy1.1+ cells. Genomic DNA was isolated from sorted cells, the deep sequenced the gRNAs were amplified, and gRNA counts were compared between paired low and high bins using DESeq2.

Initial validation of individual CRISPRi/a gRNAs and BATF3 overexpression. CD8⁺CCR7⁺ T cells from three PBMC donors were sorted and activated with CD3/28 dynabeads as done in the screens. The next day, 50,000-100,000 cells per well were plated in a 96 well plate and transduced with 10 μL of 50× concentrated lentivirus encoding respective gRNAs or open reading frames (ORFs). The cells were cultured for 9-10 days before staining the CRISPR-treated cells with CCR7, IL7RA, CD27, and Thy1.1 antibodies and the ORF-treated cells with CD8, CCR7, and IL7RA antibodies for flow cytometry. Then 150,000-250,000 Thy1.1+ cells were sorted for RT-qPCR for individual gRNA validation samples, and 400,000 CD8⁺GFP⁺ T cells were sorted for ORF overexpression samples. RNA was isolated according to Norgen's (Norgen Biotek Corp., Ontario, Canada) total RNA purification plus kit protocol, and cDNA was generated using Thermo's Vilo Superscript (Thermo Fisher Scientific, Waltham, MA). The ddCT quantification method was used to analyze qPCR data with normalization to GAPDH and the respective target gene in non-targeting gRNA samples. To analyze RNA-seq data, the reads were first trimmed using Trimmomatic v0.32 to remove adapters and then reads were aligned to GRCh38 using STAR aligner (Langmead et al., 2009). Gene counts were obtained with featureCounts from the subread package (version 1.4.6-p4) using the comprehensive gene annotation in Gencode v22. Differential expression analysis was determined with DESeq2 where gene counts were fitted into negative binomial generalized linear models (GLMs) and Wald statistics determined significant hits.

Plasmids. All plasmids used were cloned using Gibson assembly (NEB). The all-in-one HER2 CAR constructs used for in vivo tumor control studies were cloned by digesting an empty lentiviral vector for constitutive gene expression (Addgene 79121) with MIuI and amplifying the HER2-CAR and 2A-GFP or 2A-BATF3 (gblock, IDT) fragments with appropriate overhangs for Gibson assembly. The following plasmids were deposited to Addgene: pLV hU6-gRNA hUbC-dSaCas9-KRAB-T2A-Thy1.1 (Addgene 194278) and pLV hU6-gRNA hUbC-VP64-dSaCas9-VP64-T2A-Thy1.1 (Addgene 194279).

Cell Lines. HEK293 Ts and SKBR3 s were maintained in DMEM GlutaMAX supplemented with 10% fetal bovine serum (FBS), 1 mM sodium pyruvate, 1×MEM non-essential amino acids (NEAA), 10 mM HEPES, 100 U mL⁻¹ of penicillin, and 100 μg mL-1 streptomycin. Jurkats lines were maintained in RPMI supplemented with 10% FBS, 100 U mL⁻¹ of penicillin, and 100 μg mL⁻¹ streptomycin. HCC1954 s were maintained in DMEM/F12 supplemented with 10% FBS, 100 U mL⁻¹ of penicillin, and 100 μg mL⁻¹ streptomycin.

Isolation and Culture of Primary Human T Cells. Human CD8+ T cells were obtained from either pooled PBMC donors (ZenBio) using negative selection human CD8 isolation kits (StemCell Technologies) or directly from vials containing isolated CD8+ T cells from individual donors (StemCell Technologies). For all technology development experiments, T cells were cultured in Advanced RPMI (Thermo Fisher) supplemented with 10% FBS, 100 U mL⁻¹ of penicillin and 100 μg mL⁻¹ streptomycin. For all T cell reprogramming experiments, T cells were cultured in PRIME-XV T cell Expansion XSFM (FujiFilm) supplemented with 5% human platelet lysate (Compass Biomed), 100 U mL⁻¹ of penicillin, and 100 μg mL⁻¹ streptomycin. All media was supplemented with 100 U mL⁻¹ human IL-2 (Peprotech). T cells were activated with a 3:1 ratio of CD3/CD28 dynabeads to T cells and split or expanded every 2 days to maintain T cells at a concentration of 1-2×10⁶ per mL.

Lentivirus Generation and Transduction of Primary Human T Cells. For all technology development experiments, lentivirus was produced as previously described (Black, J. B. et al. Cell reports 2020, 33, 108460). For all T cell reprogramming experiments, a recently optimized protocol for high-titer lentivirus was used (Schmidt, R. et al. Science (New York, N.Y.) 2022, 375). Briefly, 1.2×10⁶ or 7×10⁶ HEK293T cells were plated in a 6 well plate or 10 cm dish in the afternoon with 2 mL or 12 mL of complete opti-MEM (Opti-MEM™ I Reduced Serum Medium supplemented with 1× Glutamax, 5% FBS, 1 mM Sodium Pyruvate, and 1×MEM Non-Essential Amino Acids). The next morning, HEK293T cells were transfected with 0.5 μg pMD2.G, 1.5 μg psPAX2, and 0.5 μg transgene for 6 well transfections or 3.25 μg pMD2.G, 9.75 μg psPAX2, and 4.3 μg transgene for 10 cm dishes using Lipofectamine 3000. Media was exchanged 6 hours after transfection and lentiviral supernatant was collected and pooled at 24 hours and 48 hours after transfection. Lentiviral supernatant was centrifuged at 600×g for 10 min to remove cellular debris and concentrated to 50-100× the initial concentration using Lenti-X Concentrator (Takara Bio). T cells were transduced at 5-10% v/v of concentrated lentivirus at 24 hours post-activation. For dual transduction experiments, T cells were serially transduced at 24 hours and 48 hours post activation.

Design of CD2, B2M, and IL2RA gRNA Libraries. Saturation CD2 and B2M CRISPRi gRNA libraries were designed to tile a 1.05 kb bp window (−400 bp to 650 bp) around the TSS of each target gene using CRISPick (Sanson, K. R. et al. Nature Communications 2018, 9, 5416). The IL2RA CRISPRa gRNA library was designed to tile a 5 kb bp window (−4,000 bp to 1000 bp) around the TSS of IL2RA using ChopChop (Labun, K. et al. Nucleic Acids Research 2019, 47, 171-174). Any gRNA that aligned to another genomic site with fewer than four mismatches was removed from the library. Each gRNA library was designed to target dSaCas9's less restrictive PAM: NNGRRN. Respective non-targeting gRNAs were generated for each library to match the nucleotide composition of the targeting gRNAs.

gRNA Library Cloning. Oligonucleotide gRNA pools containing variable protospacer sequences and constant regions for PCR amplification were synthesized by Twist Bioscience. 2-4 ng of each oligonucleotide pool was input into a 7-cycle PCR with 2× Q5 mastermix and 10 μM of each amplification primer with the following cycling conditions: 98° C. for 10 s, 65° C. for 30 s, and 72° C. for 15 s. The gRNA amplicon was gel extracted and then PCR purified. The purified gRNA amplicon was input into a 20 μL Gibson reaction at a 5:1 insert to backbone molar ratio with 200 ng of either all-in-one CRISPRi or CRISPRa backbones digested with Esp3I, dephosphorylated using QuickCIP, and 1×SPRI-selected. The Gibson reactions were ethanol precipitated overnight and transformed into Lucigen's Endura ElectroCompetent Cells. Cloned gRNA libraries were purified for lentivirus production by midi-prepping 100 mL of bacterial culture.

CD2 and B2M CRISPRi Screens in Primary Human T Cells. CD8+ T cells from pooled PBMC donors were transduced with all-in-one lentivirus encoding for dSaCas9-KRAB-2A-GFP and either CD2 (n=2 replicates) or B2M (n=3 replicates) gRNA libraries. Cells were expanded for 9 days and then stained for the target gene (CD2 or B2M). Transduced GFP+ T cell in the lower and upper 10% tails of target gene expression were sorted for subsequent gRNA library construction and sequencing. All replicates were maintained and sorted at a minimum of 350× coverage.

Construction of CRISPRa Jurkat Lines and IL2RA CRISPRa Screens in Jurkats. Polyclonal dSaCas9-VP64 and VP64-dSaCas9-VP64 Jurkat cell lines were generated by transducing 2×10⁶ Jurkats with 2% v/v of 50× lentivirus encoding for either dSaCas9-VP64-2A-PuroR or VP64-dSaCas9-VP64-2A-PuroR. Cells were selected for five days (days 3-7 post-transduction) using 0.5 μg/mL of puromycin. After puromycin selection, 1×10⁶ dSaCas9-VP64 and VP64-dSaCas9-VP64 Jurkat cells were plated and transduced in triplicate with the IL2RA gRNA library lentivirus at a multiplicity of infection (MOI=0.4). Cells were expanded for 10 days, selected for Thy1.1 using a CD90.1 Positive Selection Kit (StemCell Technologies), and then stained for Thy1.1 and IL2RA. Transduced Thy1.1+ Jurkats in the lower and upper 10% tails of IL2RA expression were sorted for subsequent gRNA library construction and sequencing. All replicates were maintained and sorted at a minimum of 500× coverage.

TF and Epi-Modifier CRISPRi/a gRNA Library Construction. Genes were selected based on motif enrichment in differentially accessible chromatin across T cell subsets (Krishna, S. et al. Science (New York, N.Y.) 2020, 370, 1328-1334; Philip, M. et al. Nature 2017, 545, 452-456; Galletti, G. et al. Nature Immunology 2020) and a unified atlas of over 300 ATAC-seq and RNA-seq experiments from 12 studies of CD8 T cells in cancer and chronic infection (Pritykin, Y. et al. Mol. Cell 2021, 81, 2477-2493 e2410). The following transcriptional and epigenetic regulators: BACH2, TOX, TOX2, PRDM1, KLF2, BM11, DNMT1, DNMT3A, DNMT3B, TET1, and TET2 were manually added to the gene list. The TSS for each gene was extracted using CRISPick and 1,000 bp windows were constructed around each TSS (−500 to +500 bp). After establishing an SaCas9 gRNA database with the strict PAM variant (NNGRRT) using guideScan (Perez, A. R. et al. Nature Biotechnology 2017), the genomic windows were input into the guidescan_guidequery function to generate the gRNA library. Any gRNA that aligned to another genomic site with fewer than four mismatches was removed from the library. The final gRNA library contained at least seven gRNAs targeting 120/121 target gene (there were no PBX2-targeting gRNAs) with an average of 16 gRNAs per gene. 120 non-targeting gRNAs were included in the library for a total of 2,099 gRNAs.

TF and Epi-Modifier CRISPRi/a gRNA Screens. CD8+CCR7+ T cells were sorted and transduced with either CRISPRi (n=2 biological donors) or CRISPRa (n=3 biological donors) TF+epi-modifier gRNA libs. Cells were expanded for 10 days and then stained for Thy1.1 (a marker to identify transduced cells) and CCR7 (a marker associated with T cell state). Transduced Thy1.1+ T cells in the lower and upper 10% tails of CCR7 expression were sorted for subsequent gRNA library construction and sequencing. All replicates were maintained and sorted at a minimum of 300× coverage.

Genomic DNA Isolation, gRNA PCR, and Sequencing gRNA Libraries. Genomic DNA was isolated from sorted cells using Qiagen's DNeasy Blood and Tissue Kit. All genomic DNA was split across 100 μL PCR reactions with Q5 2× Master Mix, up to 1 μg of genomic DNA per reaction, and forward and reverse primers. After initial amplicon denaturation at 98° C. for 30 s, gRNA libraries were amplified through 25 PCR cycles at 98° C. for 10 s, 60° C. for 30 s, and 72° C. for 20 s, followed by a final extension at 72° C. for 20 s. PCRs were pooled together for each sample and purified using double-sided SPRI selection at 0.6× and 1.8× to remove gDNA and primer dimer. Libraries were run on a High Sensitivity D1000 tape (Agilent) to confirm the expected amplicon size and quantified using Qubit's dsDNA High Sensitivity assay. Libraries were individually diluted to 2 nM, pooled together at equal volumes, and sequenced using Illumina's MiSeq Reagent Kit v2 (50 cycles) according to manufacturer's recommendations. Read 1 was 22 cycles to sequence the 21 bp protospacers, and index read 1 was 6 cycles to sequence the sample barcodes.

Processing gRNA Sequencing and Enrichment Analysis for FACS-based Screens. FASTQ files were aligned to custom indexes for each gRNA library (generated from the bowtie2-build function) using Bowtie 2 (Langmead, B. & Salzberg, S. L. Nat. Methods 2012, 9, 357-359). Counts for each gRNA were extracted and used for further analysis. All enrichment analysis was done with R. Individual gRNA enrichment was determined using the DESeq2 (Langmead, B. & Salzberg, S. L. Nat. Methods 2012, 9, 357-359) package to compare gRNA abundance between high and low conditions for each screen. gRNAs were selected as hits if they met a specific statistical significance threshold (defined in figured legends).

Individual gRNA Validation Using Flow Cytometry. For CD2 and B2M g RNA validations, CD8 T cells were transduced in triplicate with each individual gRNA and followed the same timeline as the CRISPRi screens. On day 9, cells were stained with either a CD2 or B2M antibody and measured using flow cytometry. For IL2RA gRNA validations, dSaCas9-VP64 and VP64-dSaCas9-VP64 Jurkat lines were transduced with each gRNA hit and followed the same timeline as the CRISPRa screen. On day 9, cells were stained with a IL2RA antibody and measured using flow cytometry. The percentage of cells expressing the target gene or the mean fluorescence intensity (MFI) of the target gene were reported for flow cytometry data.

Flow Cytometry and Surface Marker Staining. An SH800 FACS Cell Sorter (Sony Biotechnology) was used for all cell sorting and analysis. For antibody staining of all surface markers except CCR7, cells were harvested, spun down at 300×g for 5 min, resuspended in flow buffer (1×PBS, 2 mM EDTA, 0.5% BSA) with the appropriate antibody dilutions, and incubated for 30 min at 4° C. on a rocker. Antibody staining of CCR7 was carried out for 30 min at 37° C. Cells were then washed with 1 mL of flow buffer, spun down at 300×g for 5 min, and resuspended in flow buffer for cell sorting or analysis. FMO controls were used to set appropriate gates for all flow panels.

Quantitative RT-qPCR. mRNA was isolated from transduced primary human CD8+ T cells or Jurkats using Norgen's Total RNA Purification Plus Kit. Reverse transcription was carried out by inputting an equal mass of mRNA for each sample into a 10 μL SuperScript Vilo cDNA Synthesis reaction. 2.0 μL of cDNA was used per PCR reaction with Perfecta SYBR Green Fastmix (Quanta BioSciences, 95072) using the CFX96 Real-Time PCR Detection System (Bio-Rad). All primers were designed to be highly specific using NCBI's primer blast tool and amplicon products were verified by melt curve analysis. All qRT-qPCR were presented as log₂ fold change in RNA normalized to GAPDH expression.

Characterization of TF Hits Using scRNA-seq. All 32 gRNA hits (as defined by a P_{adj}<0.05) from the CRISPRi/a screens and 8 non-targeting gRNAs were selected for scRNA-seq characterization. This 40-gRNA library was cloned into the all-in-one CRISPRi and CRISPRa lentiviral backbones. The experimental timeline for the scRNA-seq screens was identical to the cell sorting-based screens. CD8+CCR7+ T cells from three donors were transduced with CRISPRi and CRISPRa mini-TF gRNA libraries. T cells were expanded for 10 days and then stained and sorted for Thy1.1+ cells. Sorted cells were loaded into the Chromium X for a targeted recovery of 2×10⁴ cells per donor and treatment according to the Single Cell 5′-High-Throughput (HT) Reagent Kit v2 protocol (10× Genomics). SaCas9 gRNA sequences were captured by spiking in 2 μM of a custom primer into the reverse transcription master mix, as previously done for SpCas9 gRNA capture (Mimitou, E. P. et al. Nat. Methods 2019, 16, 409-412). The custom primer was designed to bind to the constant region of SaCas9's gRNA scaffold. 5′-Gene Expression (GEX) and gRNA libraries were separated using double-sided SPRI selection in the initial cDNA clean up step. 5′-GEX libraries were constructed according to manufacturer's protocol. gRNA libraries were constructed using two sequential PCRs (PCR 1: 10 cycles, PCR 2: 25 cycles). The PCR 1 product was purified using double-sided SPRI selection at 0.6× and 2×. 20% of the purified PCR 1 product was input into PCR 2. The PCR2 product was purified using double-sided SPRI selection at 0.6× and 1×. All libraries were run on a High Sensitivity D1000 tape to measure the average amplicon size and quantified using Qubit's dsDNA High Sensitivity assay. Libraries were individually diluted to 20 nM, pooled together at desired ratios, and sequenced on an Illumina NovaSeq S4 Full Flow Cell (200 cycles) with the following read allocation: Read 1=26, i7 index=10, Read 2=90.

Processing and Analyzing scRNA-seq. CellRanger v6.0.1 was used to process, demultiplex, and generate UMI counts for each transcript and gRNA per cell barcode. UMI counts tables were extracted and used for subsequent analyses in R using the Seurat (Butler, A., et al., Nature Biotechnology 2018) v4.1.0 package. Low quality cells with <200 detected genes, >20% mitochondrial reads, or <5% ribosomal reads were discarded. DoubletFinder (McGinnis, C. S et. al., Cell Systems 2019) was used to identify and remove predicted doublets. All remaining high-quality cells across donors for each treatment (CRISPRi or CRISPRa) were aggregated for further analyses. gRNAs were assigned to cells if they met the threshold (gRNA UMI>4). Cells were then grouped based on gRNA identity. For differential gene expression analysis, the transcriptomic profiles of cells sharing a gRNA were compare to cells with only non-targeting gRNAs using Seurat's FindMarkers function to test for differentially expressed genes (DEGs) with the hurdle model implemented in MAST. Upregulated DEGs were input into EnrichR's GO Biological Process 2021 database (Kuleshov, M. V. et al. Nucleic Acids Research 2016, 44, 90-97) for functional annotation.

RNA-sequencing with BATF3 Overexpression. CD8+ T cells were transduced with lentivirus encoding for BATF3-2A-GFP or GFP and expanded for 10 days. On day 10, 4×10⁵ GFP+ T cells were sorted for subsequent RNA isolation using Norgen's Total RNA Purification Plus Kit. RNA was submitted to Azenta (formerly Genewiz) for standard RNA-seq with polyA selection. Reads were first trimmed using Trimmomatic (Bolger, A. M., et al. Bioinformatics 2014, 30) v0.32 to remove adapters and then aligned to GRCh38 using STAR v2.4.1a aligner. Gene counts were obtained with featureCounts (Liao, Y., et al. Bioinformatics 2013, 30) from the subread package (version 1.4.6-p4) using the comprehensive gene annotation in Gencode v22. Differential expression analysis was determined with DESeq2 (Love, M. I., et al. Genome Biology 2014) where gene counts are fitted into a negative binomial generalized linear model (GLM) and a Wald test determines significant DEGs (P_{adj}<0.05). Upregulated and downregulated DEGs were input into DAVID's biological processes database (Jr, G. D. et al. Genome Biology 2003) for functional annotation.

Single cell RNA-seq analysis of CD19 CAR T cell infusion product for responders and non-responders. scRNA-seq data of the infused CD19 CAR T cell products from patients treated with tisagenlecleucel (Haradhvala, N.J. et al. Nature Medicine 2022, 28, 1848-1859) were downloaded from GEO:GSE197268. Patient data in MarketMatrix format were classified as responders (R) and non-responders (NR) and processed with Seurat (Hao, Y. et al. Cell 2021, 184, 3573-3587 e3529) 4.2.0. For each patient, cells with fewer than 20% mitochondrial UMI counts, more than 20 gene expression (GEX) UMI counts, and in the bottom 95th percentile of GEX UMI counts were selected. GEX UMI counts were log-normalized for further analysis. Individual patient data were merged (merge function in Seurat) into a combined Seurat object, preserving the group identity in the cellular barcodes. GEX UMI counts were linearly scaled and centered (ScaleData function with default parameters) before finding the most differentially expressed genes (Seurat FindVariableFeatures) using principal component analysis (PCA). Clustering was performed using the first 10 principal components to identify and select CD8+ T cells for subsequent analyses. MAST was used to identify differentially expressed genes between CD8+ T cells from responders and non-responders.

ATAC-seq. 5×10⁴ transduced CD8+ T cells were sorted for Omni ATAC-seq as previously described (Corces, M. R. et al. Nature Methods 2017, 14). Libraries were sequenced on an Illumina NextSeq 2000 with paired-end 50 bp reads. Read quality was assessed with FastQC and adapters were trimmed with Trimmomatic (Bolger, A. M., et al. Bioinformatics 2014, 30). Trimmed reads were aligned to the Hg38 reference genome using Bowtie (Langmead, B., et al. Genome Biology 2009)(v1.0.0) using parameters −v 2—best—strata −m 1. Reads mapping to the ENCODE hg38 blacklisted regions were removed using bedtools2 (Quinlan, A. R. & Hall, I. M. Bioinformatics 2010, 26) intersect (v2.25.0). Duplicate reads were excluded using Picard MarkDuplicates (v1.130; http://broadinstitute.github.io/picard/). Count per million normalized bigWig files were generated for visualization using deeptools bamCoverage (Ramirez, F., et al. Nucleic Acids Research 2014, 42)(v3.0.1). Peak calling was performed using MACS2 narrowPeak (Zhang, Y. et al. Genome Biology 2008) and filtered for P_{adj}<0.001. Peak calls were merged across samples to make a union-peak set. A count matrix containing the number of reads in peaks for each sample was generated using featureCounts (Liao, Y., et al. Bioinformatics 2013, 30)(subread v1.4.6) and used for differential analysis in DESeq2 (Love, M. I., et al. Genome Biology 2014)(v.1.36). ChIPSeeker (Yu, G., et al. Bioinformatics 2015, 31, 2382-2383) was used to annotate the genomic regions and retrieve the nearest gene around each peak.

In Vitro Tumor Killing Assay. CD8+ T cells were transduced with lentiviruses encoding for a HER2-CAR-mCherry at 24 hours post-activation and BATF3-2A-GFP or GFP at 48 hours post-activation. After 12 days of expansion, CAR+GFP+ T cells were sorted and counted for the co-culture assay. Four hours before starting the co-culture, 2×10⁵ HER2+SKBR3 s were plated in a 24 well plate with cDMEM to allow the SKBR3 s to adhere to the plate. After four hours, cDMEM was discarded and mCherry+GFP+ T cells in cPRIME media were added at the indicated effector to target (E:T) cell ratios. After 24 hours of co-culture, the cells were harvested by collecting the supernatant (containing T cells and dead tumor cells) and adherent cells (which were detached from the plate using trypsin). Cells were spun down at 600×g for 5 min and then stained with a fixable viability dye (FVD) and Annexin V to label dead and apoptotic cells according to manufacturer's protocol. Stained cells were analyzed using flow cytometry. The percentage of viable tumor cells was quantified using the following strict gating strategy. First, T cells were excluded based on cell size and GFP signal. Next, a gate was set around the double negative (FVD−, Annexin V−) fraction containing viable tumor cells and cellular debris. Visualizing these events on SSC vs. FSC, a gate was set to encompass events located in the bottom left quadrant. This gate was then inverted to exclude debris from the viability calculation and moved immediately beneath the T cell exclusion gate on the gating hierarchy. Tumor viability was reported using the percentage of tumor cells in the final double negative (FVD−, Annexin V−) gate.

CD3/CD28 and Tumor Repeat Stimulations. For repeated rounds of CD3/CD28 dynabead stimulation, CD3/CD28 beads were removed, cells were counted, replated at 1-2.5×10⁵ T cells, and restimulated with new CD3/CD28 beads at a 3:1 bead to cell ratio in a 24 well plate every 3 days. On day 12, cells were stained and analyzed for expression of exhaustion-associated markers using flow cytometry. For repeated rounds of tumor stimulation, 1×10⁵ HER2 CAR T cells were transferred to a new 24 well plate with 2×10⁵ SKBR3 s for a 1:2 E:T ratio every 3 days. T cells were recovered without antigen stimulation for two days after the final round of tumor stimulation before ATAC-seq on day 14. For both modes of chronic stimulation, T cells were restimulated on days 3, 6, and 9.

Mice. 6-8-week-old female immunodeficient NOD/SCID gamma (NSG) mice were obtained from Jackson Laboratory and then housed and handled in pathogen-free conditions.

In Vivo Tumor Model. 2.5×10⁶ HCC1954 cells were implanted orthotopically into the mammary fat pad of NSG mice in 100 μL 50:50 (v:v) PBS:Matrigel. T cells were expanded for 9-11 days post-transduction before treatment. Transduction rates were measured on the day of treatment using flow cytometry. For all in vivo experiments, transduction rates exceeded 80% for both HER2-CAR-2A-GFP and HER2-CAR-2A-BATF3 constructs. T cells were resuspended at 50×10⁶ CAR+ cells mL⁻¹ in 1×PBS and serially diluted to the appropriate cell concentrations for 200 μL injections of either 10×10⁶, 2×10⁶, 5×10⁵, 2.5×10⁵, or 1×10⁵ HER2 CAR+ T cells. 21 days after tumor implantation, and immediately prior to CAR T cell injections, mice were randomized into groups and tumors measured. Tumor volumes were calculated based on caliper measurements using the formula volume: =% (Length×Width²). CAR T cells were injected intravenously by tail vein injection. Tumors were measured every 4-6 days.

Statistics. Statistical details for all experiments can be found in the figure legends. ns=not significant, *<0.05, **<0.01, ***<0.001, ****<0.0001.

### Example 2

### CRISPRi/a Screen Reveal Canonical and Novel Transcriptional Regulators of T Cell Fate

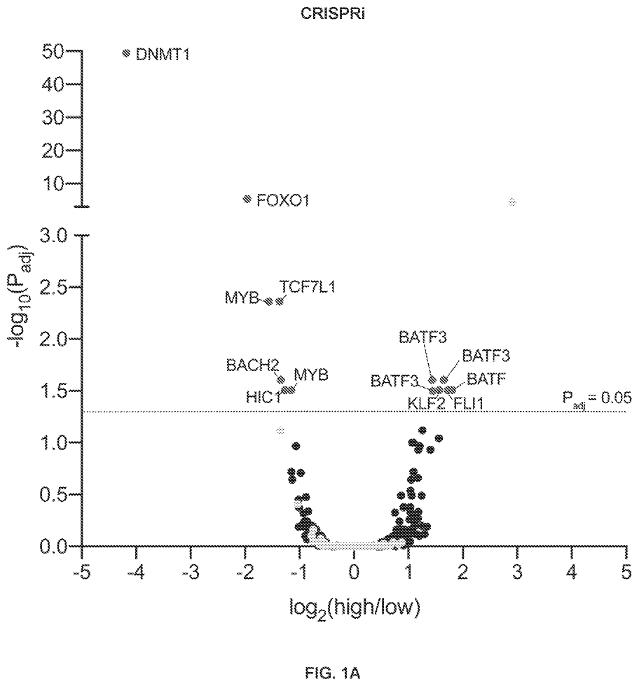

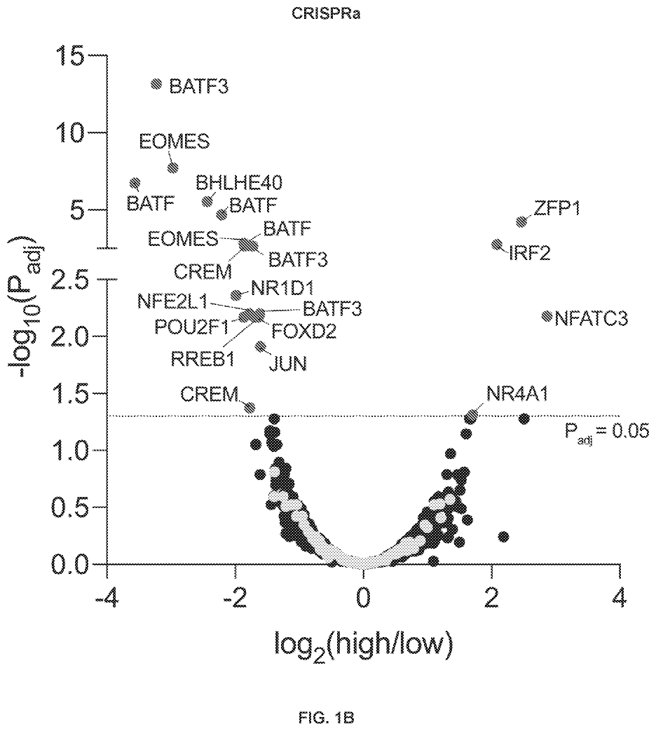

Several positive transcriptional regulators of memory T cells were identified including FOXO1, MYB, TCF7L1, and BACH2 in the CRISPRi screen (FIG. 1A) with respective gRNAs all enriched in the CCR7 low bin, consistent with the expectation that downregulation of memory-associated TFs should reduce CCR7 expression. Conversely, activation of EOMES and JUN led to decreased CCR7, which is consistent with their well-characterized roles as a molecular drivers of T cell differentiation and effector response. Interestingly, a DNMT1-targeting gRNA had the largest effect size in the CRISPRi screen. DNMT1 encodes for DNA methyltransferase I, an epigenetic-modifying protein that actively maintains DNA methylation across cell divisions by recognizing and binding to hemimethylated DNA. The direction of DNMT1's effect is consistent with clinical trial data showing that TET2 (a DNA demethylase) knockout increased the memory T cell population. Unexpectedly, multiple gRNAs targeting both BATF and BATF3 were enriched in the CCR7 high and low bins for the CRISPRi and CRISPRa (FIG. 1B) screens, respectively. BATF overexpression has been shown to counter T cell exhaustion in human T cells, while BATF3 is critical for CD8+ T cell memory formation and rapid recall response in mouse T cells. The role of BATF3 in human T cells, however, has largely been unexplored. Both repression and activation screens also revealed less defined factors: HIC1, FLI1, KLF2, BHLHE40, CREM, NR1D1, POU2F1, NFE2L1, FOXD2, RREB1, ZFP1, IRF2, NFATC3, and NR4A1 for T cell reprogramming.

### Example 3

### CRISPRi Mediated Silencing of DNMT1 and FOXO1 Reduces Expression of Other Memory-Associated Makers

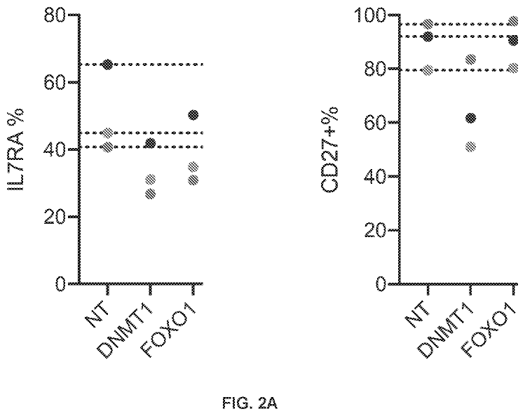

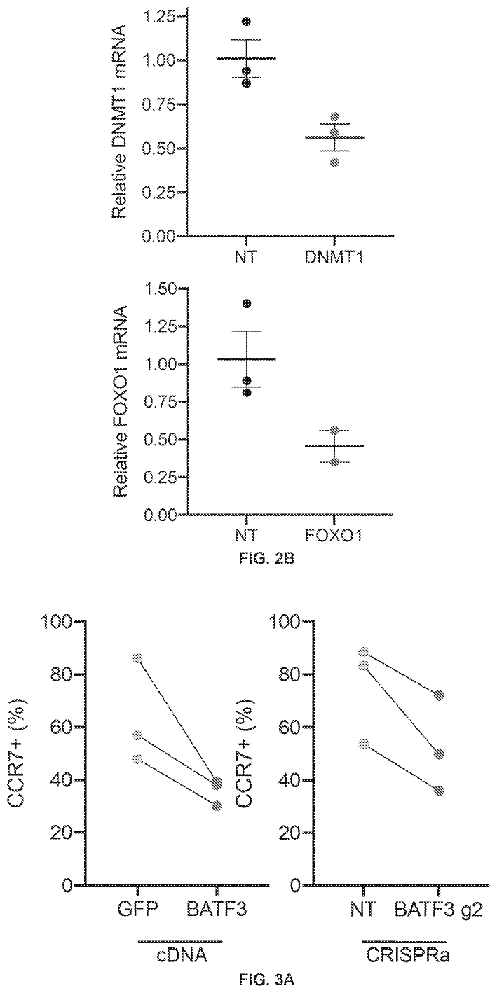

Although our CRISPRi/a screens relied on a single reporter gene CCR7, changes to CCR7 expression was examined to see if it would serve as a proxy for cellular reprogramming with widespread gene expression changes. The effects of DNMT1 and FOXO1 silencing on other memory-associated markers were tested, and it was found that DNMT1 silencing attenuated both IL7RA and CD27 expression, while FOXO1 only reduced IL7RA expression (FIG. 2A). It was confirmed that DNMT1 and FOXO1 were indeed silenced using RT-qPCR for target genes compared to cells treated with a non-targeting gRNA (FIG. 2B).

### Example 4

### CRISPRa Mediated Activation of BATF3 and BATF3 ORF Overexpression Reduces CCR7 Expression

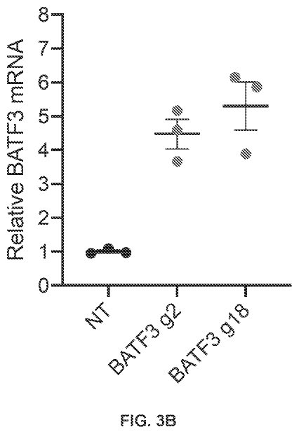

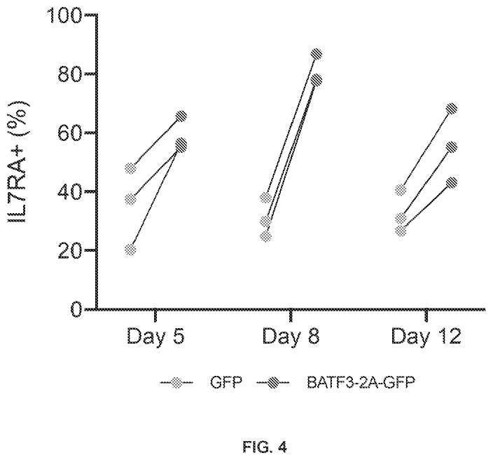

In line with our CRISPRa screen, it was found that multiple BATF3 gRNAs indeed reduced CCR7 expression levels when paired with ^{VP64}dSaCas9^{VP64}. To further support that finding, BATF3's ORF (390 bp) was cloned into a constitutive lentiviral backbone driven by the human ubiquitin C promoter (hUbc), and this construct was delivered to primary human T cells. FIG. 3A shows immunophenotyping of CCR7 on T cells treated with GFP or BATF3-2A-GFP encoding lentivirus (left panel) and non-targeting or BATF3-targeting gRNA (right panel). FIG. 3B is a graph showing gene expression for BATF3. It was found that BATF3 overexpression had a similar effect on CCR7 expression levels. Interestingly, BATF3 overexpression dramatically increased the fraction of cells expressing IL7RA throughout culture (FIG. 4). IL7RA is a known marker of memory T cells and associated with long-term T cell survival and persistence.

### Example 5

### BATF3 Attenuates T Cell Exhaustion Programs and Co-Silences Expression of Clinically Relevant External and Internal Checkpoint Inhibitors

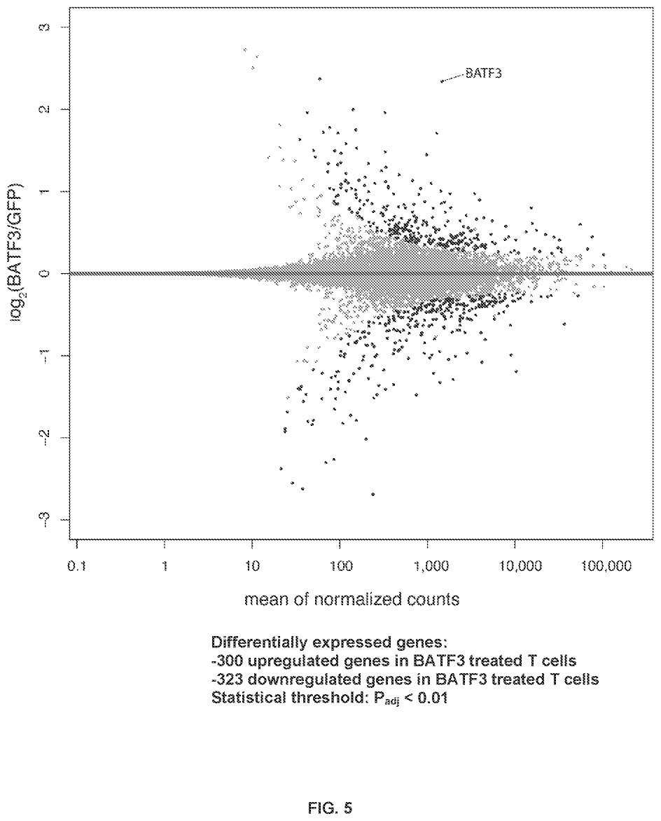

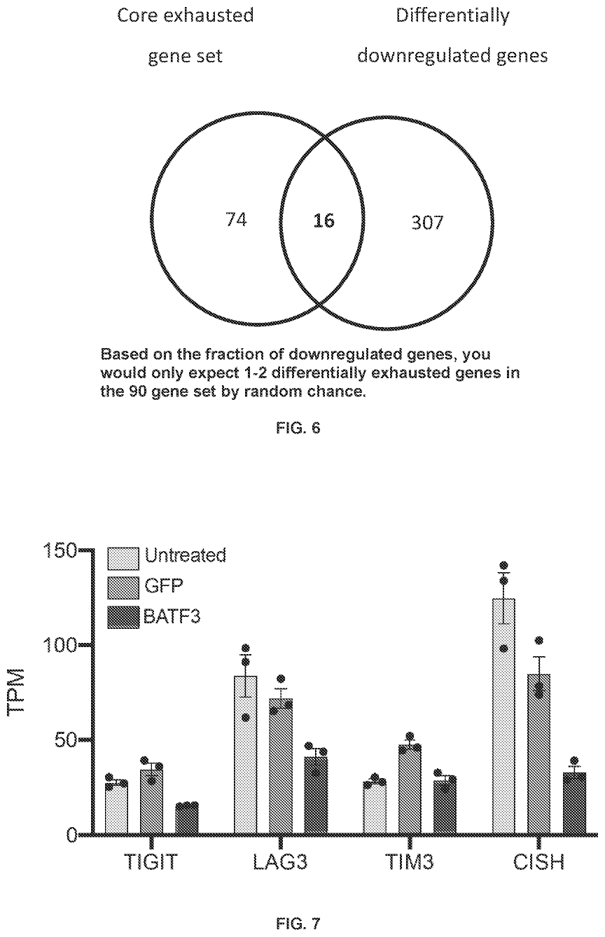

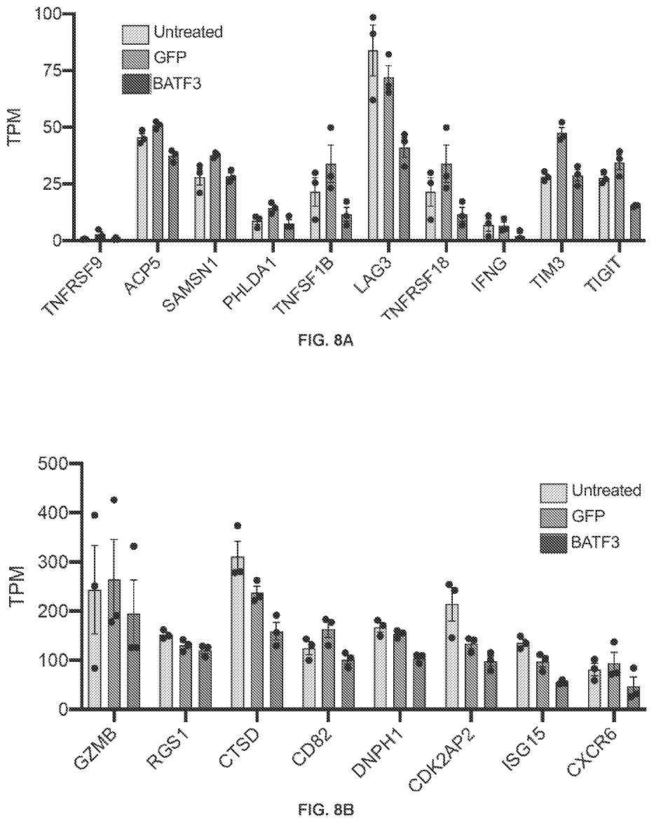

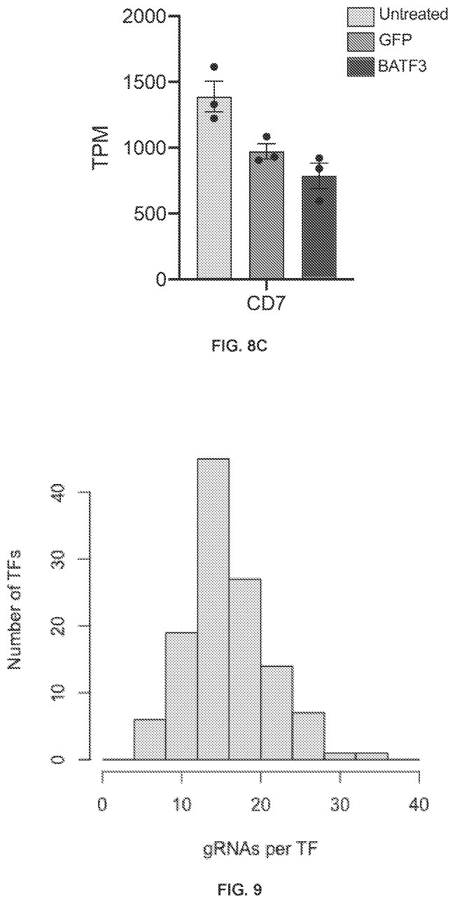

To evaluate the global effects of BATF3 overexpression, GFP or BATF3-2A-GFP lentivirus was delivered to three CD8⁺ donors, the cells were cultured for 10 days, and then RNA-sequencing (RNA-seq) was performed. FIG. 5 is a MA plot with log 2fold shrinkage after DESeq2 analysis comparing gene counts between BATF3-treated cells and GFP-treated cells, and showing that BATF3 induced widespread changes in gene expression. Differential gene expression analysis between GFP and BATF3 treated cells revealed 623 differentially expressed genes (P_{adj}<0.01) with 323 downregulated and 300 upregulated genes in BATF3-overexpressing cells. Of the 323 downregulated genes, many of them were exhaustion or effector-associated genes (FIG. 6). Interestingly, BATF3 overexpression led to co-silencing of multiple checkpoint inhibitors including TIGIT, LAG-3, TIM-3, and CISH (FIG. 7, FIGS. 8A-8C). TIGIT, LAG-3, and TIM-3 are the next wave of inhibitory receptor targets being explored in the clinic for cancer immunotherapy with clinical trials using targeted antibodies. Conversely, CISH is an internal checkpoint that regulates T cell receptor (TCR) signaling. Most of the 300 upregulated genes were related to cell cycle, DNA replication, and metabolism as BATF3 overexpression is known to increase proliferation and mitochondrial fitness. BATF3 overexpression or endogenous activation of BATF3 has tremendous potential in T-cell based therapies as BATF3 can simultaneously dampen multiple arms of T cell exhaustion, increase T cell proliferation, and improve mitochondrial fitness.

### Example 6

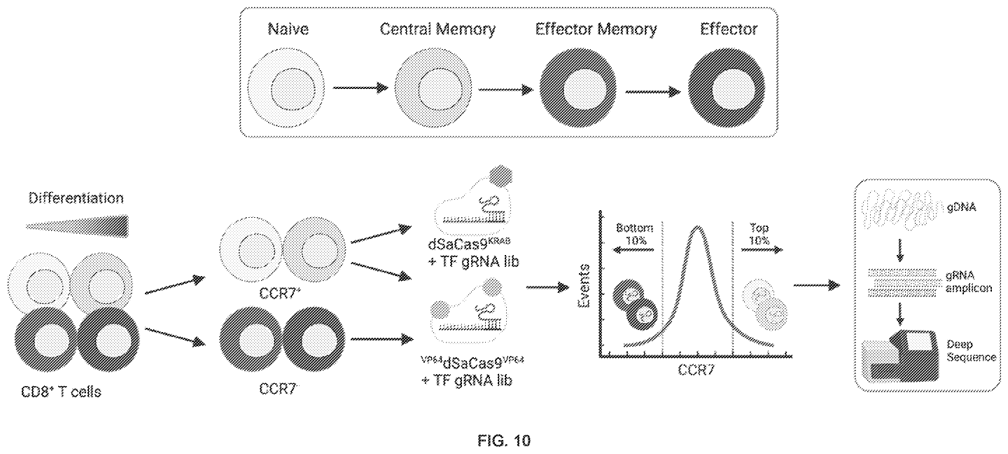

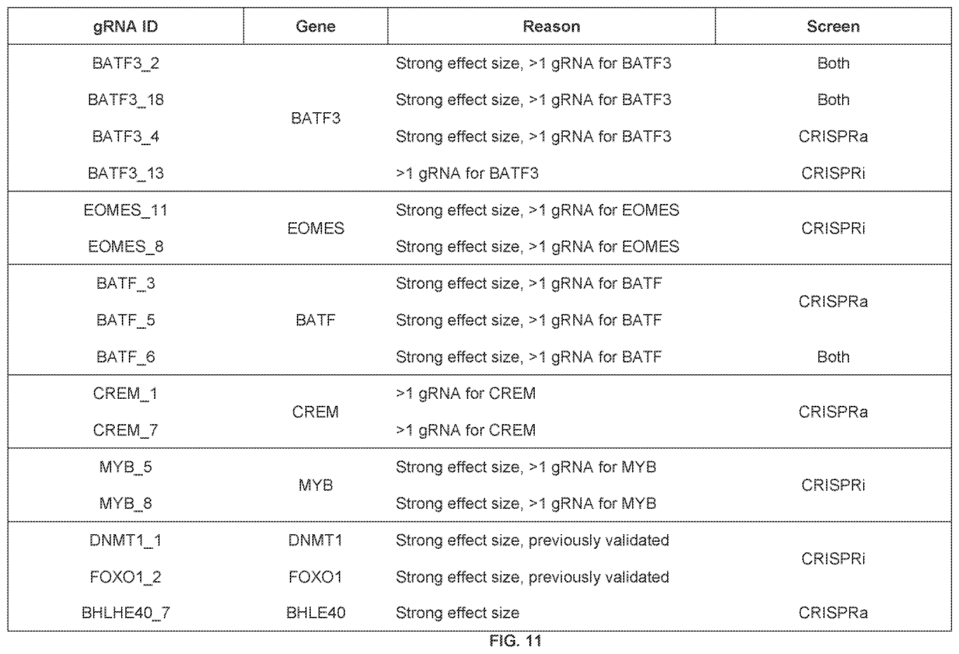

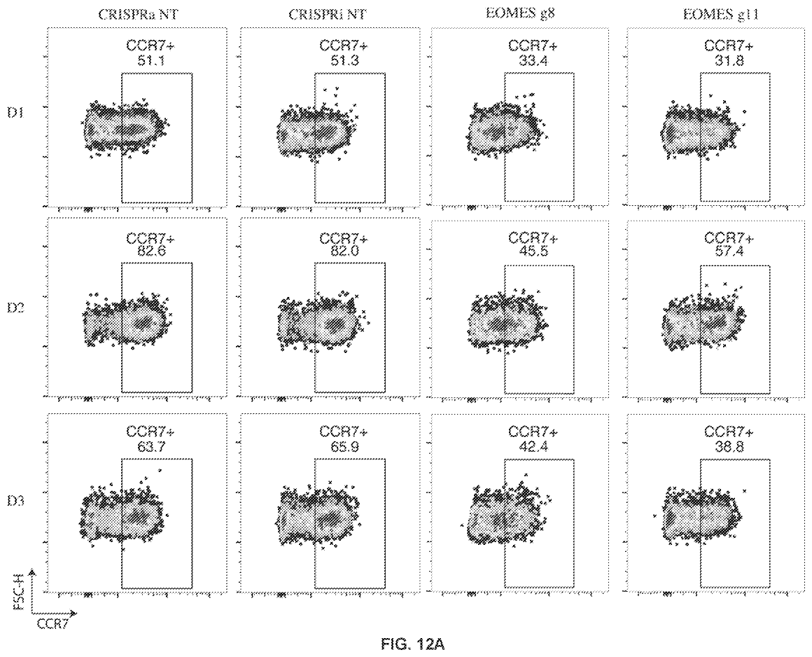

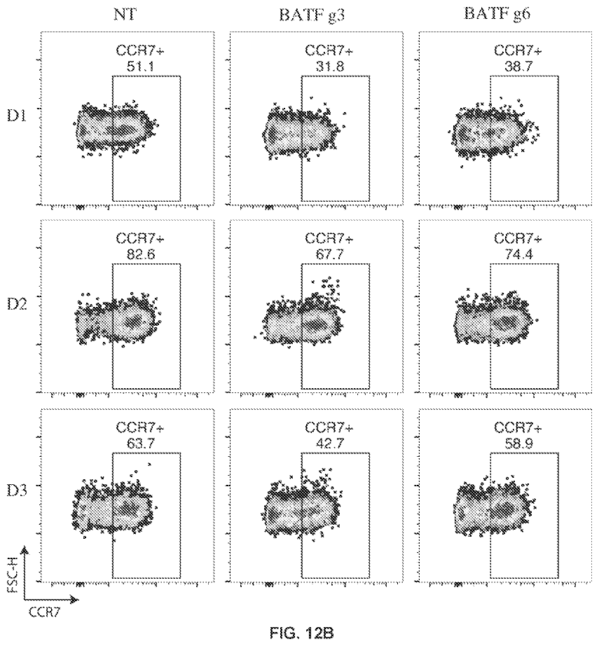

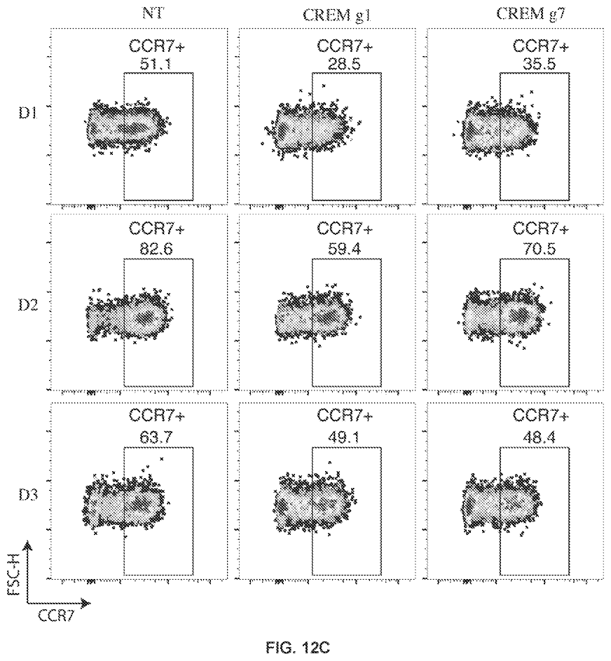

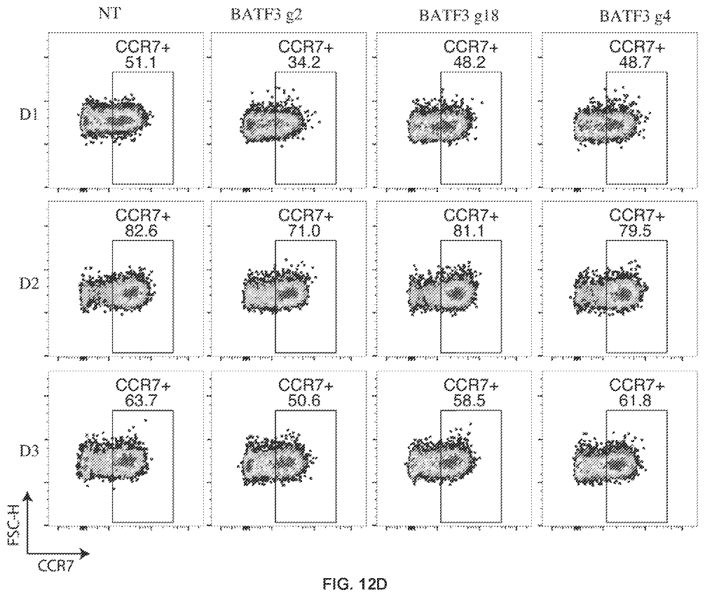

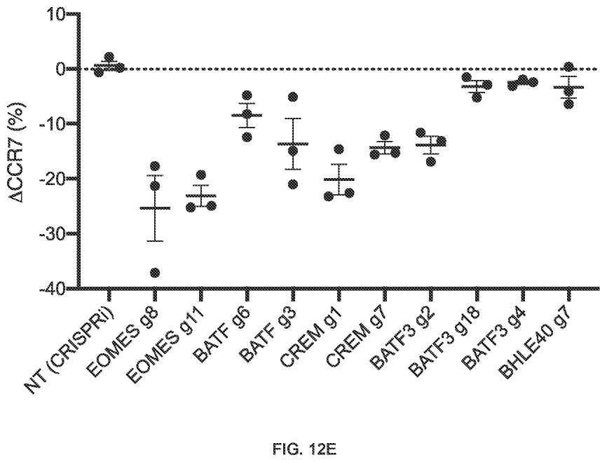

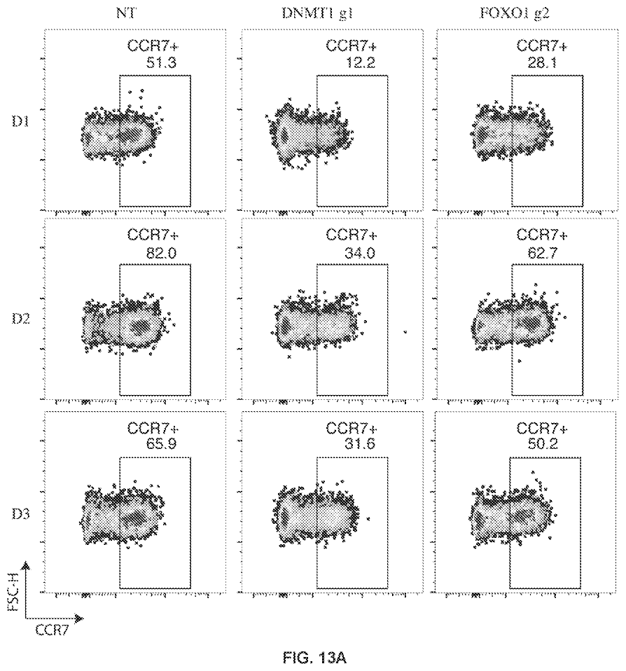

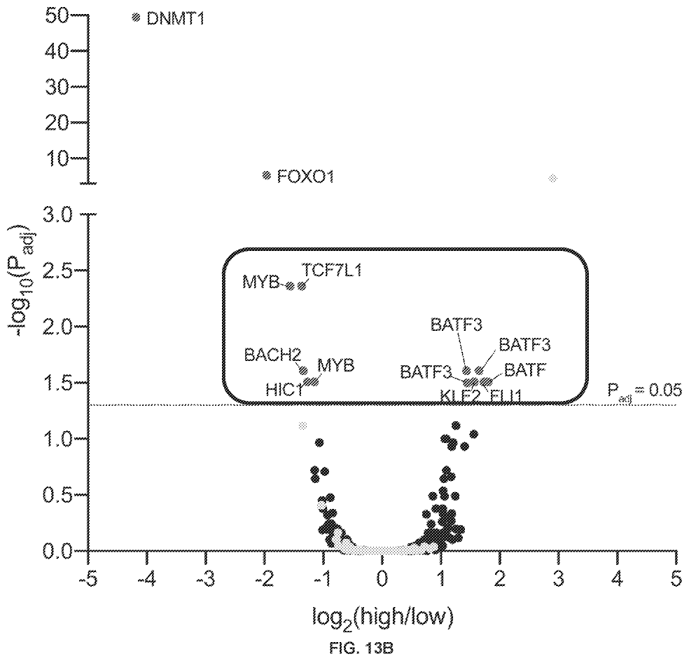

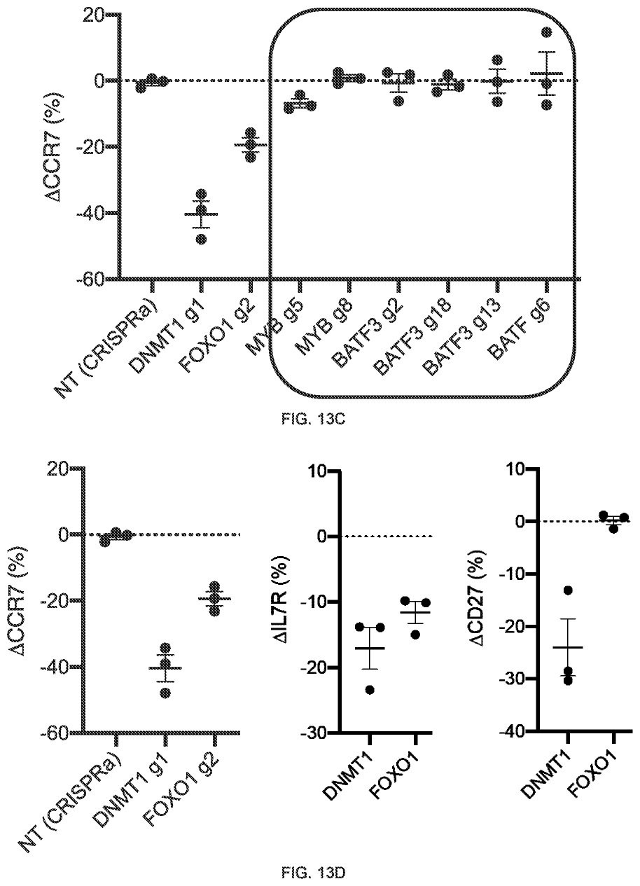

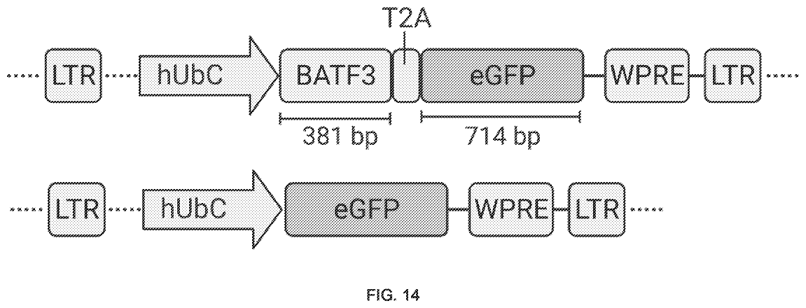

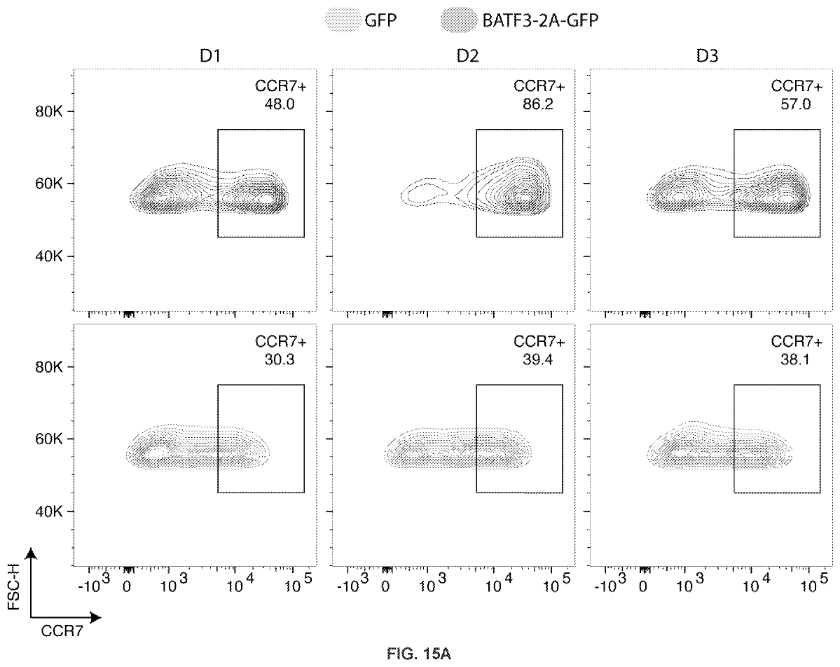

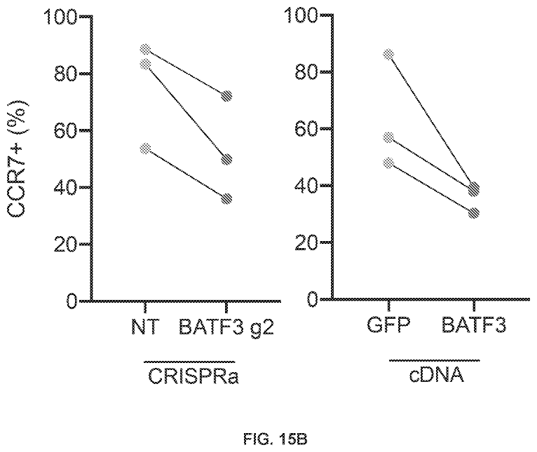

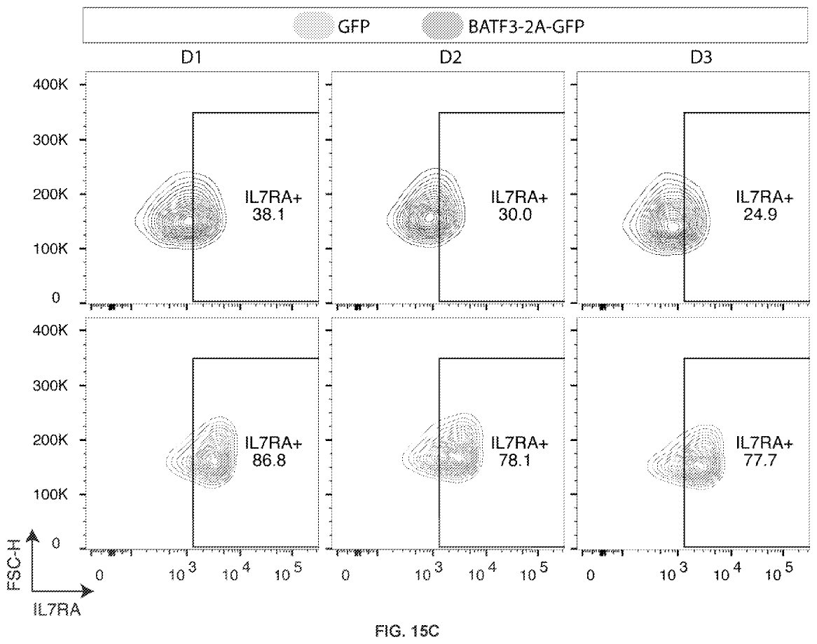

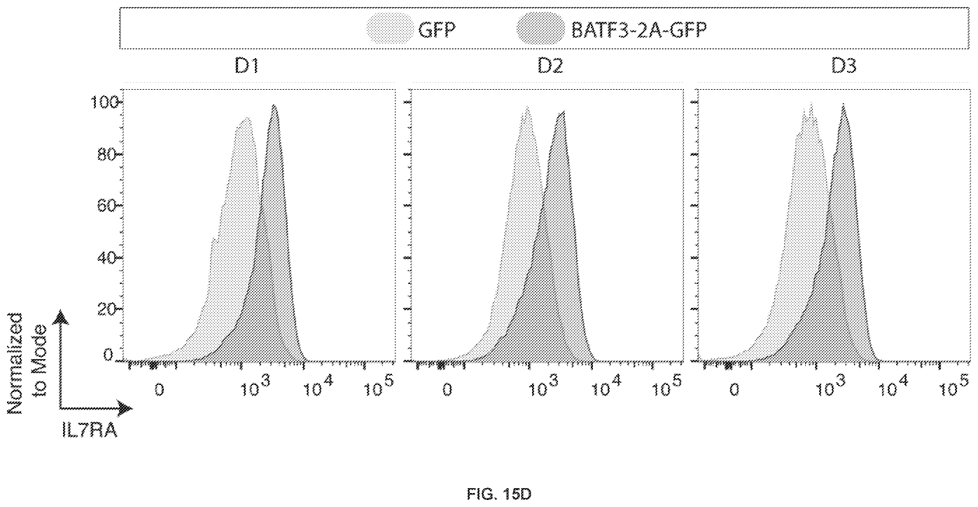

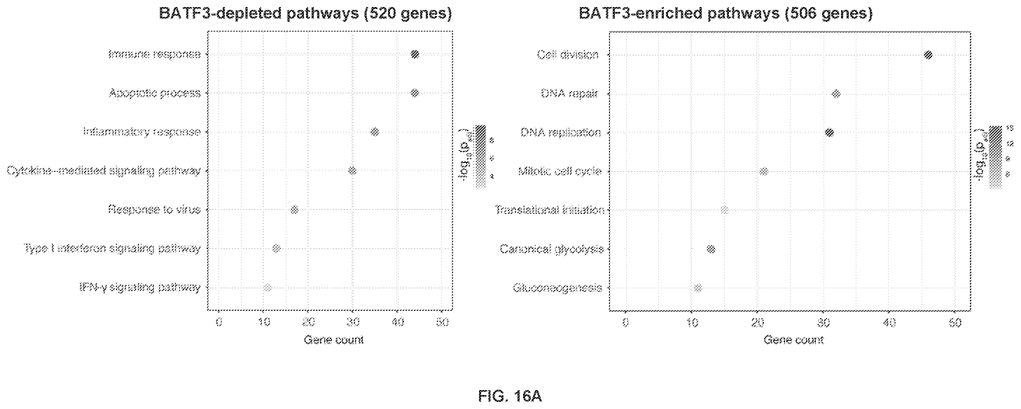

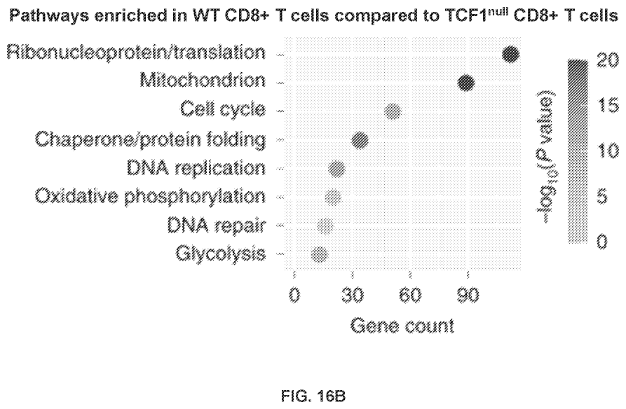

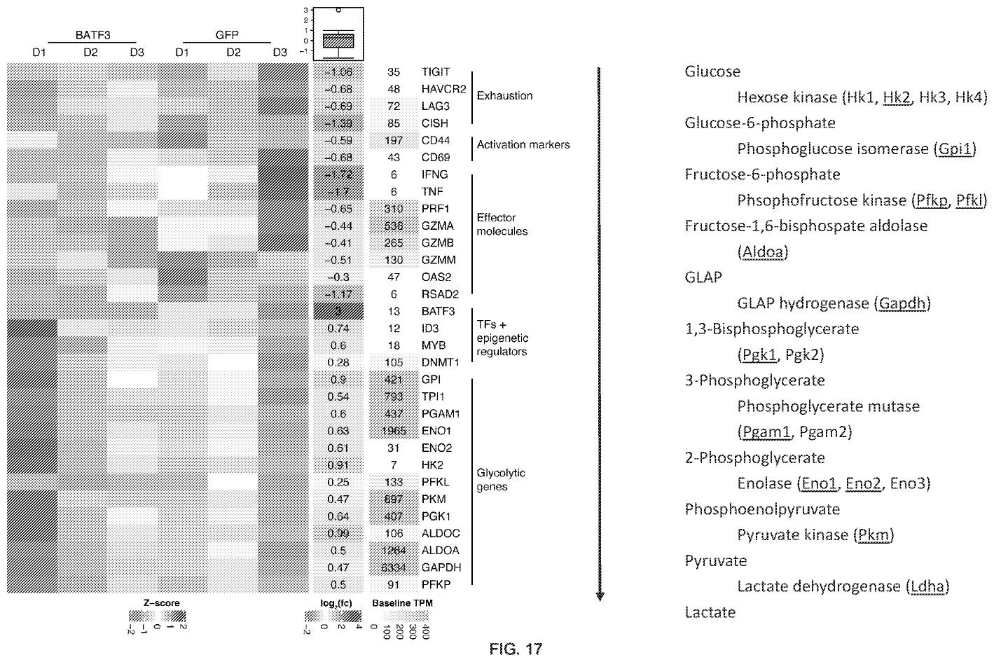

Further analyses of the CRISPRi and CRISPRa screens were completed, with results shown in FIGS. 9, 10, 11, 12A, 12B, 12C, 12D, 12E, 13A, 13B, 13C, 13D, 14, 15A, 15B, 15C, 15D, 16A, 16B, and 17.

### Example 7

### Development and Characterization of Compact and Efficient dSaCas9-Based Epigenome Editors for Targeted Gene Regulation in Primary Human T Cells

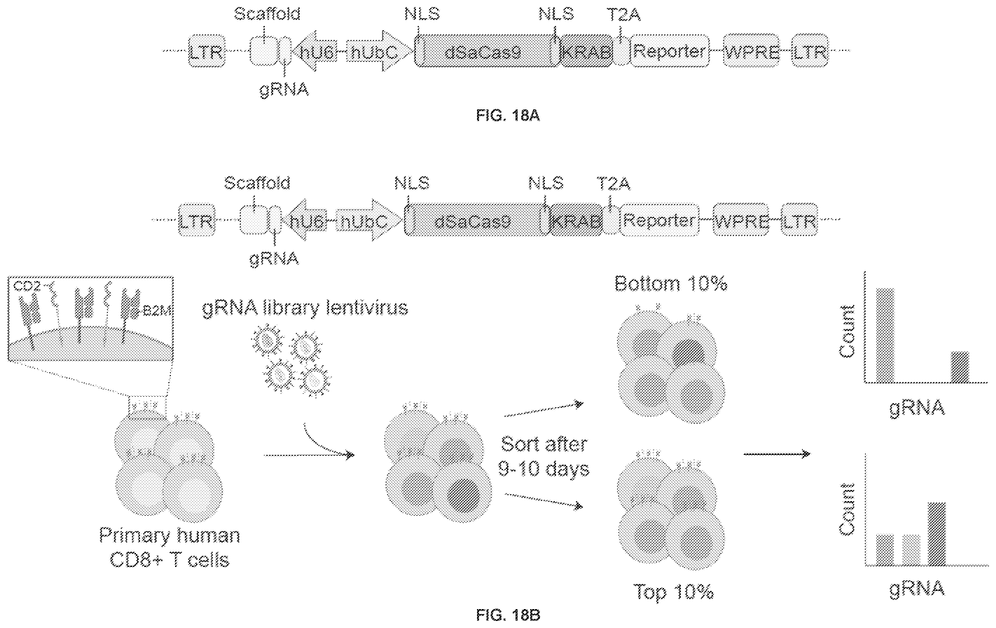

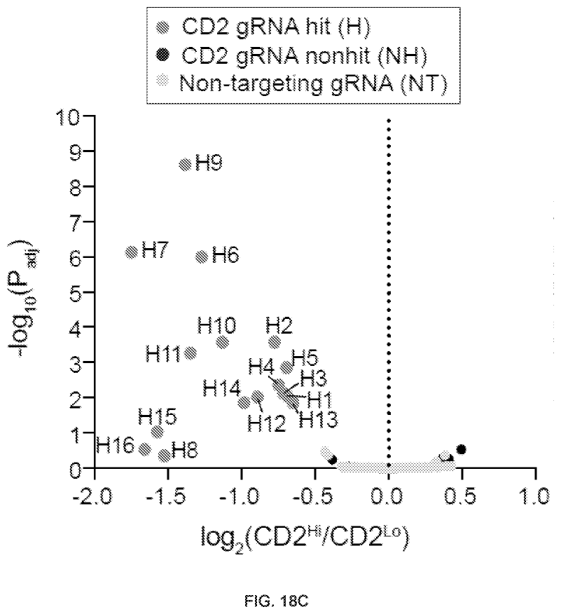

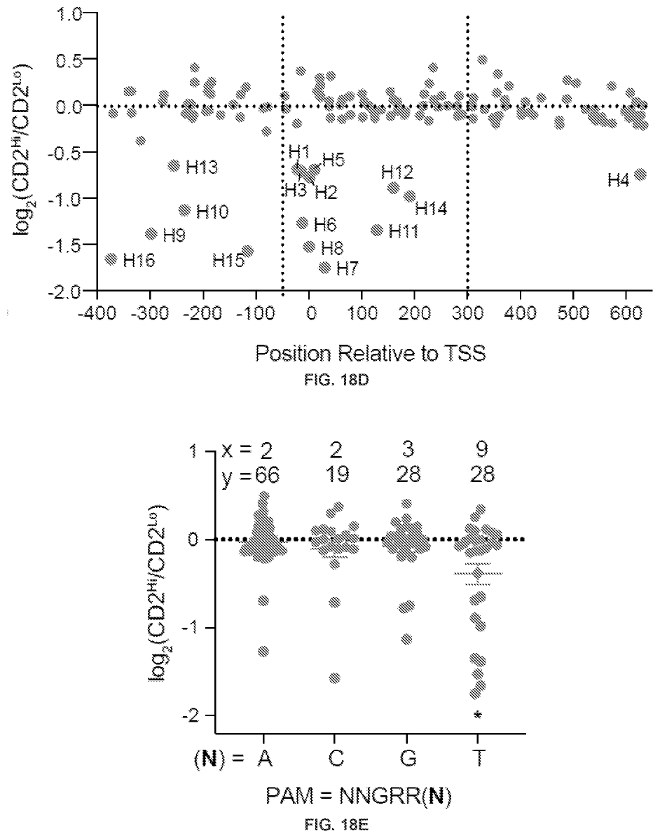

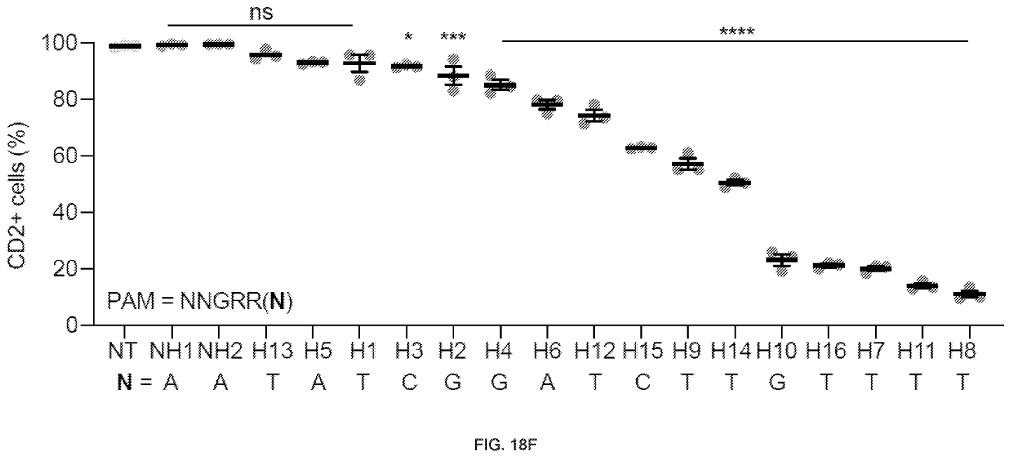

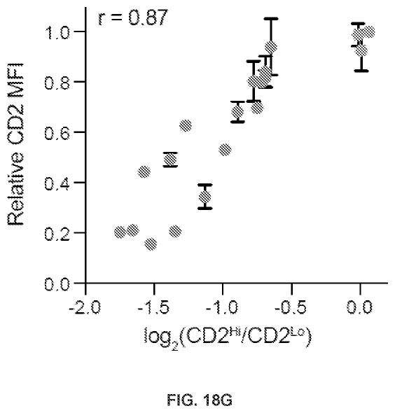

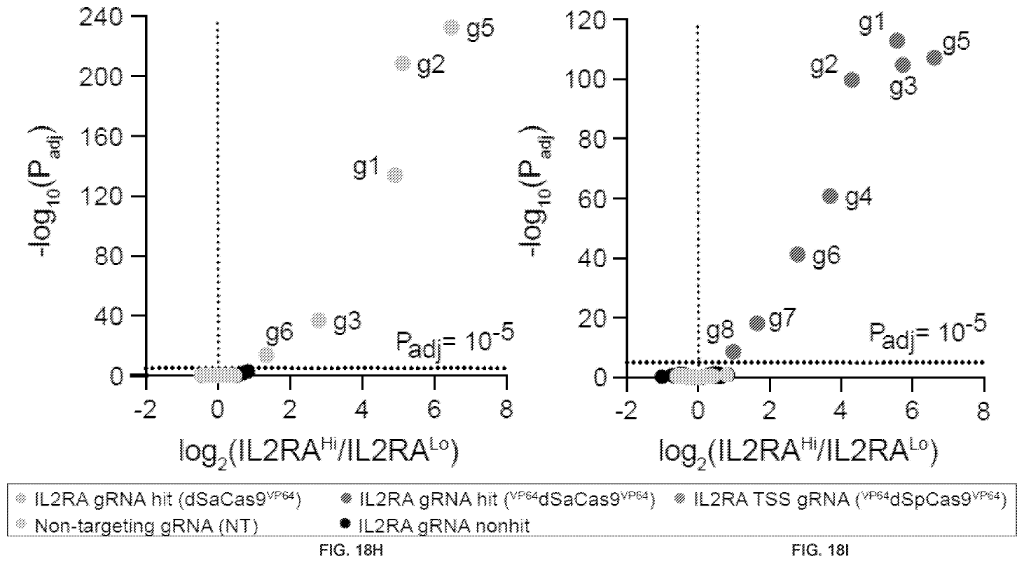

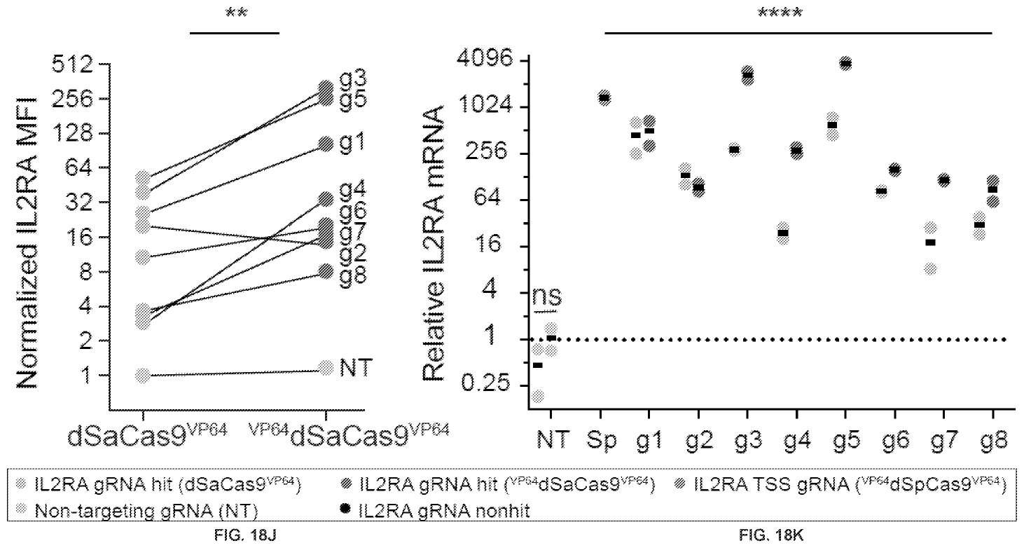

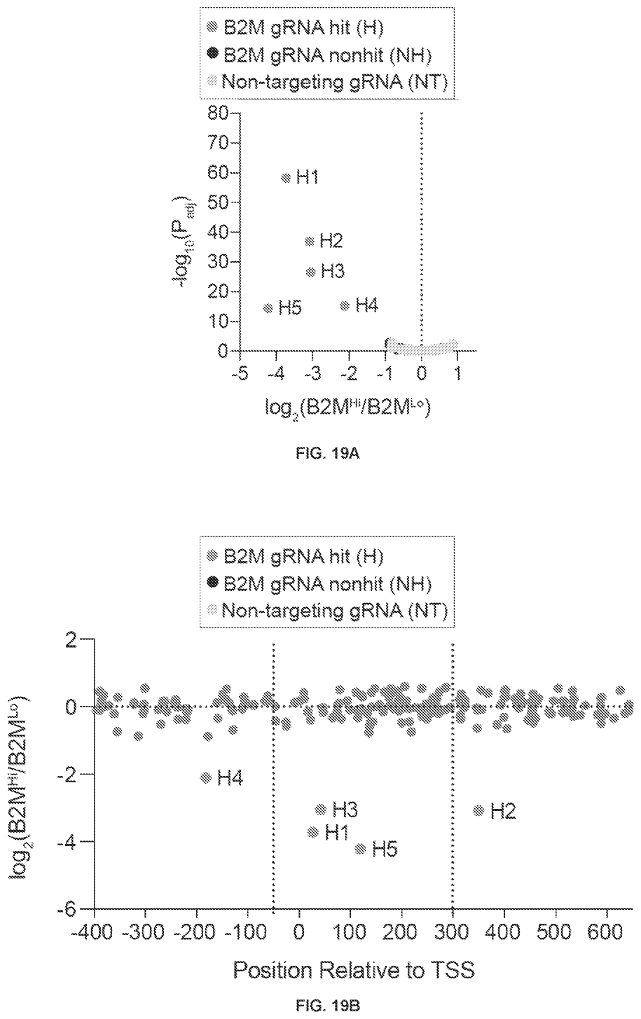

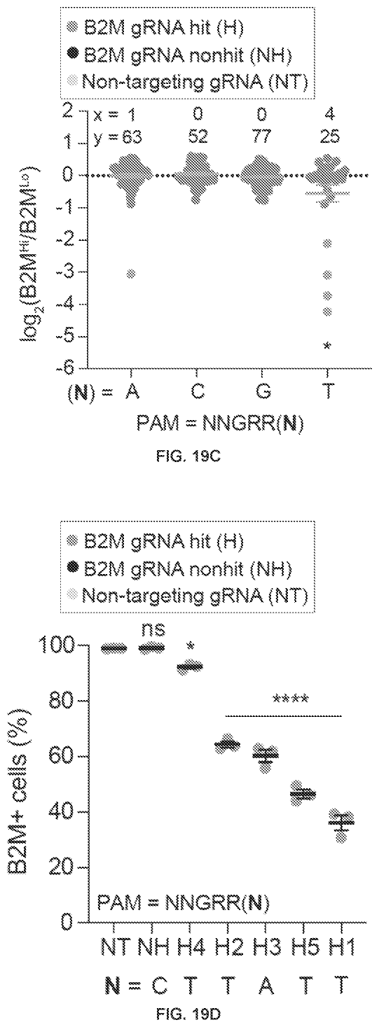

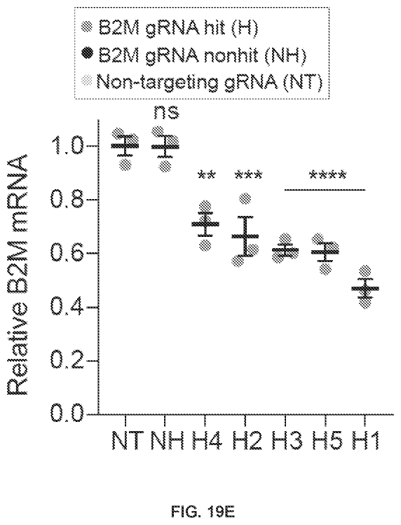

SaCas9 has been extensively used for genome editing in vivo as its compact size (3,159 bp) enables packaging into adeno-associated virus (AAV). However, SaCas9 has been used sparingly as an epigenome editor for targeted gene regulation and has not been used in the context of an epigenome editing screen. Using the smaller catalytically dead SaCas9 (dSaCas9) could circumvent the challenges that some have observed as an obstacle in using the larger S. pyogenes Cas9 (SpCas9) expressed from lentiviral vectors in primary human T cells. First, we validated that dSaCas9 fused to the KRAB repressor domain could efficiently silence gene expression in primary human T cells. We developed an all-in-one CRISPRi lentiviral vector encoding dSaCas9 fused to KRAB and a gRNA cassette driven by a human U6 promoter (FIG. 18A). Using the less restrictive protospacer adjacent motif (PAM: 5′-NNGRRN-3′) for SaCas9, we then designed and cloned independent gRNA libraries tiling ˜1,000 bp windows around the promoters of CD2 and B2M, both of which are ubiquitously and highly expressed genes encoding for surface markers to facilitate cell sorting-based screens. The CD2 and B2M gRNA libraries contained 141 and 217 targeting gRNAs in addition to 250 non-targeting gRNAs. We transduced primary human T cells with the respective gRNA library and expanded the cells for 9-10 days before staining and sorting transduced cells in the lower and upper 10% tails of CD2 or B2M expression (FIG. 18B). We recovered 16 and 5 targeting gRNAs enriched in the low bins for the CD2 and B2M CRISPRi screens without any enrichment of non-targeting gRNAs or gRNAs in the high bin (FIG. 18C and FIG. 19A). Many significantly enriched gRNAs targeted DNA regions within the previously defined optimal window (Sanson, K. R. et al. Nature Communications 2018, 9, 5416)(−50 to +300 bp of the transcriptional start site (TSS)) for gene silencing (FIG. 18D and FIG. 19B). Consistent with PAM characterization of SaCas9 for nuclease activity (Ran, F. A. et al. Nature 2015, 520, 186-191), we observed a strong gRNA preference for NNGRRT compared to NNGRRV PAMs for gene silencing (FIG. 18E and FIG. 19C).

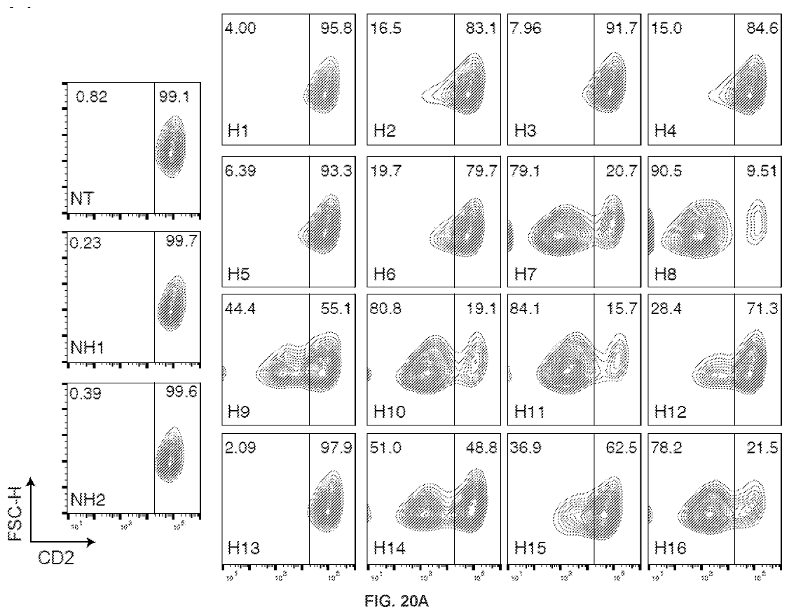

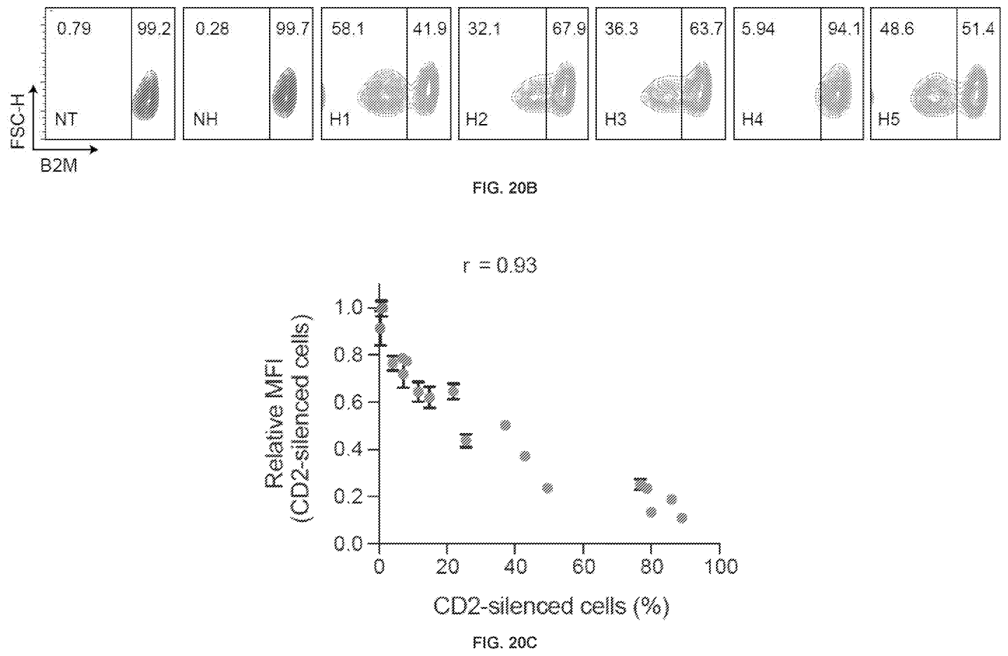

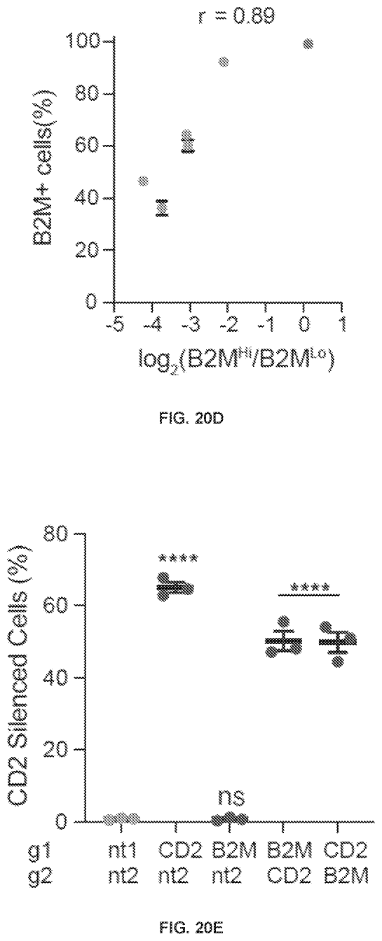

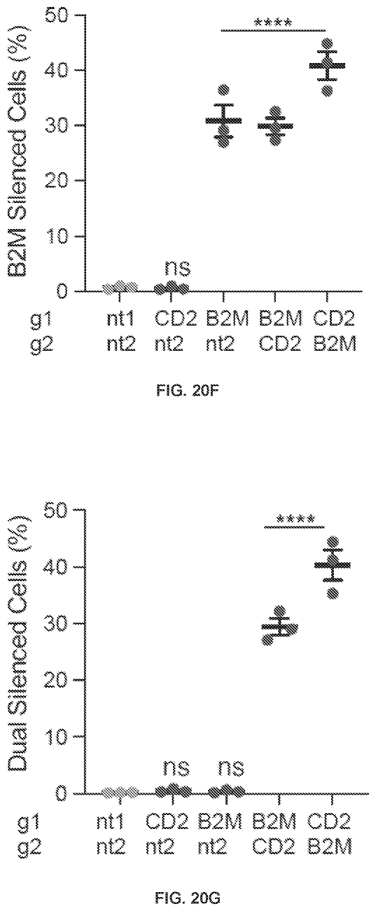

Subsequent validation of CD2 and B2M gRNA screen hits revealed marked gene silencing and a wide range of activity across gRNAs, underscoring the unique capability of CRISPRi to tune, rather than ablate, gene expression levels (FIG. 18F, FIGS. 19D-19E, and FIGS. 20A-20B). For example, the percentage of CD2 silenced cells varied from 7% to 89% depending on the gRNA (FIG. 18F and FIG. 20A). The mean expression of CD2 in silenced cells was highly correlated with the percentage of silenced cells, indicating that the effect of a gRNA across a cell population is coupled to the magnitude of gRNA activity at a single cell level (FIG. 20C). The strength of gene silencing for each gRNA was associated with its corresponding PAM with the most potent CD2 and B2M gRNAs targeting genomic sites adjacent to NNGRRT PAMs (FIG. 18F and FIG. 19D). There was also strong correlation between individual gRNA activity in validation experiments and fold-enrichment of the gRNA in the screen (FIG. 18G and FIG. 20D). Importantly, we did not observe any changes in target gene expression levels in cells treated with targeting gRNAs that were not hits in our screens, confirming our screens were both highly sensitive and specific (FIG. 18F and FIG. 19D).

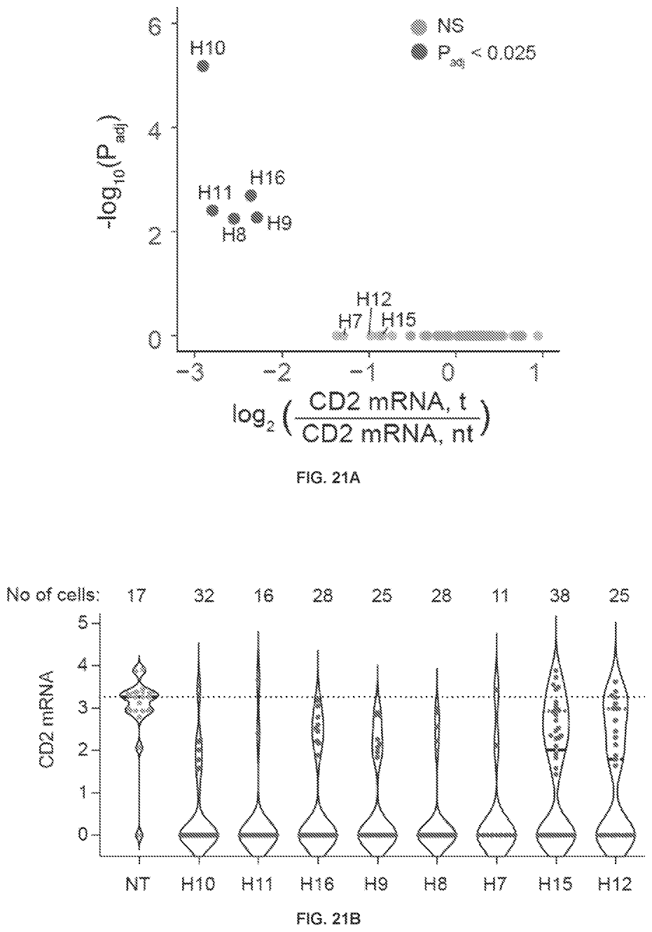

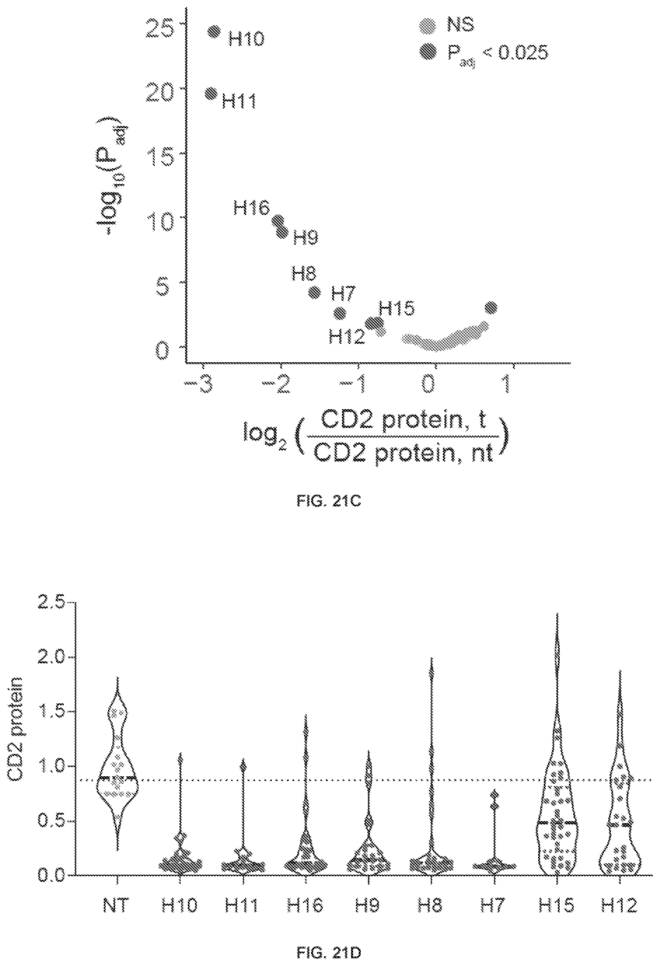

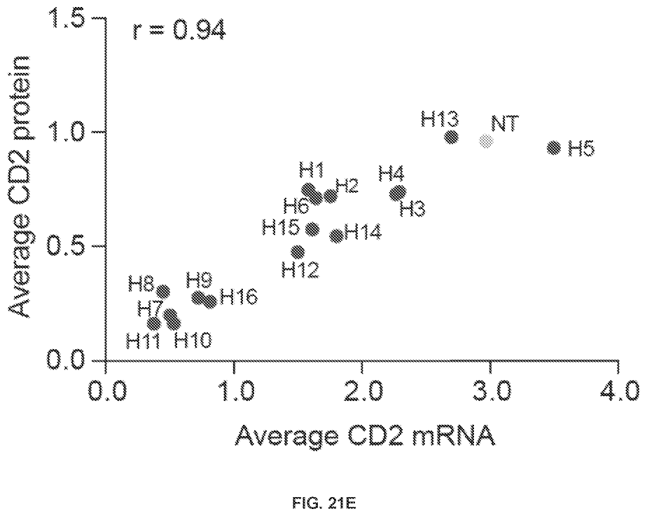

To further illustrate the robustness and versatility of this CRISPRi system, we demonstrated multiplex gene silencing and compatibility with multi-omic single cell RNA sequencing (scRNA-seq) technologies. First, we modified our CRISPRi system to enable multiplex gene silencing using orthogonal mouse and human U6 promoters in the same lentiviral vector. We tested this system using the most potent CD2 and B2M gRNAs and only detected dual silenced cells when both CD2 and B2M gRNAs were delivered (FIGS. 20E-20G). Second, we used our CD2 CRISPRi gRNA library to perform a multimodal scRNA-seq screen that recovered gRNA, mRNA, and cell hashing and CD2 protein information from single cells using 10× Genomics' 5′ standard chemistry. Adapting a protocol for 5′ capture of SpCas9 gRNAs (Mimitou, E. P. et al. Nat. Methods 2019, 16, 409-412), we spiked in a custom SaCas9 gRNA reverse transcription primer to capture non-polyadenylated gRNA transcripts for sequencing. Differential expression analyses at the mRNA and protein level revealed five and eight previously validated potent CD2 gRNA hits, consistent with reports demonstrating that analyses at the protein level have more statistical power (FIGS. 21A-21D). Moreover, CD2 mRNA and protein levels were strongly correlated across all CD2 gRNA hits (FIG. 21E).

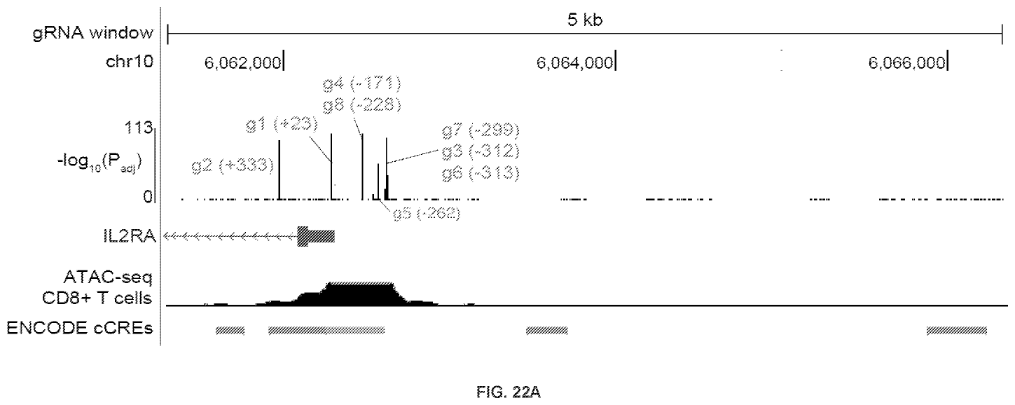

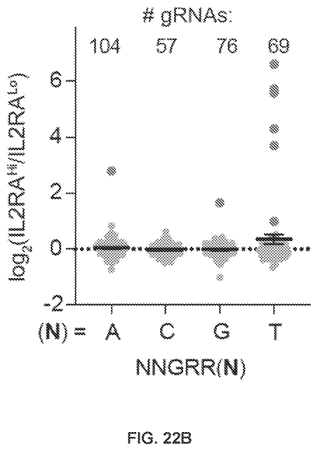

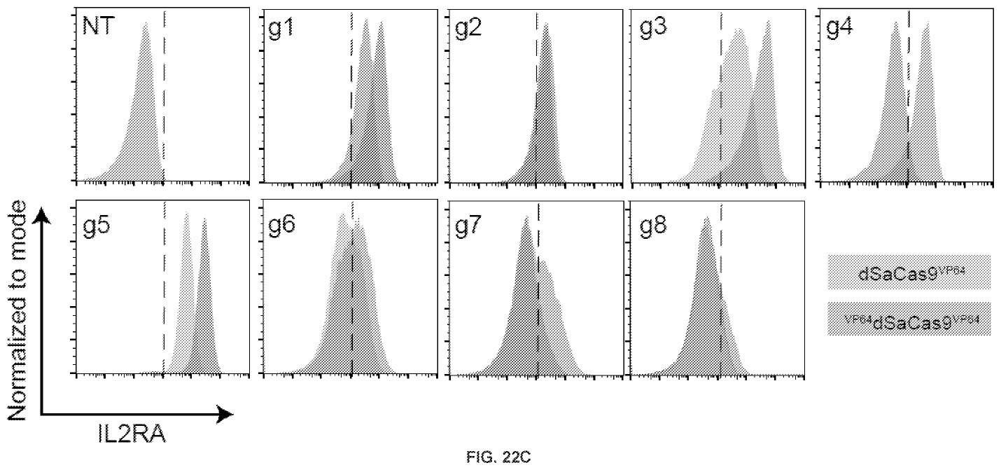

Next, we developed efficient and compact dSaCas9-based activators using the small transactivation domain VP64. Using polyclonal Jurkat cell lines constitutively expressing dSaCas9 fused to either one copy of VP64 (dSaCas9-VP64) or two copies of VP64 (VP64-dSaCas9-VP64), we conducted parallel CRISPRa screens with a gRNA library tiling a 5 kb window around the TSS of the transcriptionally silenced IL2RA gene. Interestingly, there were three more gRNA hits in the VP64-dSaCas9-VP64 CRISPRa screen along with a shared set of five gRNA hits (FIGS. 18H-181). All gRNA hits targeted sites within a prominent open chromatin peak and 350 bp of the TSS with the majority located upstream of the TSS (FIG. 22A). As with gene silencing, there was a marked preference for NNGRRT PAMs for gene activation with 75% of gRNA hits using this PAM variant (FIG. 22B).

Individual validation of all eight gRNAs in both cell lines revealed a significant increase in IL2RA expression in VP64-dSaCas9-VP64 expressing cells compared to dSaCas9-VP64 expressing cells across gRNAs (FIGS. 18J-18K and FIG. 22C). Moreover, the most potent VP64-dSaCas9-VP64gRNAs achieved equivalent levels of IL2RA gene activation as VP64-dSaCas9-VP64 paired with the best IL2RA gRNA from a published CRISPRa screen tiling the IL2RA locus in Jurkats (FIG. 18K). Given the robust performance of VP64-dSaCas9-VP64 in Jurkat cells, we constructed an all-in-one CRISPRa lentiviral vector encoding for VP64-dSaCas9-VP64 and gRNA cassette and verified its activity in primary human T cells (FIGS. 22D-22E). In summary, we developed and rigorously characterized compact and efficient dSaCas9-based epigenome editors for targeted gene regulation and high-throughput epigenetic screens in primary human T cells.

### Example 8

### CRISPR Interference and Activation Screens Map Transcriptional and Epigenetic Regulators of Human CD8 T Cell State

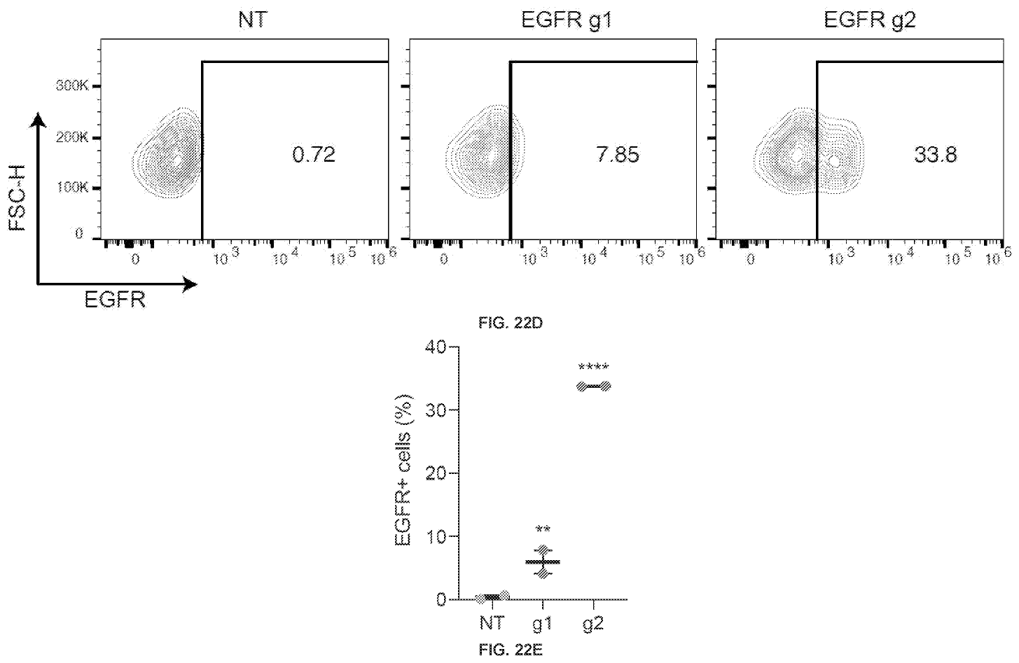

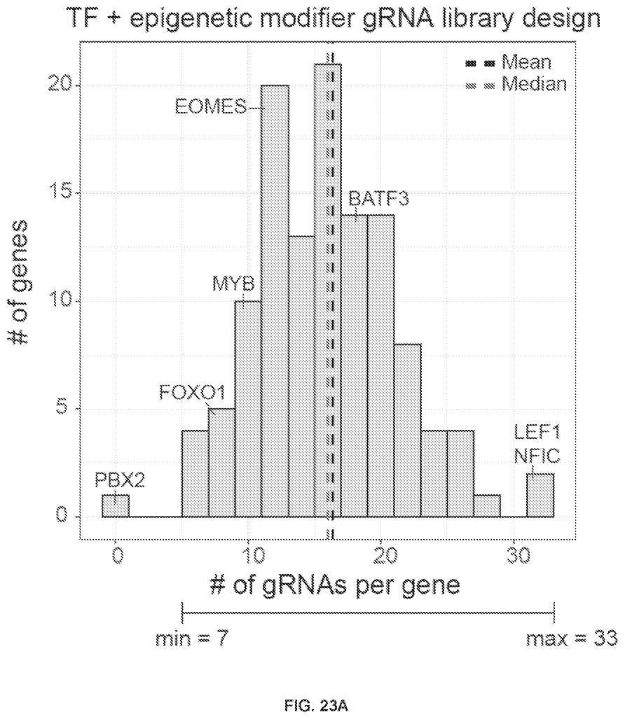

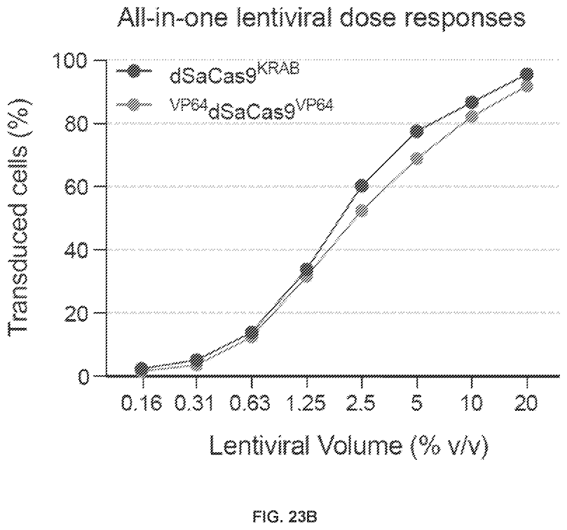

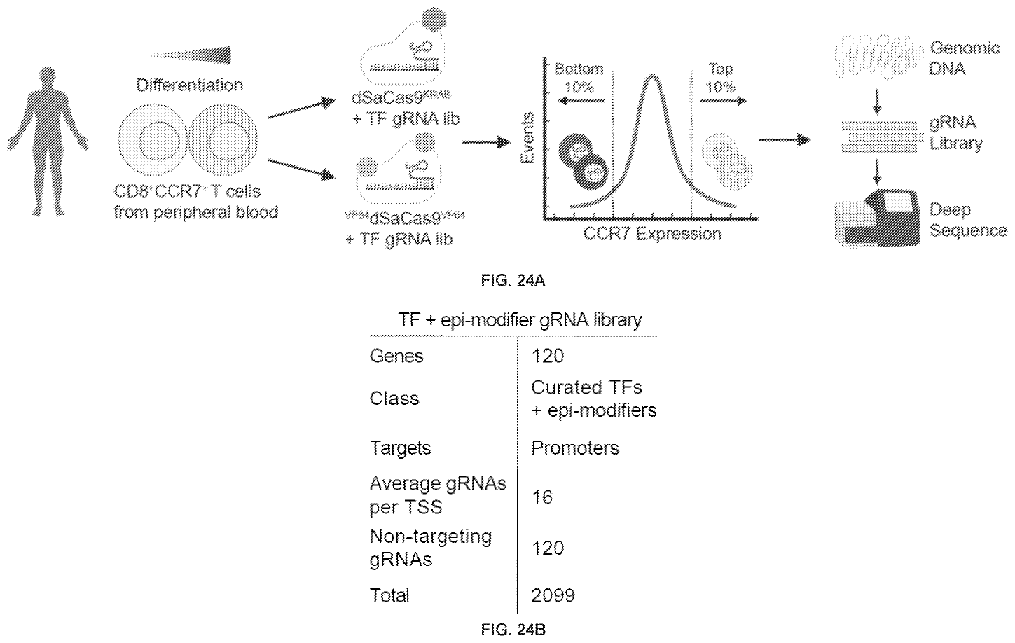

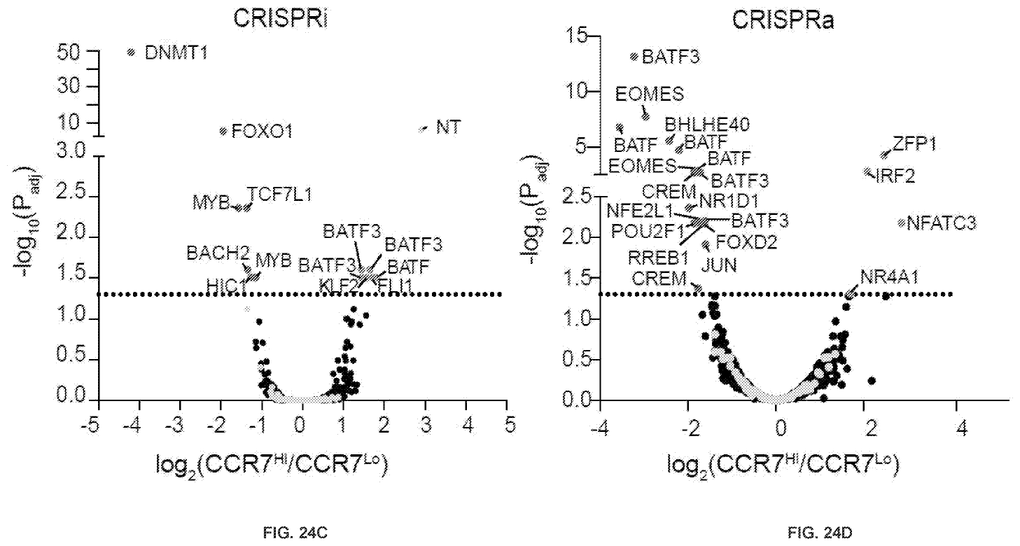

TFs and epigenetic modifiers coordinate complex transcriptional networks that determine cell fate and function. We reasoned that we could identify TFs and epigenetic modifiers that regulate CD8 T cell fate and function by applying high-throughput CRISPRi and CRISPRa screens in primary human T cells. To compile a curated list of TFs associated with T cell state and function, we selected 110 TFs based on motif enrichment in differentially accessible chromatin across T cell subsets (Krishna, S. et al. Science (New York, N.Y.) 2020, 370, 1328-1334; Philip, M. et al. Nature 2017, 545, 452-456; Galletti, G. et al. Nature Immunology 2020; Pritykin, Y. et al. Mol. Cell 2021, 81, 2477-2493 e2410). We then manually added the following 11 transcriptional and epigenetic regulators associated with T cell state: BACH2, TOX, TOX2, PRDM1, KLF2, BMI1, DNMT1, DNMT3A, DNMT3B, TET1, and TET2 for a total of 121 candidate genes. Based on our characterization of dSaCas9-based epigenome editors, we included all specific gRNAs (with a NNGRRT PAM) within a 1,000 bp window centered around the TSS of each gene in our gRNA library. Apart from PBX2 which did not have any gRNAs, the remaining 120 genes were represented by at least 7 gRNAs with an average of 16 gRNAs per gene (FIG. 23A). We included 120 non-targeting gRNAs as negative controls, bringing the final gRNA library to 2,099 gRNAs (FIG. 24B). We cloned the gRNA library into all-in-one CRISPRi and CRISPRa lentiviral plasmids. Subsequent lentiviral titrations revealed a dose-dependent response to the quantity of lentivirus with both CRISPRi and CRISPRa constructs eclipsing 90% transduction rates (FIG. 23B).

We selected CCR7 as a selection readout for our screen, as it is a well-characterized memory T cell surface marker highly expressed in less differentiated T cell subsets including naïve, stem-cell memory, and central memory T cells. We sorted and transduced CD8⁺CCR7⁺ T cells from 2-3 donors with either the CRISPRi or CRISPRa gRNA library. We expanded the cells for 10 days post-transduction to allow enough time for both perturbation of the target gene and any downstream effects on gene regulatory networks, and then sorted transduced cells based on expression of CCR7 (FIG. 24A).

The CRISPRi screen recovered many canonical master regulators of memory T cells including FOXO1⁴⁵, MYB⁴⁶, and BACH2⁴⁷—all of which when silenced led to reduced expression of CCR7, indicative of T cell differentiation towards effector T cells (FIG. 24C). Interestingly, the most significant hit from the CRISPRi screen was DNMT1, which encodes for a DNA methyltransferase that maintains DNA methylation across cell divisions via recognition of hemi-methylated DNA. Genetic disruption of both TET2 and DNMT3A, which encode for proteins that regulate DNA methylation in opposite directions, can improve the therapeutic potential of T cells. Widespread changes in DNA methylation are known to accompany T cell differentiation with accumulation of DNA methylation at promoters of naïve and memory-associated genes and progressive loss of DNA methylation at effector-associated genes. Silencing DNMT1 therefore might accelerate demethylation and transcriptional activation of effector-associated genes that repress CCR7. There was a single non-targeting gRNA (1/120) hit in the CRISPRi screen. The same non-targeting gRNA emerged as a hit in multiple screens using CCR7 as the readout, suggesting a real off-target effect.

The CRISPRa screen also identified many transcription factors that have been implicated in CD8+ T cell differentiation and function such as EOMES, BATF, and JUN (FIG. 24D). Multiple gRNAs targeting the AP-1/ATF transcription factors BATF and BATF3 were enriched in reciprocal directions across CRISPRi and CRISPRa screens, highlighting the power of coupling loss- and gain-of-function perturbations. Interestingly, BATF overexpression in mouse CAR-T cells improves in vivo anti-tumor response, however, BATF overexpression in human CAR-T cells alone does not confer the same therapeutic benefit. In human CAR-T cells, BATF overexpression only improved antitumor response when paired with TFAP4 overexpression. This underscores the significance of species-specific outcomes and the importance of evaluating promising gene candidates in human T cells.

### Example 9

### SingleCell Characterization of Perturbation to Transcriptional and Epigenetic Regulators of T Cell State

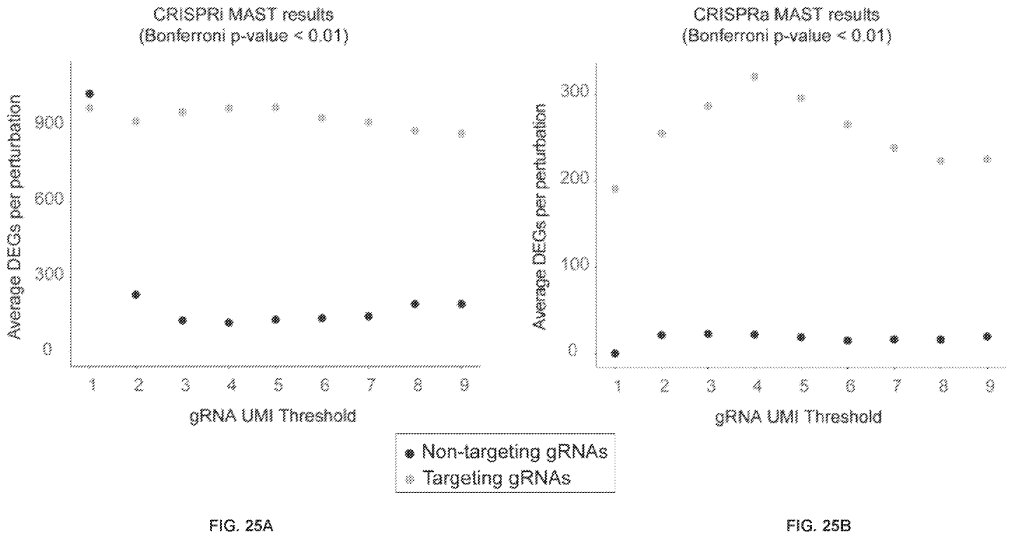

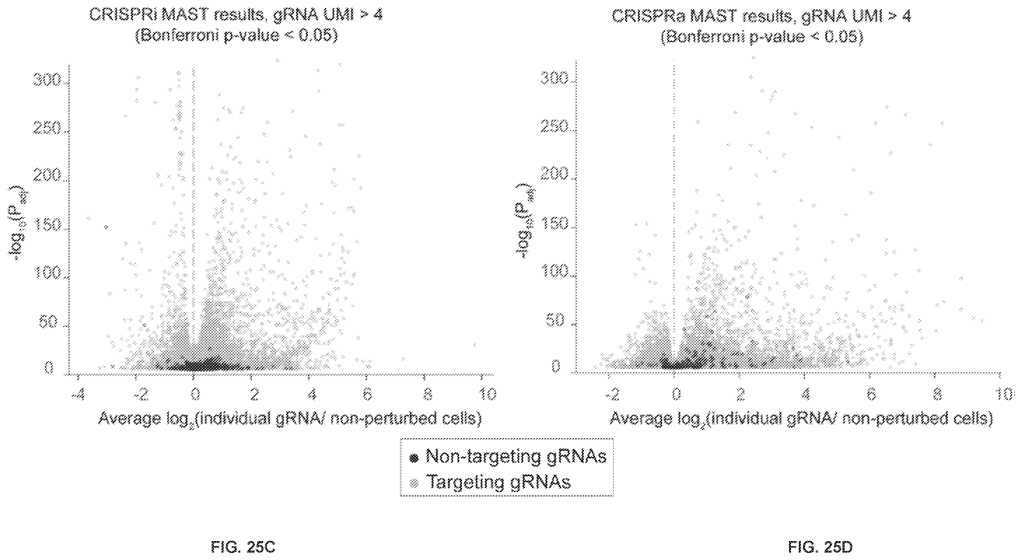

We next characterized the transcriptomic effects of each candidate gene identified from our CRISPRa and CRISPRi screens using single cell RNA-seq (scRNA-seq). All 32 gRNA hits and 8 non-targeting gRNAs were cloned into both CRISPRi and CRISPRa plasmids. We followed the same experimental timeline as the flow-based screens for scRNA-seq characterization, but instead of sorting cells on CCR7 expression, we profiled the transcriptomes and gRNA identity of ˜60,000 cells across three individual donors for each screen. After filtering for high-quality, gRNA-assigned cells, we aggregated the cells across donors and compared the transcriptomes of cells with the same perturbation to non-perturbed cells (cells with only non-targeting gRNAs). As a quality control metric, we compared the quantity and magnitude of effects between targeting and non-targeting gRNAs. Targeting gRNAs were associated with significantly more differentially expressed genes (DEGs) and these gRNA-to-gene links had larger effect sizes than non-targeting gRNAs (FIGS. 25A-25D). Therefore, we proceeded to evaluate the transcriptomic effects of perturbation to each candidate gene.

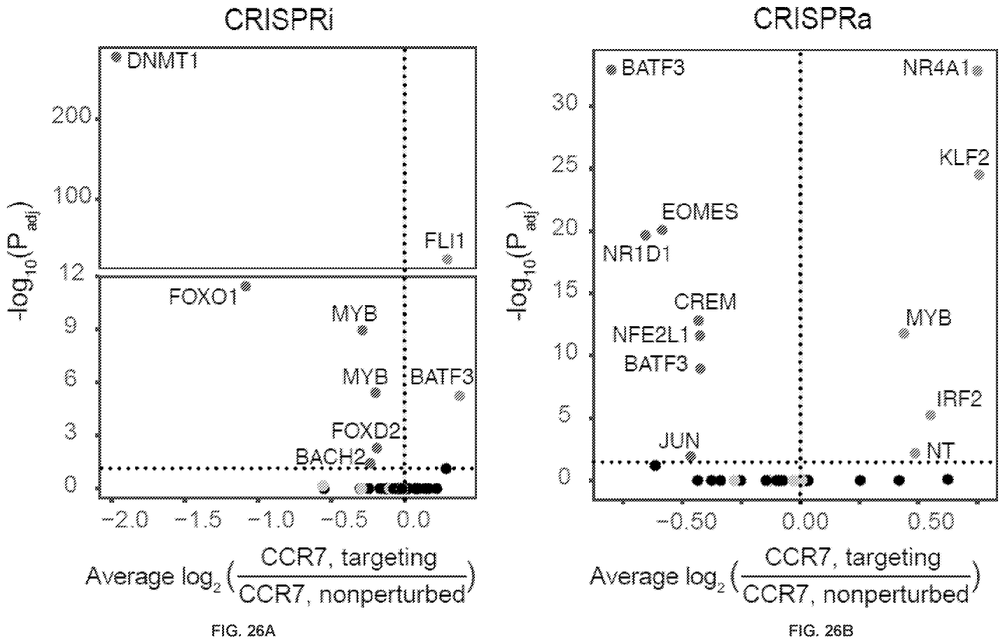

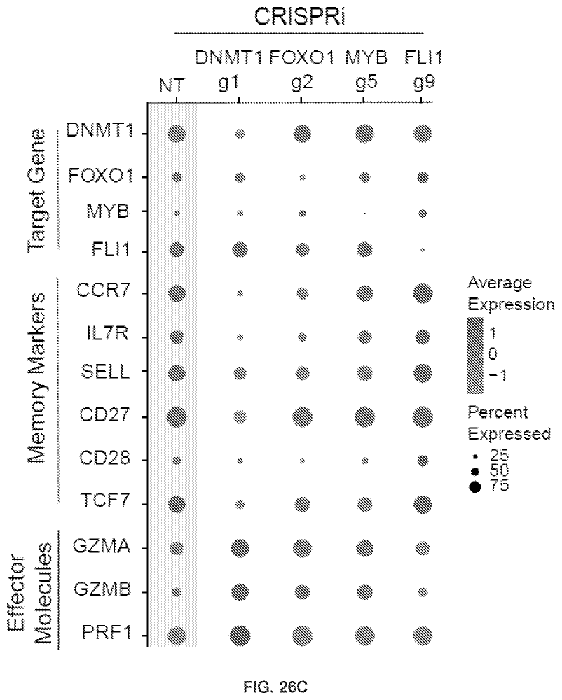

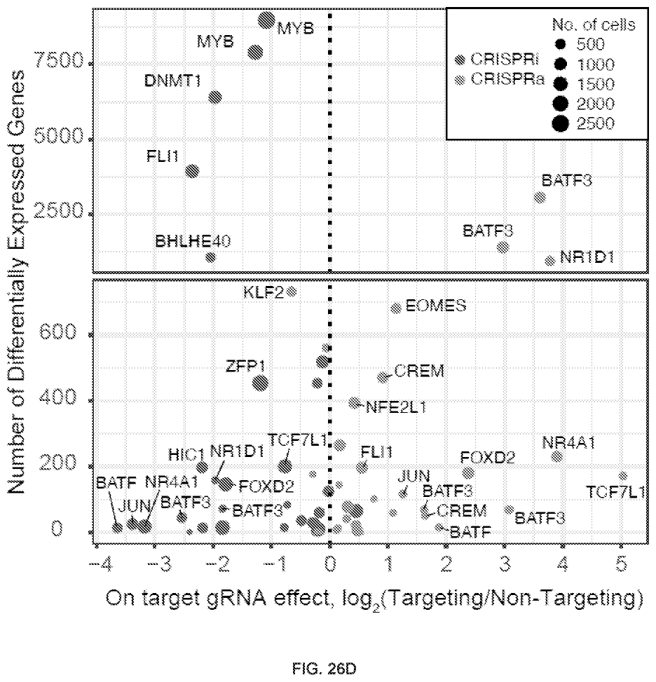

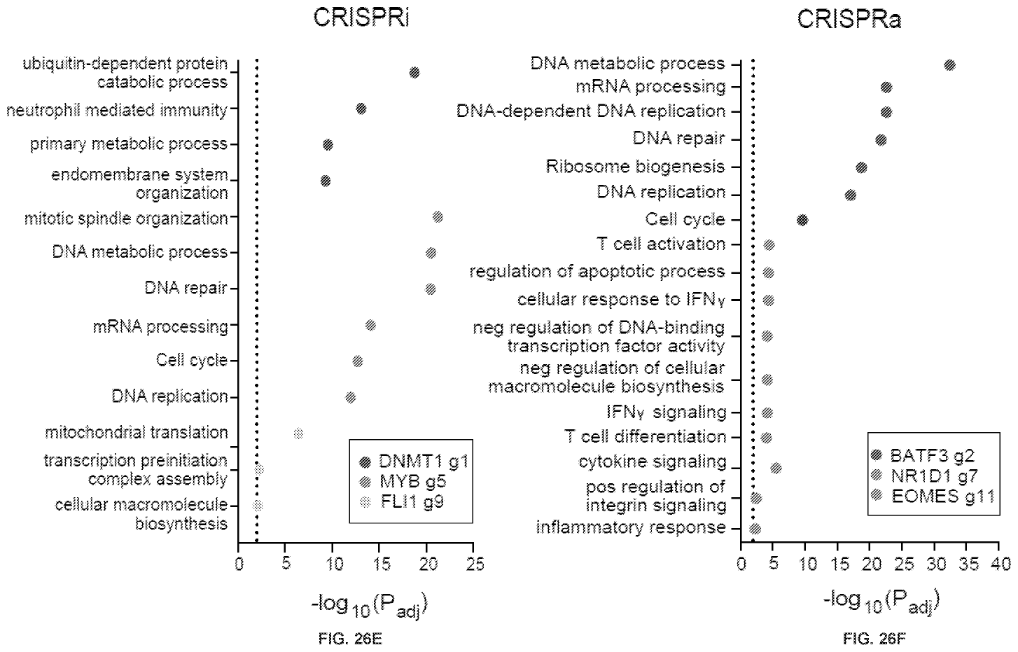

First, we assessed CCR7 expression and found that perturbations affected CCR7 expression in the direction indicated by our flow cytometry-based screens (FIGS. 26A-26B). As CCR7 was selected as a surrogate marker for a memory phenotype, we expected some perturbations to regulate gene expression programs that define T cell subsets. Indeed, scRNA-seq revealed that silencing the top predicted positive regulators of memory (DNMT1, FOXO1, MYB) led to decreased expression of CCR7 and many other memory-associated genes (IL7R, SELL, CD27, CD28, TCF7) and increased expression of effector-associated genes (GZMA, GZMB, PRF1)(FIG. 26C). Conversely, silencing FL/I1 led to increased expression of memory-associated genes. Next, we confirmed on-target activity by evaluating the expression of the target gene for each gRNA perturbation compared to non-perturbed cells. Of CRISPRi and CRISPRa gRNAs assigned to at least 5 cells, 55/60 (92%) gRNAs silenced or activated their gene target, respectively (FIG. 26D). Finally, we examined all DEGs associated with each perturbation to gain an unbiased view of the transcriptomic effects of silencing and activating each gene. Endogenous regulation of several TFs and epigenetic-modifying proteins had widespread transcriptional effects with 12 perturbations (6 CRISPRi and 6 CRISPRa gRNAs) altering expression of >500 genes (FIG. 26D). Unsurprisingly, silencing DNMT1—a global epigenetic modifier—massively altered the transcriptome with 6,401 DEGs and affected general biological processes such as metabolism, endomembrane system organization, and mitotic spindle organization (FIG. 26E).

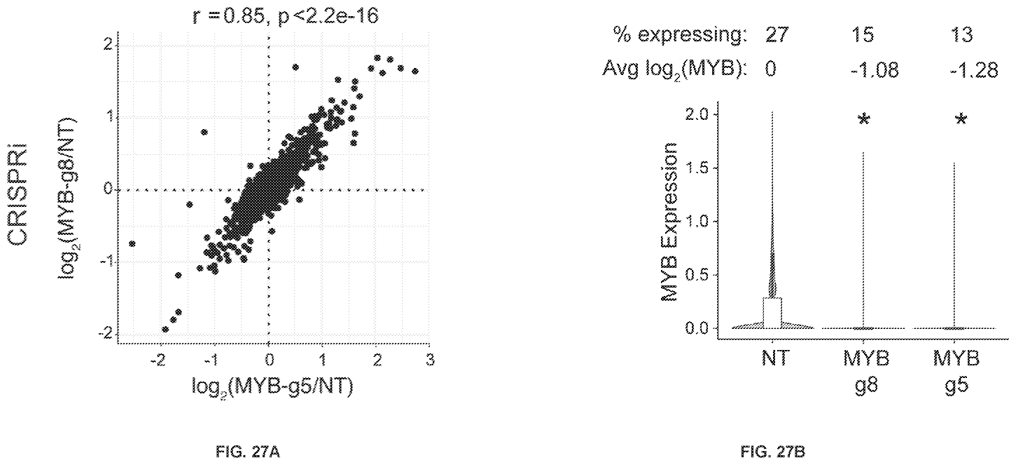

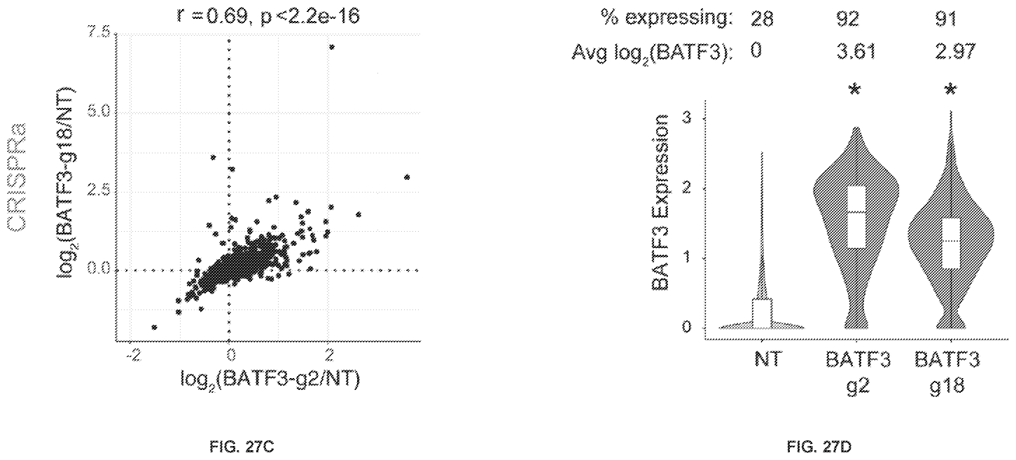

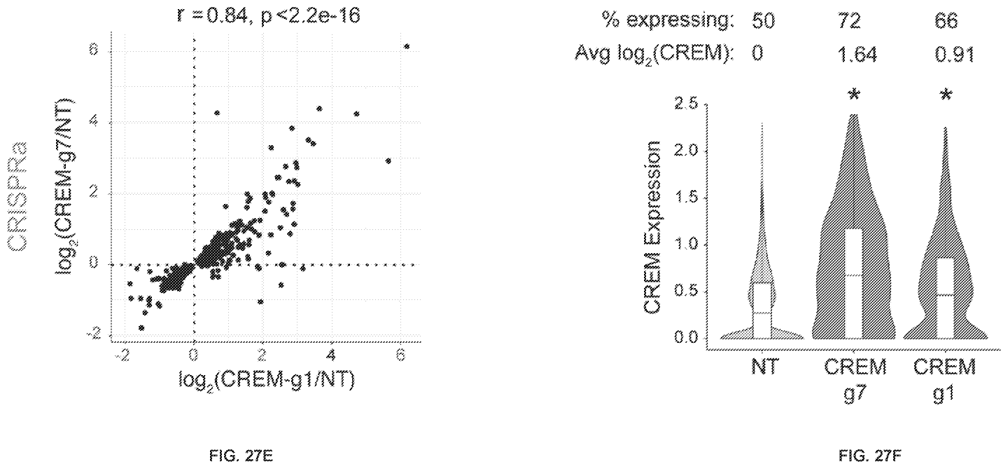

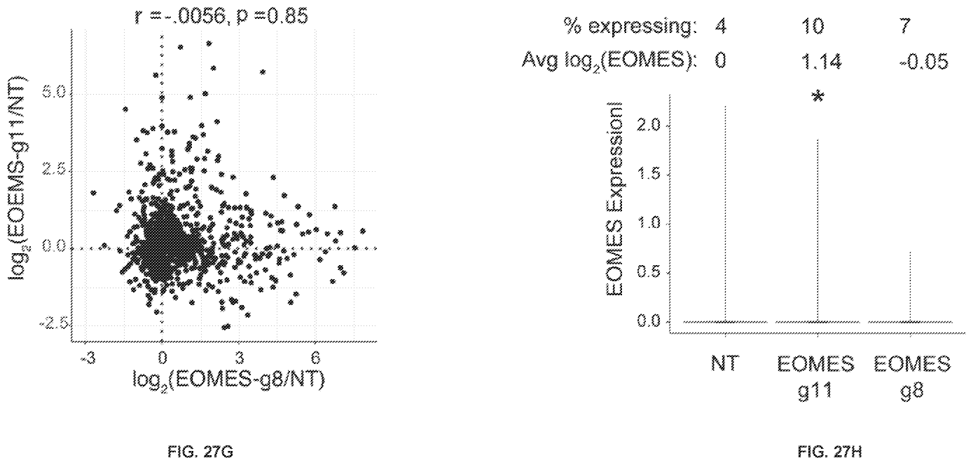

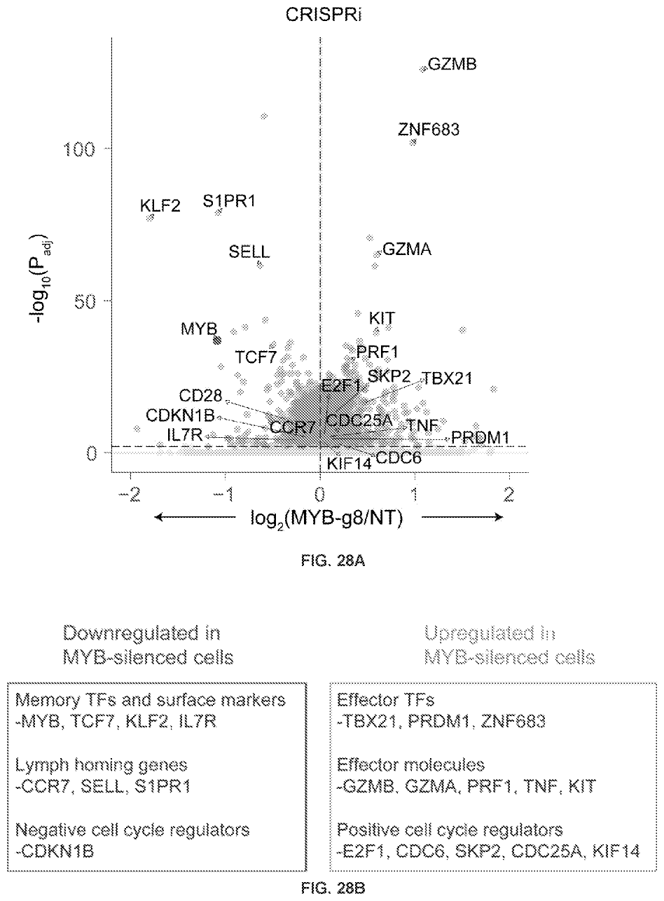

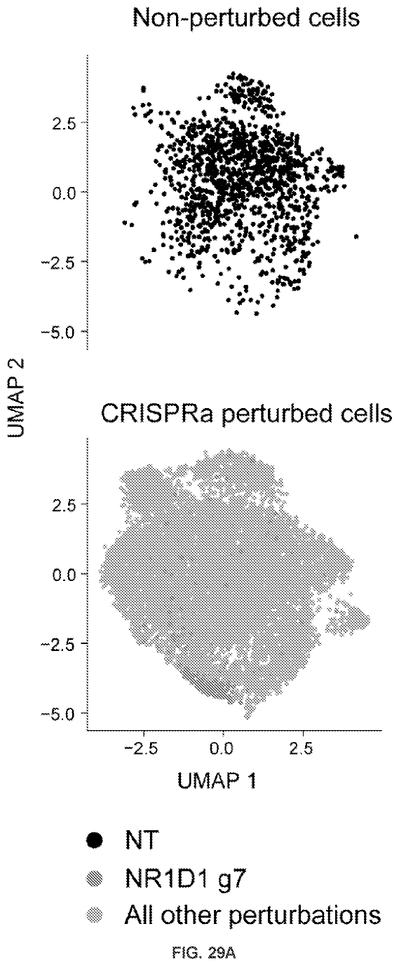

Interestingly, CRISPRi-based repression of MYB with two unique gRNAs resulted in widespread and concordant gene expression changes with 8,976 and 7,899 DEGs (FIGS. 27A-27D). Mouse models of acute and chronic infection have implicated MYB as an essential positive regulator of stem-like memory CD8+ T cells and a small and distinct CD62L+ precursor of exhausted T cell population. In both contexts, MYB-deficient CD8+ T cells lacked therapeutic potential due to impaired recall response or the inability to respond to checkpoint blockage. An important and lingering question has been whether MYB plays a similar role in human CD8+ T cells. Our scRNA-seq data revealed that MYB does indeed regulate human CD8+ T cell stemness with MYB silencing driving CD8+ T cells towards terminal effector T cells. MYB silencing led to downregulation of memory-associated TFs (TCF7, KLF2), lymph homing molecules (CCR7, CD62L, S1PR1), and cell-cycle inhibitors (CDKN1B). In addition, MYB-silenced cells had increased expression of effector-associated TFs (TBX21, PRMD1, ZNF683), effector molecules (GZMB, PRF1), inflammatory cytokines (IFNG, TNF), and positive cell-cycle regulators (E2F1, CDC6, SKP2, CDC25A and KIF14) (FIGS. 26E and 28A-28B). The two MYB CRISPRi gRNAs were represented by the first and third most cells across both CRISPRi and CRISPRa screens, suggesting that MYB silencing promoted T cell proliferation (FIG. 26D).

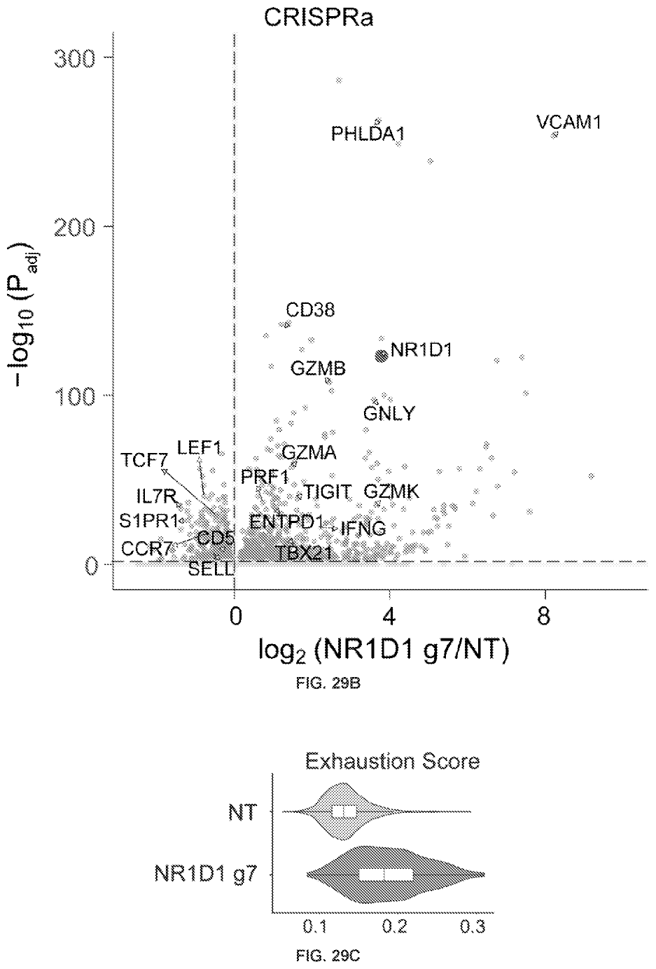

Coupling CRISPR-based gene activation with a scRNA-seq readout enabled causal inference of gene function in human CD8 T cells. Endogenous activation of several TFs including NR1D1, EOMES, and BATF3 had large effects on T cell state. Perturbation-driven single cell clustering revealed a distinct cluster with NR1 D1 activation (FIG. 29A). NR1D1 encodes a nuclear receptor subfamily 1 transcription factor and negatively regulates expression of core clock proteins that govern cyclical gene expression patterns. Integrative analysis of bulk ATAC-seq data across 12 independent studies of CD8 T cell dysfunction in cancer and infection found that the NR1D1 motif was enriched in open chromatin of exhausted T cells. The causal role of NR1 D1 in CD8 T cells, however, has not been studied. NR1 D1 activation resulted in 939 differentially expressed genes with 646 upregulated and 293 downregulated genes (FIG. 29B). In agreement with NR1 D1 motif enrichment, a large set of effector and exhaustion-associated genes were markedly upregulated with NR1D1 activation. To better understand the magnitude of exhaustion induction by NR1D1, we calculated an exhaustion gene signature score using a defined set of 82 exhaustion-specific genes (Zheng, C. et al. Cell 2017, 169, 1342-1356). NR1D1-perturbed cells had a significantly higher exhaustion gene signature score than non-perturbed cells and all other CRISPRa perturbations (FIG. 29C). Consistent with NR1 D1 inducing T cell exhaustion, many memory-associated surface markers (IL7R, CCR7, SELL, CD5) and TFs (TCF7, LEF1) were downregulated. Overall, NR1D1 activation ‘synthetically’ induced an exhausted transcriptomic profile in the context of acute stimulation.

Endogenous activation of EOMES, a master regulator of effector T cells, drove cytokine signaling and inflammatory response, but did not lead to an increase in exhaustion-related genes (FIG. 26F). The top two BATF3 gRNA hits from our flow based CRISPRa screen had strong and concordant effects with 3,056 and 1,402 DEGs (FIG. 26D and FIGS. 27E-27H). Gene ontology analyses revealed that BATF3-induced genes were enriched for DNA and mRNA metabolic processing, ribosomal biogenesis, and cell-cycle pathways, suggesting that BATF3 improves T cell fitness (FIG. 26F).

### Example 10

### BATF3 Overexpression Programs Memory-Like Features and Attenuates Effector and Exhaustion Gene Programs

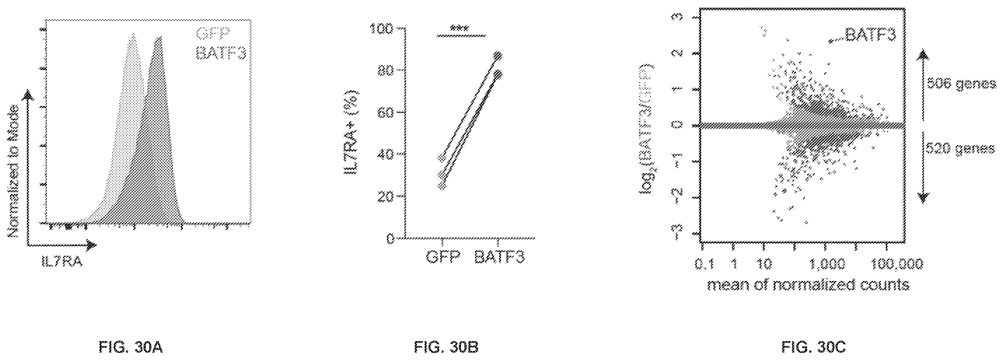

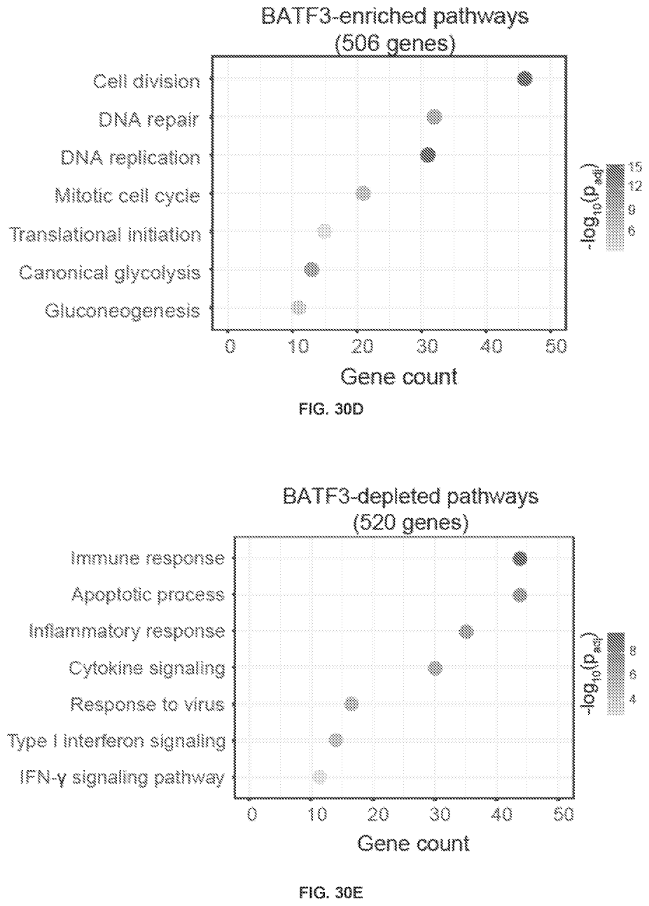

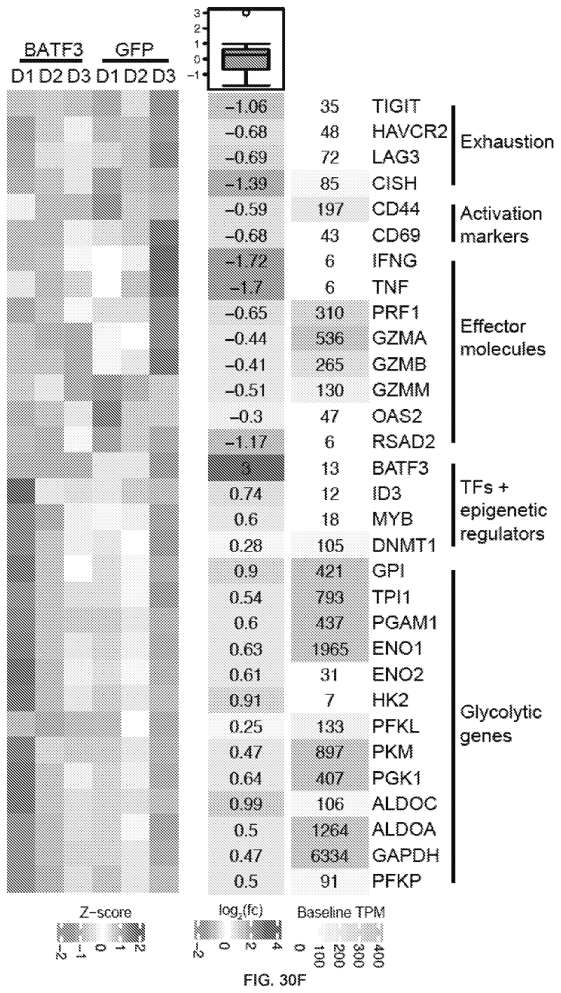

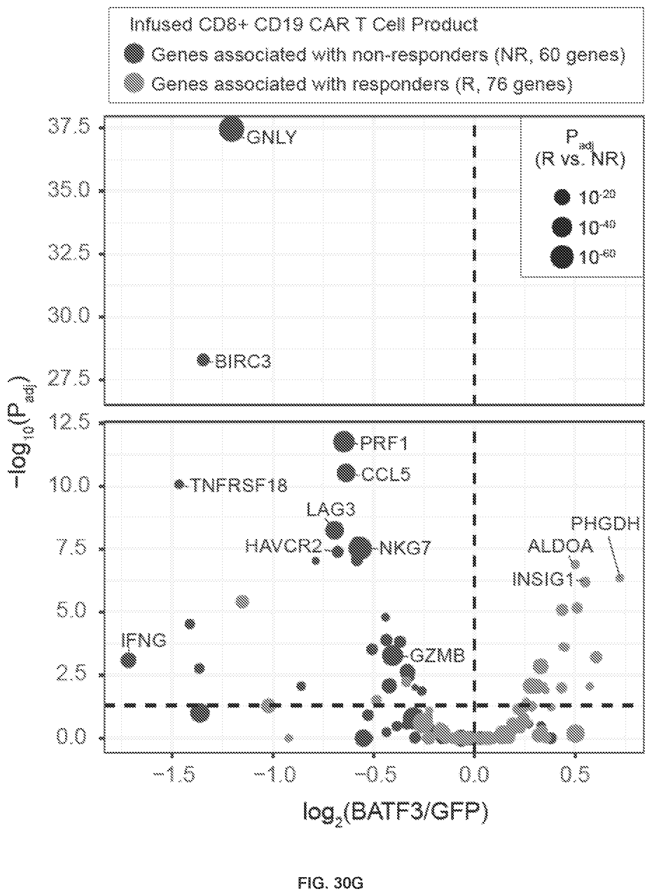

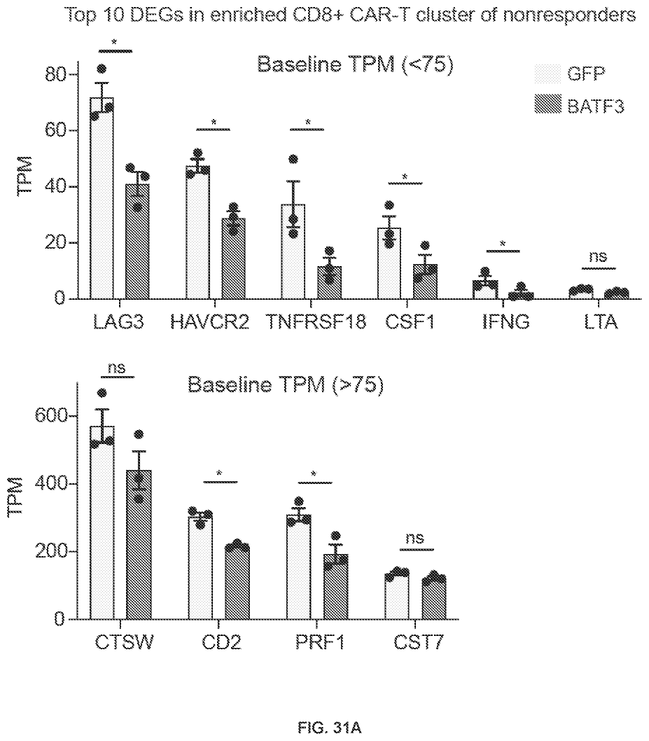

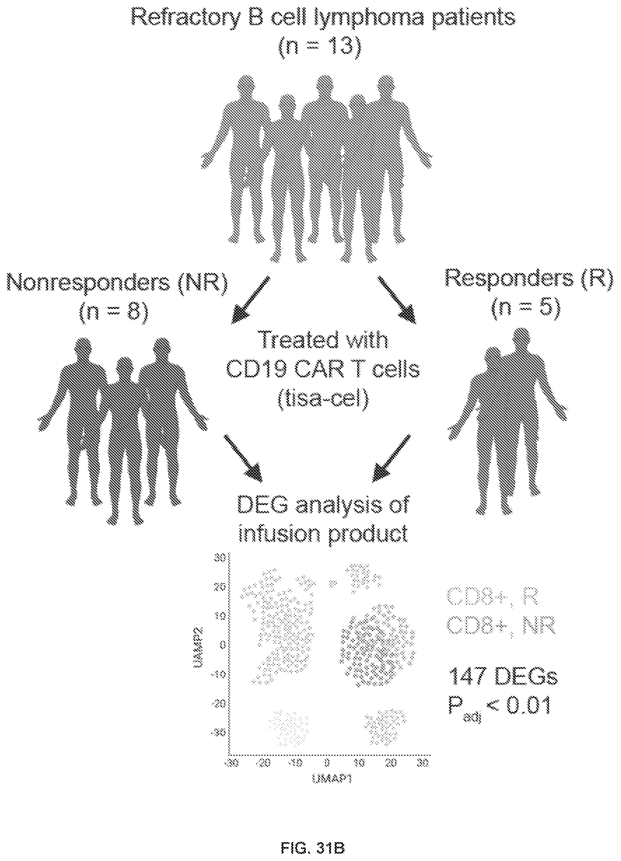

In murine models of viral infection, BATF3 programs a memory T cell phenotype and BATF3 deficiency negatively impacts CD8 T cell proliferation and recall response. However, the molecular and functional role of BATF3 in human T cells has not been well defined. Studies of other master regulators of T cell state, such as TOX and BATF, have illustrated the potential lack of concordance in gene function and phenotypic effect between murine and human T cells. To examine the effects of BATF3 in human CD8+ T cells, we ectopically expressed BATF3 or GFP (control) from lentiviral vectors and performed immunophenotyping, RNA-sequencing, ATAC-sequencing, and functional assays. First, we found that BATF3 overexpression markedly increased expression of IL7R, a surface marker associated with T cell survival, long-term persistence, and positive clinical response to ACT (FIGS. 30A-30B). Next, we applied RNA-seq to gain an unbiased view of the transcriptomic changes induced by BATF3 overexpression. Compared to control cells, there were over 1,000 DEGs distributed almost equally between upregulated and downregulated genes (FIG. 30C). To gain insight into the biological processes affected by BATF3 overexpression, we passed upregulated and downregulated DEGs into the DAVID functional annotation database (FIGS. 30D-30E). Many BATF3-enriched pathways were related to T cell proliferation such as cell division, DNA replication, and mitotic cell cycle. Consistent with this functional annotation, BATF3 overexpression is known to increase T cell proliferation across diverse stimulation conditions. Moreover, BATF3 increased expression of genes related to translation and glycolysis (FIG. 30F). The mobilization of glycolytic enzymes is critical for rapid recall response in central memory T cells, suggesting that BATF3 overexpression could improve killing capacity upon restimulation. In line with BATF3 enforcing a memory-like transcriptional profile, BATF3 dampened effector programs with downregulation of activation markers (CD44, CD69), inflammatory cytokines (IFNG, TNF), and cytotoxic molecules (PRF1, GZMA, GZMB)(FIG. 30F). A recent study found that the infused CD19-targeting CAR-T cell product of non-responders had a significantly higher proportion of CD8+ T cells in a cytotoxic or exhausted phenotype than responders. Notably, BATF3 significantly decreased expression of seven of the top ten differentially expressed genes defining this non-responder cluster, including cytotoxic (PRF1, IFNG) and exhaustion genes (LAG3, HAVCR2, TNFRSF18)(FIG. 31A). This finding prompted us to systematically identify DEGs between the infused CD8+CD19 CAR-T cell product of responders and non-responders (FIG. 31B). There were 147 DEGs between CD8+ T cells of responders and non-responders in this public dataset. We then examined expression of these DEGs from our bulk RNA-seq data with BATF3 overexpression. Of the 147 DEGs, 136 genes were detected in our RNA-seq data. Strikingly, BATF3 programmed a gene signature that strongly correlated with positive clinical outcomes as BATF3 silenced 45% (27/60) of genes associated with non-responders and activated 28% (21/76) of genes associated with responders (FIG. 30G). Only 3.7% (5/136) of genes were regulated in a direction opposing positive clinical response, providing further evidence that BATF3 drives a transcriptional signature associated with positive clinical outcomes.

### Example 11

### BATF3 Remodels the Epigenetic Landscape of CD8+ T Cells

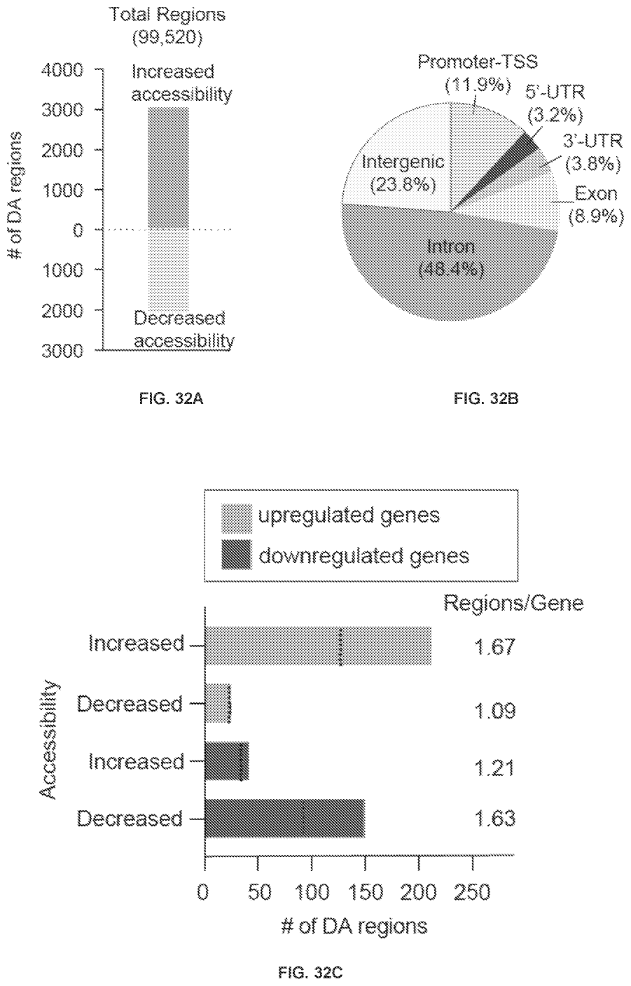

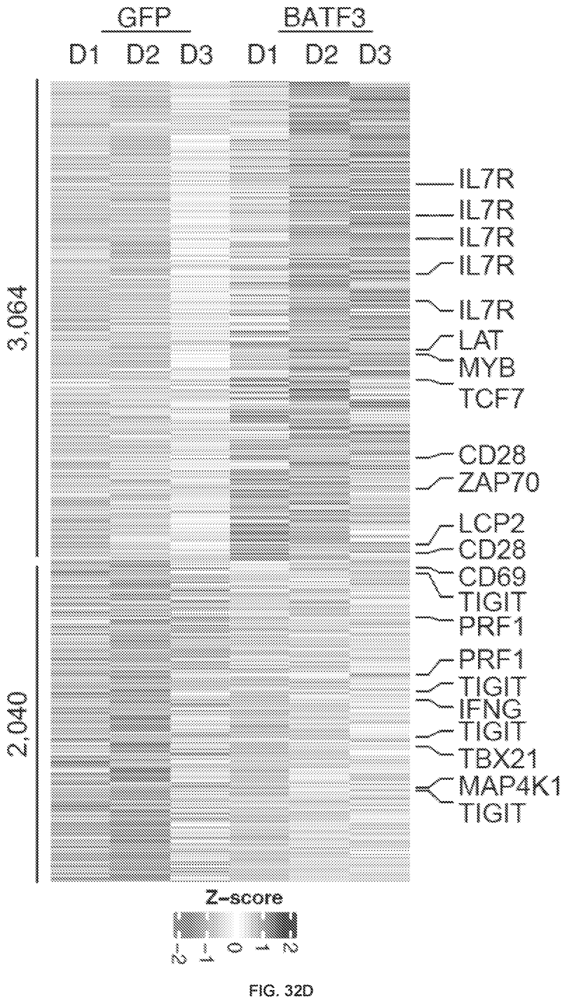

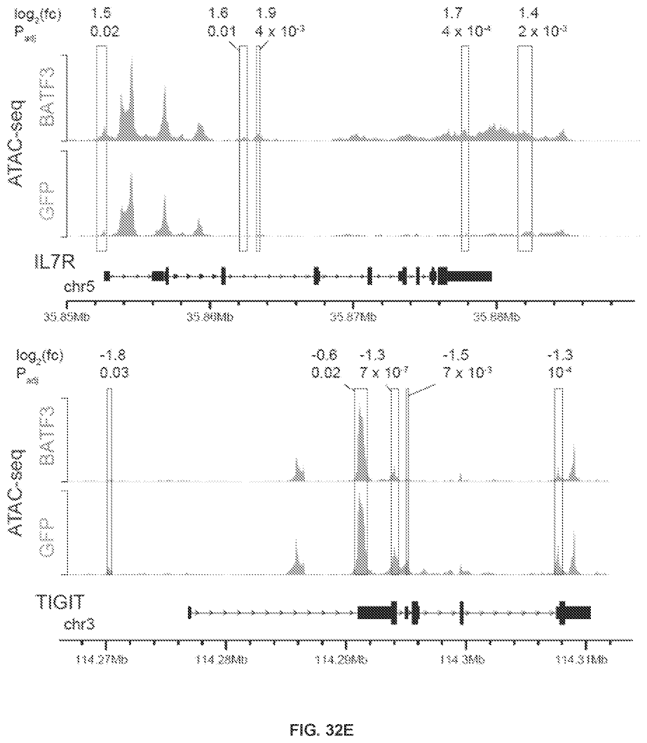

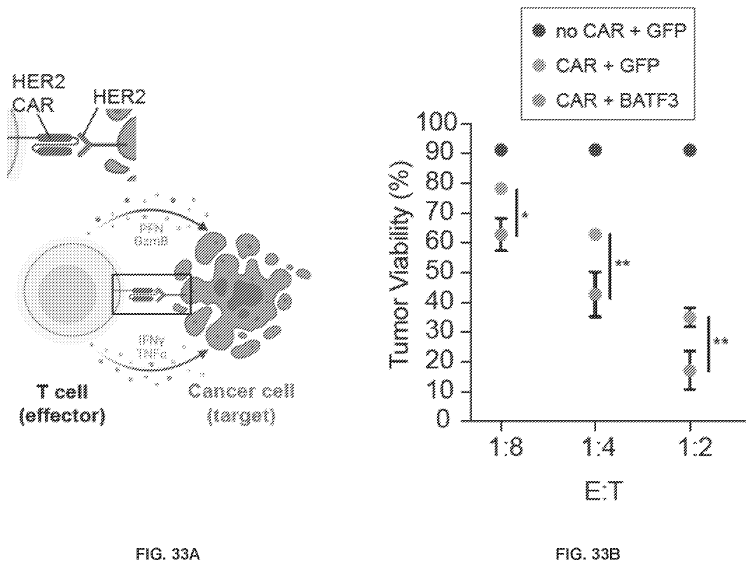

To assess remodeling of the epigenetic landscape in response to BATF3, we performed ATAC-seq on control and BATF3-overexpressing T cells. There was extensive chromatin remodeling with 5,104 differentially accessible regions between the groups (FIG. 32A). Of these regions, roughly 60% were more accessible with BATF3 overexpression. Most of these changes were in intronic or intergenic regions consistent with cis-regulatory or enhancer elements (FIG. 32B). To better understand whether changes in chromatin accessibility corresponded to changes in gene expression, we jointly analyzed our ATAC-seq and RNA-seq data. First, we assigned each differential region to its closest gene to estimate genes that could be regulated in cis by these elements. We then quantified how many differential regions proximal to DEGs gained or lost accessibility. There was an enrichment of regions with increased or decreased accessibility proximal to upregulated and downregulated genes, indicative that BATF3-induced epigenetic changes affected transcriptional programs (FIG. 32C). About ˜⅕ of the genes that changed transcriptionally were associated with a corresponding differentially accessible region (219 out of 1,026 genes).

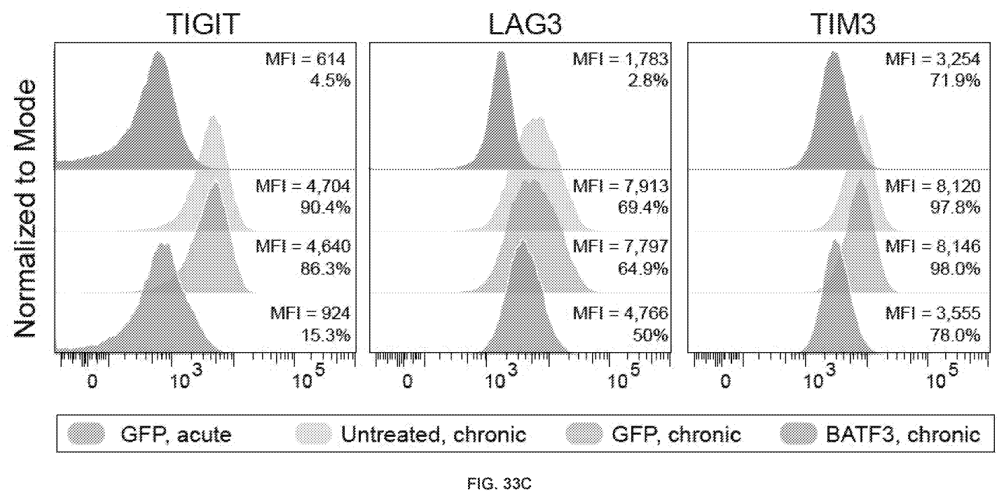

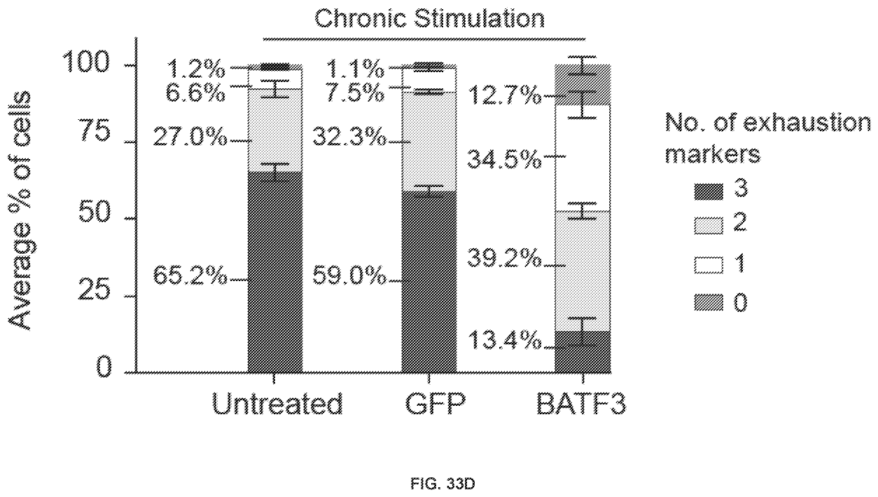

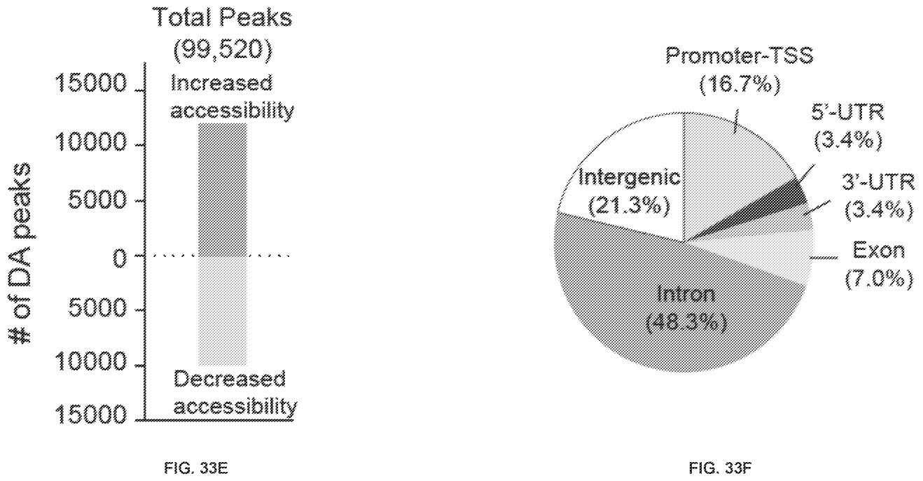

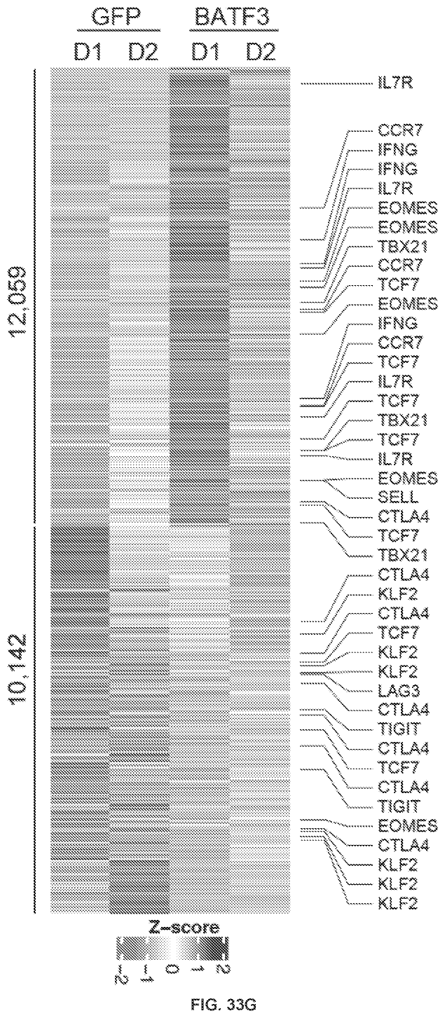

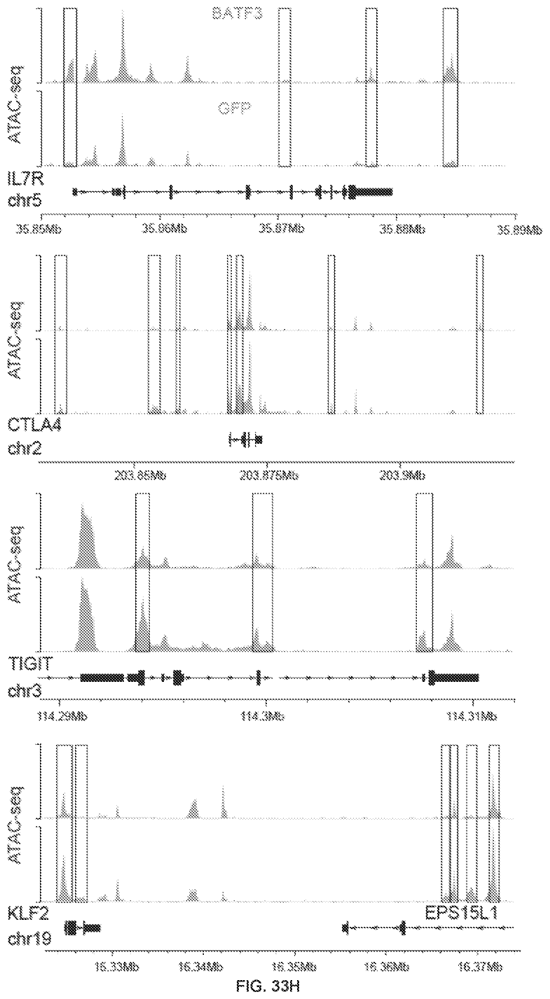

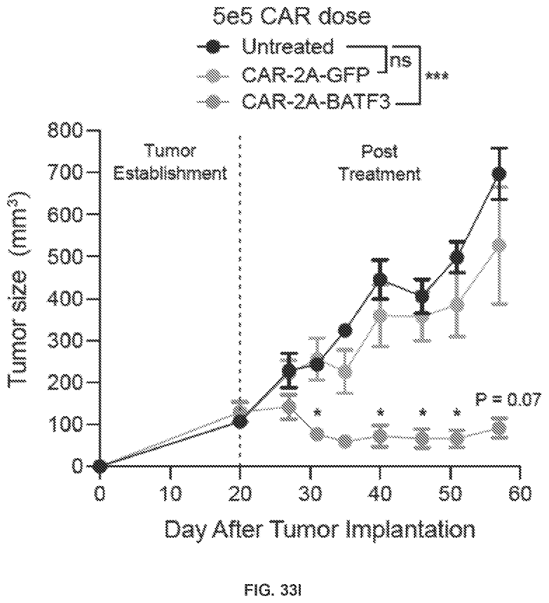

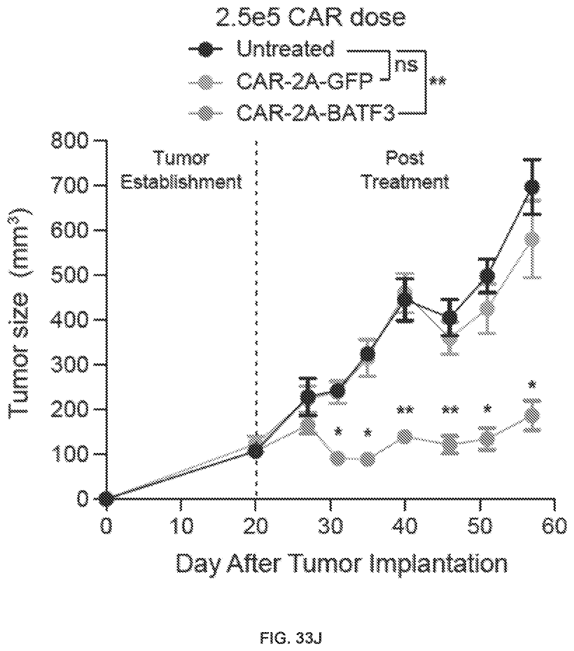

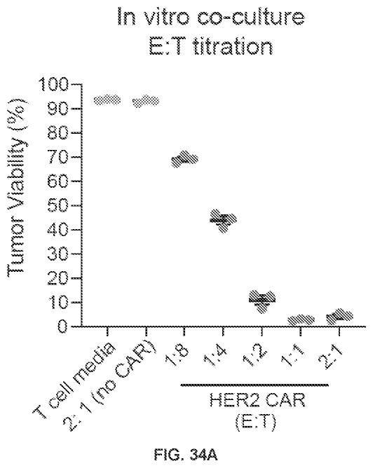

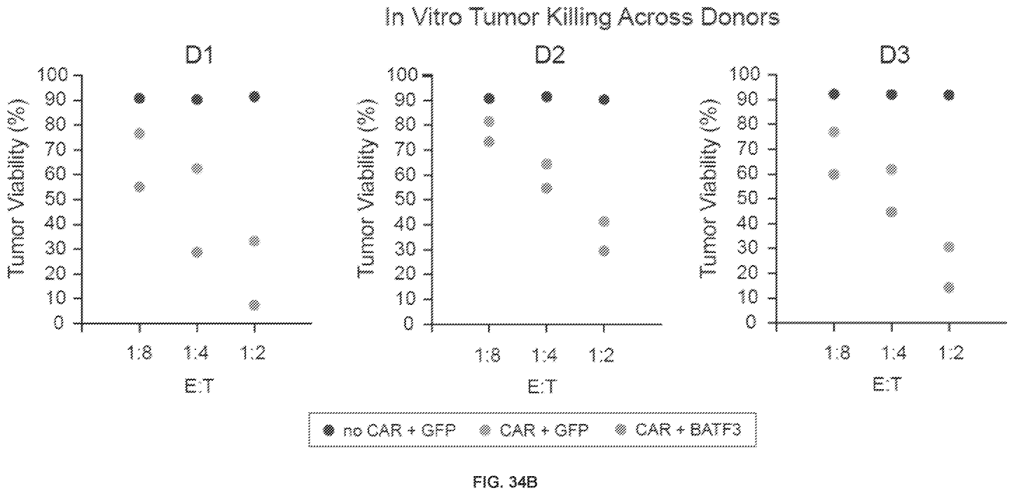

Albeit less frequently, there were examples of more accessible chromatin regions near downregulated genes and less accessible chromatin regions near upregulated genes. As many genes use functionally redundant enhancers to buffer gene expression against mutations, it is likely that changes in accessibility to a single cis-regulatory or enhancer element are not always sufficient to change gene expression. Indeed, we observed that DEGs with supporting changes in chromatin accessibility had on average more differential regions than DEGs with opposing changes in chromatin accessibility (FIG. 32C). For example, BATF3 extensively remodeled the chromatin landscape at IL7R and TIGIT (FIGS. 32D-32E). BATF3 increased accessibility at the IL7R promoter, intronic, 3′-UTR, and intergenic regions and decreased accessibility at distal intergenic, 5′-UTR, and exonic regions of TIGIT, consistent with immunophenotyping and RNA-seq (FIGS. 30A-30B, 30F, 33C). There were also several examples where increased or decreased chromatin accessibility did not lead to corresponding transcriptional changes. Although there were no transcriptional changes, BATF3 increased chromatin accessibility near genes encoding for LAT, ZAP70, LCP2, and CD28, all of which are strong positive regulators of IFNG and IL-2 and decreased chromatin accessibility near MAP4K1, a potent negative regulator of IFNG and IL-2. These regions may be programmed to a poised state, and additional stimuli or factors in combination with BATF3 may be required to activate expression of these genes.

### Example 12

### BATF3 Overexpression Improves Tumor Control and Counters T Cell Exhaustion

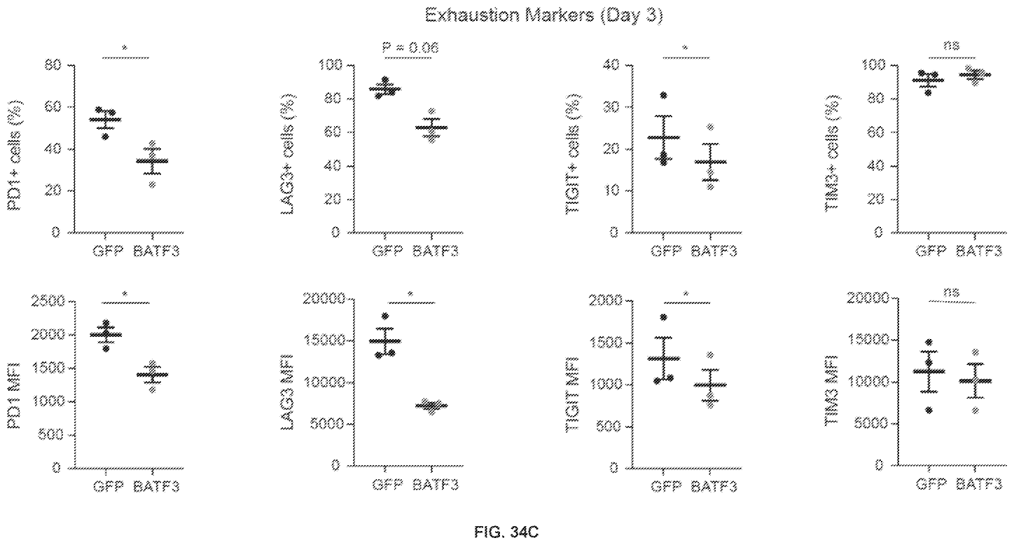

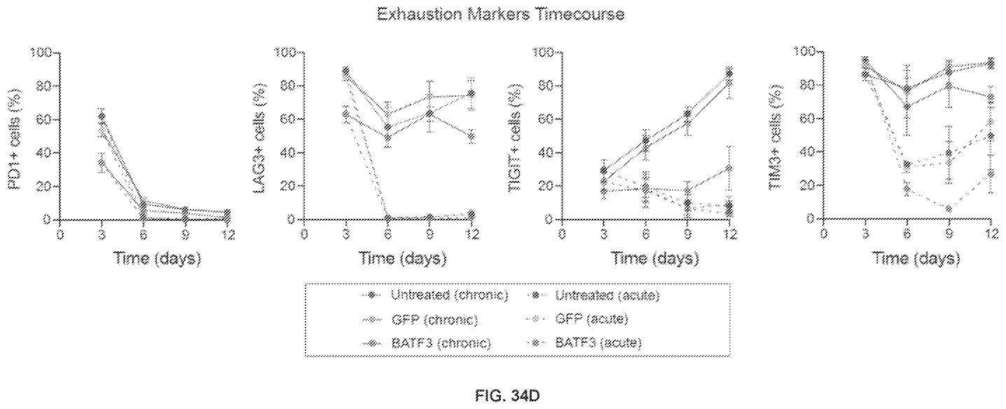

Given the widespread molecular effects of BATF3 overexpression, we hypothesized that BATF3 might improve T cell function. First, we evaluated the antitumor capacity of BATF3 overexpressing T cells using an in vitro human epidermal growth factor 2 (HER2)-positive tumor model (FIG. 33A). We serially transduced CD8+ T cells from three donors with lentiviruses encoding for HER2 CAR and either GFP or BATF3. We expanded the cells for 12 days to ensure the cells were no longer in a hyperactive state from the initial stimulation and to provide sufficient time for BATF3-mediated effects. We then co-cultured a human HER2+ breast cancer line (SKBR3 s) with wild type, HER2 CAR+, and HER2 CAR+BATF3+ T cells. There was no tumor killing in the absence of CAR T cells (FIG. 34A). Strikingly, CAR T cells co-expressing BATF3 were more potent tumor killers than control CAR T cells across donors and effector:target (E:T) ratios (FIG. 33B and FIG. 34B).

We then investigated whether BATF3 could improve T cell fitness in the context of chronic antigen stimulation using two in vitro models: CD3/CD28 bead restimulation and restimulation with tumor cells. We measured expression of exhaustion-associated markers (PD1, TIGIT, LAG3, TIM3) in control and BATF3-expressing T cells after a single CD3/CD28 bead stimulation (acute stimulation) or four rounds of CD3/CD28 bead stimulation (chronic stimulation). As previously observed, PD1 expression peaked after the initial stimulation and then tapered off over time, whereas TIGIT, LAG3, and TIM3 expression were maintained or increased after each subsequent round of stimulation (FIGS. 34C-34D). Notably, BATF3 attenuated the extent of PD1 induction and reduced expression of TIGIT, LAG3, and TIM3 with a profile more closely resembling acutely stimulated T cells (FIG. 33C and FIGS. 34C-34D). As terminally exhausted T cells are often defined by co-expression of multiple exhaustion-associated markers, we quantified the proportion of cells expressing each combination of TIGIT, LAG3, and TIM3. Only 13% of BATF3 overexpressing T cells co-expressed all three markers compared to 65% and 59% of untreated and GFP T cells (FIG. 33D).

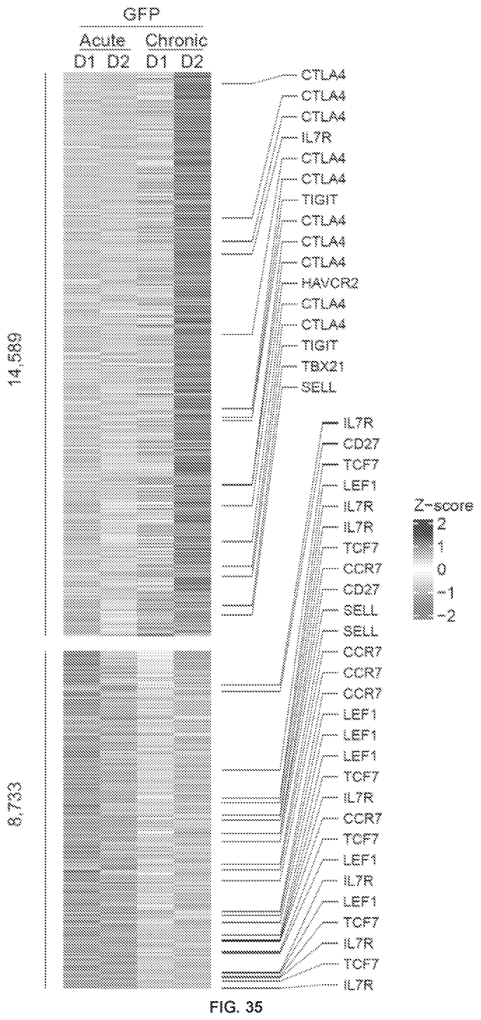

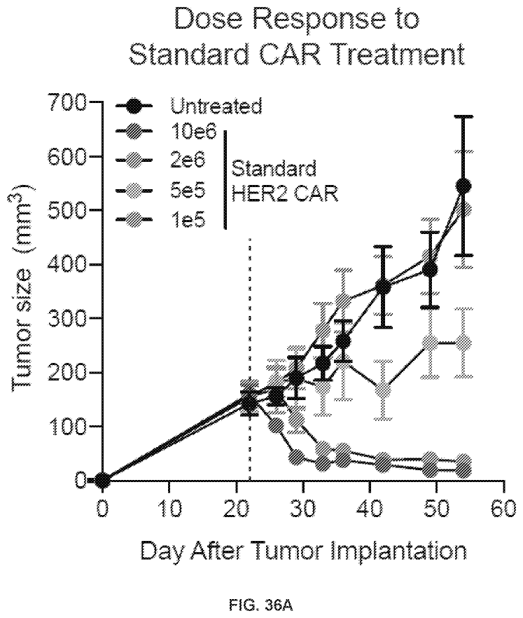

Another defining feature of T cell exhaustion is the accumulation of widespread epigenetic changes that persist independent of chronic antigen stimulation. To recapitulate this feature in vitro, we repeatedly stimulated CAR T cells with tumor cells and measured the effects using ATAC-seq. Indeed, chronic antigen stimulation induced widespread changes in chromatin accessibility with 23,322 differentially accessible regions between acutely and chronically stimulated control cells. Many of these regions were proximal to memory and effector/exhaustion-genes, providing further support for this model (FIG. 35). Next, we evaluated the differences in the epigenetic landscape of chronically stimulated T cell with or without BATF3 overexpression. There were 22,201 differentially accessible regions between control and BATF3 T cells with most regions in intronic and intergenic regions (FIGS. 33E-33F). Interestingly, we observed increased accessibility at regions near both memory (TCF7, MYB, IL7R, CCR7, SELL) and effector-associated genes (EOMES, TBX21, IFNG), which are traditionally silenced in memory T cells (FIGS. 33G-33H). This may represent a hybrid T cell phenotype or heterogenous subpopulations of memory and effector T cells. The latter explanation supports the linear model of T cell differentiation, where a subpopulation of activated memory T cells self-renew, proliferate, and differentiate into short-lived effector T cells. In contrast, we observed reduced accessibility at regions close to exhaustion genes (TIGIT, CTLA4, LAG3), consistent with our CD3/CD28 bead restimulation data.

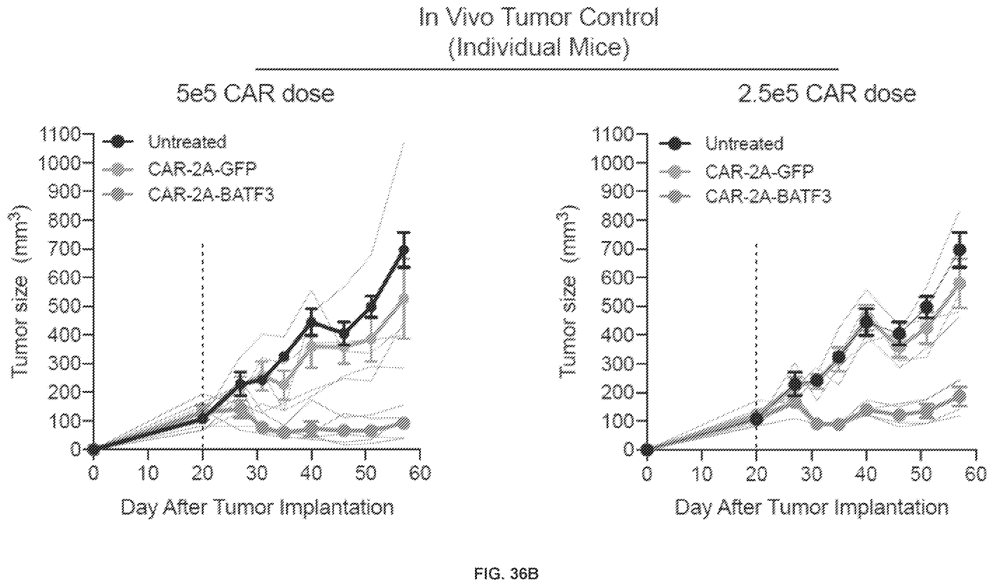

We sought to evaluate the in vivo tumor killing capacity conferred by BATF3 overexpression using a solid tumor model, given the known role of T cell exhaustion in limiting ACT efficacy in solid tumors. We orthotopically implanted a human HER2+ breast cancer line (HCC1954) into immunodeficient NSG mice. After 20 days, we intravenously injected mice with two sub-curative doses (2.5×10⁵ and 5×10⁵) of HER2 CAR T cells with or without BATF3 overexpression (FIGS. 331-33J and 36A-36B). We measured tumor volumes every 4-6 days. BATF3 HER2 CAR T cells markedly enhanced tumor control at each dose (FIGS. 33J-331 and 36B-36B). Notably, the tumor growth of mice treated with the low dose of 2.5×10⁵ control HER2 CAR T cells was completely unrestrained, mimicking that of untreated mice (FIG. 33I). In stark contrast, there was clear regression and delay of tumor growth with the matched dose of BATF3 HER2 CAR T cells, highlighting the functional benefit of BATF3 overexpression. In summary, BATF3 promotes a memory phenotype, counters phenotypic and epigenetic signatures of T cell exhaustion, and improves in vitro and in vivo tumor control.

### Example 13

### Discussion

Described herein is the development and characterization of compact and efficient dSaCas9-based epigenome editors to systematically identify transcriptional and epigenetic regulators of primary human CD8+ T cell state through complementary loss-of-function and gain-of-function CRISPRi/a screens. Although we used a curated 120 gene library for our screens, this technology could readily be scaled to profile all catalogued human genes for their coordination of complex phenotypes. Nevertheless, our CRISPRi/a screens recovered many known and novel regulators of CD8+ T cell state with a striking convergence on BATF3. A prominent effect of BATF3 overexpression was activation of IL7R, which encodes for the IL-7 receptor. A primary reason for lymphodepleting regimens before CAR T cell infusion in clinical protocols is to maximize the availability of homeostatic cytokines (IL-2, IL-7, and IL-15) by eliminating competing immune cells. Increased IL7R expression on engineered T cells therefore might increase their sensitivity to IL-7 signaling and enable lower doses of conditioning lymphodepletion agents, which increase the risk of infection and have other associated toxicities.

BATF3 overexpression improved in vitro and in vivo tumor cell killing and countered canonical signatures of T cell exhaustion. Mechanistically, BATF3 competes with other TFs (such as FOS) for binding to JUN. Unlike JUN-FOS heterodimers, JUN-BATF3 heterodimers can interact with IRF family members and regulate distinct transcriptional programs. Supporting this model, higher ratios of Jun to Fos expression have been associated with improved in vivo tumor control. Overall, the dynamics and combinatorial interactions between AP-1 transcription factors control cellular processes that determine T cell state and function and are therefore promising therapeutic candidates for ACT.

All current FDA approved ACT products rely on lentiviral delivery of CARs or TCRs. Integrating an additional ORF such as BATF3 into CAR/TCR constructs should therefore not significantly alter existing ex vivo manufacturing procedures. The compact size of BATF3 (only 378 bp) should not impede production of high titer lentivirus. For example, transduction rates exceeded 90% with lentivirus encoding for HER2-CAR-2A-BATF3. BATF3 and other promising gene candidates may be transferred into the clinic. The safety of engineered T cells may be safely assessed beforehand. Further measures such as transient delivery of mRNA encoding for the transgene, tuning transgene expression through regulatory elements or genetic circuits, or suicide switches to control the activity of T cells in vivo could be implemented to resolve potential adverse side effects of engineered T cells.

This work expands the toolkit of epigenome editors as well as our understanding of genes that regulate human CD8 T cell state and function. This catalogue of genes could serve as a basis for engineering the next generation of T cell therapies for cancer treatment.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A composition for modulating T cells, the composition comprising a modulator of a gene selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and FLI1.

Clause 2. The composition of clause 1, wherein modulating T cells comprises increasing T cells, or increasing memory T cells, or increasing the lifetime of a T cell, or preventing T cell exhaustions, or reversing T cell exhaustions, or reducing T cell exhaustion, or enhancing the therapeutic potential of T cells, or a combination thereof.

Clause 3. The composition of clause 1 or 2, wherein the modulator comprises a polypeptide, or a polynucleotide, or a small molecule, or a lipid, or a carbohydrate, or a combination thereof.

Clause 4. The composition of clause 3, wherein the modulator comprises an antibody or siRNA or shRNA.

Clause 5. The composition of clause 3, wherein the modulator comprises a DNA targeting composition, the DNA targeting composition comprising: a Cas9 protein or a fusion protein, wherein the fusion protein comprises two heterologous polypeptide domains, wherein the first polypeptide domain comprises a Cas9 protein and the second polypeptide domain has an activity selected from transcription activation activity, transcription repression activity, nuclease activity, transcription release factor activity, histone modification activity, nucleic acid association activity, methylase activity, and demethylase activity; and at least one guide RNA (gRNA) that targets the Cas9 protein to the gene or a regulatory element thereof.

Clause 6. A DNA targeting composition comprising: a Cas9 protein or a fusion protein, wherein the fusion protein comprises two heterologous polypeptide domains, wherein the first polypeptide domain comprises a Cas9 protein and the second polypeptide domain has an activity selected from transcription activation activity, transcription repression activity, nuclease activity, transcription release factor activity, histone modification activity, nucleic acid association activity, methylase activity, and demethylase activity; and at least one guide RNA (gRNA) that targets the Cas9 protein to a target gene or a regulatory element thereof, wherein the target gene is selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and FLI1.

Clause 7. The composition of any one of clauses 5-6, wherein the gRNA is encoded by a polynucleotide comprising a sequence selected from SEQ ID NOs: 57-88, or comprises a sequence selected from SEQ ID NOs: 89-120.

Clause 8. The composition of any one of clauses 5-7, wherein the Cas protein comprises a Streptococcus pyogenes Cas9 protein, or a Staphylococcus aureus Cas9 protein, or any fragment thereof.

Clause 9. The composition of any one of clauses 5-8, wherein the Cas9 protein comprises an amino acid sequence having at least 90% or greater identity to a sequence selected from SEQ ID NOs: 26-29, or any fragment thereof, or is encoded by a polynucleotide comprising a sequence having at least 90% or greater identity to a sequence selected from SEQ ID NOs: 30-39, or any fragment thereof.

Clause 10. The composition of clause 9, wherein the Cas9 protein comprises an amino acid sequence having one, two, three, four, five or more changes selected from amino acid substitutions, insertions, or deletions, relative to a sequence selected from SEQ ID NOs: 26-29, or any fragment thereof, or is encoded by a polynucleotide comprising a sequence having one, two, three, four, five or more changes selected from nucleotide substitutions, insertions, or deletions, relative to a sequence selected from SEQ ID NOs: 30-39, or any fragment thereof.

Clause 11. The composition of clause 9, wherein the Cas9 protein comprises the amino acid sequence of one of SEQ ID NOs: 26-29, or any fragment thereof, or is encoded by a polynucleotide comprising a sequence selected from SEQ ID NOs: 30-39.

Clause 12. The composition of any one of clauses 5-11, wherein the fusion protein comprises more than one second polypeptide domain.

Clause 13. The composition of any one of clauses 5-12, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, MxiI repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

Clause 14. The composition of any one of clauses 5-13, wherein the second polypeptide domain has transcription repression activity.

Clause 15. The composition of clause 14, wherein the second polypeptide domain comprises KRAB.

Clause 16. The composition of clause 15, wherein KRAB comprises an amino acid sequence having at least 90% or greater identity to SEQ ID NO: 45, or any fragment thereof, or is encoded by a polynucleotide comprising a sequence having at least 90% or greater identity to SEQ ID NO: 46, or any fragment thereof.

Clause 17. The composition of clause 15, wherein KRAB comprises an amino acid sequence having one, two, three, four, five or more changes selected from amino acid substitutions, insertions, or deletions, relative to SEQ ID NO: 45, or any fragment thereof, or is encoded by a polynucleotide comprising a sequence having one, two, three, four, five or more changes selected from nucleotide substitutions, insertions, or deletions, relative to SEQ ID NO: 46, or any fragment thereof.

Clause 18. The composition of clause 15, wherein KRAB comprises the amino acid sequence of SEQ ID NO: 45, or any fragment thereof, or is encoded by a polynucleotide comprising the sequence of SEQ ID NO: 46.

Clause 19. The composition of any one of clauses 5-18, wherein the fusion protein comprises an amino acid sequence having at least 90% or greater identity to SEQ ID NO: 47 or 49, or any fragment thereof, or is encoded by a polynucleotide comprising a sequence having at least 90% or greater identity to SEQ ID NO: 48 or 50, or any fragment thereof.

Clause 20. The composition of any one of clauses 5-18, wherein the fusion protein comprises an amino acid sequence having one, two, three, four, five or more changes selected from amino acid substitutions, insertions, or deletions, relative to SEQ ID NO: 47 or 49, or any fragment thereof, or is encoded by a polynucleotide having one, two, three, four, five or more changes selected from nucleotide substitutions, insertions, or deletions, relative to SEQ ID NO: 48 or 50.

Clause 21. The composition of any one of clauses 5-18, wherein the fusion protein comprises the amino acid sequence of SEQ ID NO: 47 or 49, or any fragment thereof, or is encoded by a polynucleotide comprising the sequence of SEQ ID NO: 48 or 50.

Clause 22. The composition of any one of clauses 5-13, wherein the second polypeptide domain has transcription activation activity.

Clause 23. The composition of clause 22, wherein the second polypeptide domain comprises a polypeptide selected from VP16, VP64, p65, TET1, VPR, VPH, Rta, and p300, or a fragment thereof.

Clause 24. The composition of clause 22, wherein the second polypeptide domain comprises VP64, p300, VPH, or VPR, or a fragment thereof.

Clause 25. The composition of clause 23 or 24, wherein the second polypeptide domain comprises an amino acid sequence having at least 90% or greater identity to SEQ ID NO: 41, 42, 53, or 55, or any fragment thereof, or is encoded by a polynucleotide comprising a sequence having at least 90% or greater identity to SEQ ID NO: 54 or 56, or any fragment thereof.

Clause 26. The composition of clause 23 or 24, wherein the second polypeptide domain comprises an amino acid sequence having one, two, three, four, five or more changes selected from amino acid substitutions, insertions, or deletions, relative to SEQ ID NO: 41, 42, 53, or 55, or any fragment thereof, or is encoded by a polynucleotide comprising a sequence having one, two, three, four, five or more changes selected from nucleotide substitutions, insertions, or deletions, relative to SEQ ID NO: 54 or 56, or any fragment thereof.

Clause 27. The composition of clause 23 or 24, wherein the second polypeptide domain comprises the amino acid sequence of SEQ ID NO: 41, 42, 53, or 55, or any fragment thereof, or is encoded by a polynucleotide comprising the sequence of SEQ ID NO: 54 or 56.

Clause 28. The composition of clause 23 or 24, wherein the fusion protein comprises an amino acid sequence having at least 90% or greater identity to SEQ ID NO: 43, or any fragment thereof, or is encoded by a polynucleotide comprising a sequence having at least 90% or greater identity to SEQ ID NO: 44, or any fragment thereof.

Clause 29. The composition of clause 23 or 24, wherein the fusion protein comprises an amino acid sequence having one, two, three, four, five or more changes selected from amino acid substitutions, insertions, or deletions, relative to SEQ ID NO: 43, or any fragment thereof, or is encoded by a polynucleotide comprising a sequence having one, two, three, four, five or more changes selected from nucleotide substitutions, insertions, or deletions, relative to SEQ ID NO: 44.

Clause 30. The composition of clause 23 or 24, wherein the fusion protein comprises the amino acid sequence of SEQ ID NO: 43, or any fragment thereof, or is encoded by a polynucleotide comprising the sequence of SEQ ID NO: 44.

Clause 31. A composition for increasing T cells, the composition comprising an activator of a gene selected from BATF3, EOMES, NR1 D1, and JUN.

Clause 32. The composition of clause 31, wherein the activator comprises a polynucleotide encoding the gene.

Clause 33. A composition for increasing T cells, the composition comprising an inhibitor of a gene selected from BATF, DNMT1, FOXO1, MYB, and BACH2.

Clause 34. The composition of clause 33, wherein the inhibitor comprises a shRNA or siRNA targeting the gene or a fragment thereof.

Clause 35. A composition for increasing T cells, the composition comprising an activator of the BATF3 gene.

Clause 36. The composition of clause 5 wherein the activator comprises a polynucleotide encoding BATF3.

Clause 37. The composition of clause 31 or 35, wherein the activator comprises the DNA targeting composition of any one of clauses 7-13 and 22-30, and wherein the second polypeptide domain has transcription activation activity.

Clause 38. The composition of clause 37, wherein the gene is BATF3 and the gRNA targets the Cas9 protein to or is encoded by a polynucleotide sequence comprising a sequence selected from SEQ ID NOs: 62-65, or wherein the gene is EOMES and the gRNA targets the Cas9 protein to or is encoded by a polynucleotide sequence comprising a sequence selected from SEQ ID NOs: 57-58, or wherein the gene is NR1D1 and the gRNA targets the Cas9 protein to or is encoded by a polynucleotide sequence comprising the sequence of SEQ ID NO: 82, or wherein the gene is JUN and the gRNA targets the Cas9 protein to or is encoded by a polynucleotide sequence comprising the sequence of SEQ ID NO: 79.

Clause 39. The composition of clause 37, wherein the gene is BATF3 and the gRNA comprises a polynucleotide sequence selected from SEQ ID NOs: 94-97, or wherein the gene is EOMES and the gRNA comprises a polynucleotide sequence selected from SEQ ID NOs: 89-90, or wherein the gene is NR1D1 and the gRNA comprises the polynucleotide sequence of SEQ ID NO: 114, or wherein the gene is JUN and the gRNA comprises the polynucleotide sequence of SEQ ID NO: 111.

Clause 40. The composition of clause 33, wherein the inhibitor comprises the DNA targeting composition of any one of clauses 7-13 and 22-30, and wherein the second polypeptide domain has transcription repression activity.

Clause 41. The composition of clause 40, wherein the gene is BATF and the gRNA targets the Cas9 protein to or is encoded by a polynucleotide sequence comprising a sequence selected from SEQ ID NOs: 59-61, or wherein the gene is DNMT1 and the gRNA targets the Cas9 protein to or is encoded by a polynucleotide sequence comprising the sequence of SEQ ID NO: 71, or wherein the gene is FOXO1 and the gRNA targets the Cas9 protein to or is encoded by a polynucleotide sequence comprising the sequence of SEQ ID NO: 72, or wherein the gene is MYB and the gRNA targets the Cas9 protein to or is encoded by a polynucleotide sequence comprising a sequence selected from SEQ ID NOs: 68-69, or wherein the gene is BACH2 and the and the gRNA targets the Cas9 protein to or is encoded by a polynucleotide sequence comprising the sequence of SEQ ID NO: 85.

Clause 42. The composition of clause 41, wherein the gene is BATF and the gRNA comprises a polynucleotide sequence selected from SEQ ID NOs: 91-93, or wherein the gene is DNMT1 and the gRNA comprises the polynucleotide sequence of SEQ ID NO: 103, or wherein the gene is FOXO1 and the gRNA comprises the polynucleotide sequence of SEQ ID NO: 104, or wherein the gene is MYB and the gRNA comprises a polynucleotide sequence selected from SEQ ID NOs: 100-101, or wherein the gene is BACH2 and the gRNA comprises the polynucleotide sequence of SEQ ID NO: 117.

Clause 43. The composition of any one of clauses 1-43, further comprising at least one cancer therapy.

Clause 44. An isolated polynucleotide sequence encoding the composition of any one of clauses 1-43.

Clause 45. A vector comprising: the isolated polynucleotide sequence of clause 44.

Clause 46. A cell comprising: the composition of any one of clauses 1-43, or the isolated polynucleotide sequence of clause 44, or the vector of clause 45, or a combination thereof.

Clause 47. The cell of clause 46, wherein the cell is a CD8+ T cell.

Clause 48. A pharmaceutical composition comprising: the composition of any one of clauses 1-43, or the isolated polynucleotide sequence of clause 44, or the vector of clause 45, or a combination thereof.

Clause 49. A method of modulating T cells, the method comprising administering to a cell or a subject the composition of any one of clauses 1-43, or the isolated polynucleotide sequence of clause 44, or the vector of clause 45, or the cell of clause 46 or 47, or the pharmaceutical composition of clause 48, or a combination thereof.

Clause 50. The method of clause 49, wherein modulating T cells comprises increasing T cells, or increasing memory T cells, or preventing T cell exhaustions, or reversing T cell exhaustions, or a combination thereof.

Clause 51. A method of increasing T cells, the method comprising administering to a cell or a subject the composition of any one of clauses 1-43, or the isolated polynucleotide sequence of clause 44, or the vector of clause 45, or the cell of clause 46 or 47, or the pharmaceutical composition of clause 48, or a combination thereof.

Clause 52. A method of enhancing adoptive T cell therapy (ACT) in a subject, the method comprising administering to the subject the composition of any one of clauses 1-43, or the isolated polynucleotide sequence of clause 44, or the vector of clause 45, or the cell of clause 46 or 47, or the pharmaceutical composition of clause 48, or a combination thereof.

Clause 53. A method of treating cancer in a subject, the method comprising administering to the subject the composition of any one of clauses 1-43, or the isolated polynucleotide sequence of clause 44, or the vector of clause 45, or the cell of clause 46 or 47, or the pharmaceutical composition of clause 48, or a combination thereof.

SEQUENCES

SEQ ID NO: 1
NRG (R = A or G; N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue, e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; N can be any nucleotide residue, e.g., any of A, G, C, or T;
V = A or C or G)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
SEQ ID NO: 18
DNA sequence of the gRNA constant region
gtttaagagctatgctggaaacagcatagcaagtttaaataaggctagtccgttatcaacttgaaaaa
gtggcaccgagtcggtgc
SEQ ID NO: 19
RNA sequence of the gRNA constant region
guuuaagagcuaugcuggaaacagcauagcaaguuuaaauaaggcuaguccguuaucaacuugaaaaa
guggcaccgagucggugc
SEQ ID NO: 20
SV40 NLS (Pro-Lys-Lys-Lys-Arg-Lys-Val)
SEQ ID NO: 21
GS linker (Gly-Gly-Gly-Gly-Ser)n, wherein n is an integer between 0 and 10
SEQ ID NO: 22
Gly-Gly-Gly-Gly-Gly
SEQ ID NO: 23
Gly-Gly-Ala-Gly-Gly
SEQ ID NO: 24
Gly-Gly-Gly-Gly-Ser-Ser-Ser
SEQ ID NO: 25
Gly-Gly-Gly-Gly-Ala-Ala-Ala
SEQ ID NO: 26
Streptococcus pyogenes Cas9
MDKKYSIGLDIGTNSVGWAVITDEYKVPSKKFKVLGNTDRHSIKKNLIGALLEDSGETAEATRLKRTA
RRRYTRRKNRICYLQEIFSNEMAKVDDSFFHRLEESELVEEDKKHERHPIFGNIVDEVAYHEKYPTIY
HLRKKLVDSTDKADLRLIYLALAHMIKERGHFLIEGDLNPDNSDVDKLFIQLVQTYNQLFEENPINAS
GVDAKAILSARLSKSRRLENLIAQLPGEKKNGLFGNLIALSLGLTPNFKSNEDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKORTEDNG
SIPHQIHLGELHAILRRQEDFYPELKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLEKTNRKVTVKOLKEDYFKKIECFDSVEISGVEDRENASLGTYHDLLKIIKDKDELDNEEN
EDILEDIVLTLTLFEDREMIEERLKTYAHLEDDKVMKOLKRRRYTGWGRLSRKLINGIRDKQSGKTIL
DELKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDERKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 27
Staphylococcus aureus Cas9
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHOLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRESVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 28
Streptococcus pyogenes Cas9 (with D10A)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESELVEEDKKHERHPIFGNIVDEVAYHEKYPTIY
HLRKKLVDSTDKADLRLIYLALAHMIKERGHFLIEGDLNPDNSDVDKLFIQLVQTYNQLFEENPINAS
GVDAKAILSARLSKSRRLENLIAQLPGEKKNGLFGNLIALSLGLTPNFKSNEDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKORTEDNG
SIPHQIHLGELHAILRRQEDFYPFLKDNREKIEKILTFRIPYYVGPLARGNSRFAWMTRKSEETITPW
NFEEVVDKGASAQSFIERMTNEDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDELDNEEN
EDILEDIVLTLTLFEDREMIEERLKTYAHLFDDKVMKOLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDERKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 29
Streptococcus pyogenes Cas9 (with D10A, H849A)
MDKKYSIGLAIGTNSVGWAVITDEYKVPSKKFKVLGNTDRHSIKKNLIGALLEDSGETAEATRLKRTA
RRRYTRRKNRICYLQEIFSNEMAKVDDSFFHRLEESELVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNEKSNEDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKORTEDNG
SIPHQIHLGELHAILRRQEDFYPELKDNREKIEKILTFRIPYYVGPLARGNSRFAWMTRKSEETITPW
NFEEVVDKGASAQSFIERMTNEDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDELDNEEN
EDILEDIVLTLTLFEDREMIEERLKTYAHLFDDKVMKOLKRRRYTGWGRLSRKLINGIRDKQSGKTIL
DELKSDGFANRNEMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDERKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGESKESILPKRNSDKLIARKKDWDPKKYGGEDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYEDTTIDRKRYTSTKEVLDATLIHQSITGLYETRI
DLSQLGGD
SEQ ID NO: 30
Polynucleotide sequence of D10A mutant S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca totttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 31
Polynucleotide sequence of N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagato
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca totttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 32
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tottgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
SEQ ID NO: 33
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca totttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 34
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tottcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat togacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 35
codon optimized nucleic acid sequences encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tottcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
SEQ ID NO: 36
codon optimized nucleic acid sequence encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 37
codon optimized nucleic acid sequence encoding S. aureus Cas9
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
SEQ ID NO: 38
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 39
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 40
Vector (pDO242) encoding codon optimized nucleic acid
sequence encoding S. aureus Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 41
Human p300 (with L553M mutation) protein
MAENVVEPGPPSAKRPKLSSPALSASASDGTDFGSLEDLEHDLPDELINSTELGLINGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCORREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKONMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMENNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGFVCDGCLKKSARTRKENKFSAKR
LPSTRLGTFLENRVNDELRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKOKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETORQMAHVOIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDONSMLSQLASNP
GMANLHGASATDLGLSTDNSDLNSNLSQSTLDIH
SEQ ID NO: 42
Human p300 Core Effector protein (aa 1048-1664 of SEQ ID NO: 41)
IFKPEELRQALMPTLEALYRQDPESLPFRQPVDPQLLGIPDYFDIVKSPMDLSTIKRKLDTGQYQEPW
QYVDDIWLMENNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDELRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDEWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 43
VP64-dCas9-VP64 protein
RADALDDFDLDMLGSDALDDEDLDMLGSDALDDEDLDMLGSDALDDEDLDMVNPKKKRKVGRGMDKKY
SIGLAIGTNSVGWAVITDEYKVPSKKFKVLGNTDRHSIKKNLIGALLEDSGETAEATRLKRTARRRYT
RRKNRICYLQEIFSNEMAKVDDSFFHRLEESELVEEDKKHERHPIFGNIVDEVAYHEKYPTIYHLRKK
LVDSTDKADLRLIYLALAHMIKERGHFLIEGDLNPDNSDVDKLFIQLVQTYNQLFEENPINASGVDAK
AILSARLSKSRRLENLIAQLPGEKKNGLFGNLIALSLGLTPNFKSNEDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKORTEDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRENASLGTYHDLLKIIKDKDELDNEENEDILE
DIVLTLTLFEDREMIEERLKTYAHLEDDKVMKOLKRRRYTGWGRLSRKLINGIRDKQSGKTILDELKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDERKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDEDLDMLGSDALDDEDLDML
I
SEQ ID NO: 44
VP64-dCas9-VP64 DNA
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 45
Polypeptide sequence of KRAB protein
RTLVTFKDVFVDFTREEWKLLDTAQQILYRNVMLENYKNLVSLGYQLTKPDVILRLEKGEEP
WLV
SEQ ID NO: 46
Polynucleotide sequence for KRAB
cggacactggtgaccttcaaggatgtgtttgtggacttcaccagggaggagtggaagctgct
ggacactgctcagcagatcctgtacagaaatgtgatgctggagaactataagaacctggttt
ccttgggttatcagcttactaagccagatgtgatcctccggttggagaagggagaagagccc
tggctggtg
SEQ ID NO: 47
Polypeptide sequence of Streptococcus pyogenes dCas9-KRAB protein
MDYKDHDGDYKDHDIDYKDDDDKMAPKKKRKVGRGMDKKYSIGLAIGTNSVGWAVITDEYKVPSKKEK
VLGNTDRHSIKKNLIGALLFDSGETAEATRLKRTARRRYTRRKNRICYLQEIFSNEMAKVDDSFFHRL
EESELVEEDKKHERHPIFGNIVDEVAYHEKYPTIYHLRKKLVDSTDKADLRLIYLALAHMIKERGHEL
IEGDLNPDNSDVDKLFIQLVQTYNQLFEENPINASGVDAKAILSARLSKSRRLENLIAQLPGEKKNGL
FGNLIALSLGLTPNEKSNEDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKORTEDNGSIPHQIHLGELHAILRRQEDFYPELKDNREKIE
KILTFRIPYYVGPLARGNSRFAWMTRKSEETITPWNFEEVVDKGASAQSFIERMTNEDKNLPNEKVLP
KHSLLYEYFTVYNELTKVKYVTEGMRKPAFLSGEQKKAIVDLLEKTNRKVTVKOLKEDYFKKIECFDS
VEISGVEDRENASLGTYHDLLKIIKDKDELDNEENEDILEDIVLTLTLFEDREMIEERLKTYAHLEDD
KVMKQLKRRRYTGWGRLSRKLINGIRDKQSGKTILDELKSDGFANRNEMQLIHDDSLTFKEDIQKAQV
SGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSELKD
DSIDNKVLTRSDKNRGKSDNVPSEEVVKKMKNYWRQLLNAKLITQRKEDNLTKAERGGLSELDKAGFI
KRQLVETRQITKHVAQILDSRMNTKYDENDKLIREVKVITLKSKLVSDERKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDELEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSRADPKKKRKVASDAKSLTAWSRTL
VTFKDVFVDFTREEWKLLDTAQQILYRNVMLENYKNLVSLGYQLTKPDVILRLEKGEEPWLVEREIHQ
ETHPDSETAFEIKSSVPKKKRKV
SEQ ID NO: 48
Polynucleotide sequence encoding Streptococcus pyogenes dCas9-KRAB
atggactacaaagaccatgacggtgattataaagatcatgacatcgattacaaggatgacgatgacaa
gatggcccccaagaagaagaggaaggtgggccgcggaatggacaagaagtactccattgggctcgcca
tcggcacaaacagcgtcggctgggccgtcattacggacgagtacaaggtgccgagcaaaaaattcaaa
gttctgggcaataccgatcgccacagcataaagaagaacctcattggcgccctcctgttcgactccgg
ggaaaccgccgaagccacgcggctcaaaagaacagcacggcgcagatatacccgcagaaagaatcgga
tctgctacctgcaggagatctttagtaatgagatggctaaggtggatgactctttcttccataggctg
gaggagtcctttttggtggaggaggataaaaagcacgagcgccacccaatctttggcaatatcgtgga
cgaggtggcgtaccatgaaaagtacccaaccatatatcatctgaggaagaagcttgtagacagtactg
ataaggctgacttgcggttgatctatctcgcgctggcgcatatgatcaaatttcggggacacttcctc
atcgagggggacctgaacccagacaacagcgatgtcgacaaactctttatccaactggttcagactta
caatcagcttttcgaagagaacccgatcaacgcatccggagttgacgccaaagcaatcctgagcgcta
ggctgtccaaatcccggcggctcgaaaacctcatcgcacagctccctggggagaagaagaacggcctg
tttggtaatcttatcgccctgtcactcgggctgacccccaactttaaatctaacttcgacctggccga
agatgccaagcttcaactgagcaaagacacctacgatgatgatctcgacaatctgctggcccagatcg
gcgaccagtacgcagacctttttttggcggcaaagaacctgtcagacgccattctgctgagtgatatt
ctgcgagtgaacacggagatcaccaaagctccgctgagcgctagtatgatcaagcgctatgatgagca
ccaccaagacttgactttgctgaaggcccttgtcagacagcaactgcctgagaagtacaaggaaattt
tcttcgatcagtctaaaaatggctacgccggatacattgacggcggagcaagccaggaggaattttac
aaatttattaagcccatcttggaaaaaatggacggcaccgaggagctgctggtaaagcttaacagaga
agatctgttgcgcaaacagcgcactttcgacaatggaagcatcccccaccagattcacctgggcgaac
tgcacgctatcctcaggcggcaagaggatttctacccctttttgaaagataacagggaaaagattgag
aaaatcctcacatttcggataccctactatgtaggccccctcgcccggggaaattccagattcgcgtg
gatgactcgcaaatcagaagagaccatcactccctggaacttcgaggaagtcgtggataagggggcct
ctgcccagtccttcatcgaaaggatgactaactttgataaaaatctgcctaacgaaaaggtgcttcct
aaacactctctgctgtacgagtacttcacagtttataacgagctcaccaaggtcaaatacgtcacaga
agggatgagaaagccagcattcctgtctggagagcagaagaaagctatcgtggacctcctcttcaaga
cgaaccggaaagttaccgtgaaacagctcaaagaagactatttcaaaaagattgaatgtttcgactct
gttgaaatcagcggagtggaggatcgcttcaacgcatccctgggaacgtatcacgatctcctgaaaat
cattaaagacaaggacttcctggacaatgaggagaacgaggacattcttgaggacattgtcctcaccc
ttacgttgtttgaagatagggagatgattgaagaacgcttgaaaacttacgctcatctcttcgacgac
aaagtcatgaaacagctcaagaggcgccgatatacaggatgggggcggctgtcaagaaaactgatcaa
tgggatccgagacaagcagagtggaaagacaatcctggattttcttaagtccgatggatttgccaacc
ggaacttcatgcagttgatccatgatgactctctcacctttaaggaggacatccagaaagcacaagtt
tctggccagggggacagtcttcacgagcacatcgctaatcttgcaggtagcccagctatcaaaaaggg
aatactgcagaccgttaaggtcgtggatgaactcgtcaaagtaatgggaaggcataagcccgagaata
tcgttatcgagatggcccgagagaaccaaactacccagaagggacagaagaacagtagggaaaggatg
aagaggattgaagagggtataaaagaactggggtcccaaatccttaaggaacacccagttgaaaacac
ccagcttcagaatgagaagctctacctgtactacctgcagaacggcagggacatgtacgtggatcagg
aactggacatcaatcggctctccgactacgacgtggatgccatcgtgccccagtcttttctcaaagat
gattctattgataataaagtgttgacaagatccgataaaaatagagggaagagtgataacgtcccctc
agaagaagttgtcaagaaaatgaaaaattattggcggcagctgctgaacgccaaactgatcacacaac
ggaagttcgataatctgactaaggctgaacgaggtggcctgtctgagttggataaagccggcttcatc
aaaaggcagcttgttgagacacgccagatcaccaagcacgtggcccaaattctcgattcacgcatgaa
caccaagtacgatgaaaatgacaaactgattcgagaggtgaaagttattactctgaagtctaagctgg
tctcagatttcagaaaggactttcagttttataaggtgagagagatcaacaattaccaccatgcgcat
gatgcctacctgaatgcagtggtaggcactgcacttatcaaaaaatatcccaagcttgaatctgaatt
tgtttacggagactataaagtgtacgatgttaggaaaatgatcgcaaagtctgagcaggaaataggca
aggccaccgctaagtacttcttttacagcaatattatgaattttttcaagaccgagattacactggcc
aatggagagattcggaagcgaccacttatcgaaacaaacggagaaacaggagaaatcgtgtgggacaa
gggtagggatttcgcgacagtccggaaggtcctgtccatgccgcaggtgaacatcgttaaaaagaccg
aagtacagaccggaggcttctccaaggaaagtatcctcccgaaaaggaacagcgacaagctgatcgca
cgcaaaaaagattgggaccccaagaaatacggcggattcgattctcctacagtcgcttacagtgtact
ggttgtggccaaagtggagaaagggaagtctaaaaaactcaaaagcgtcaaggaactgctgggcatca
caatcatggagcgatcaagcttcgaaaaaaaccccatcgactttctcgaggcgaaaggatataaagag
gtcaaaaaagacctcatcattaagcttcccaagtactctctctttgagcttgaaaacggccggaaacg
aatgctcgctagtgcgggcgagctgcagaaaggtaacgagctggcactgccctctaaatacgttaatt
tcttgtatctggccagccactatgaaaagctcaaagggtctcccgaagataatgagcagaagcagctg
ttcgtggaacaacacaaacactaccttgatgagatcatcgagcaaataagcgaattctccaaaagagt
gatcctcgccgacgctaacctcgataaggtgctttctgcttacaataagcacagggataagcccatca
gggagcaggcagaaaacattatccacttgtttactctgaccaacttgggcgcgcctgcagccttcaag
tacttcgacaccaccatagacagaaagcggtacacctctacaaaggaggtcctggacgccacactgat
tcatcagtcaattacggggctctatgaaacaagaatcgacctctctcagctcggtggagacagcaggg
ctgaccccaagaagaagaggaaggtggctagcgatgctaagtcactgactgcctggtcccggacactg
gtgaccttcaaggatgtgtttgtggacttcaccagggaggagtggaagctgctggacactgctcagca
gatcctgtacagaaatgtgatgctggagaactataagaacctggtttccttgggttatcagcttacta
agccagatgtgatcctccggttggagaagggagaagagccctggctggtggagagagaaattcaccaa
gagacccatcctgattcagagactgcatttgaaatcaaatcatcagttccgaaaaagaaacgcaaagt
ttga
SEQ ID NO: 49
Polypeptide sequence of Staphylococcus aureus dCas9-KRAB protein
MAPKKKRKVGIHGVPAAKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYAYNADLY
NALNDLNNLVITRDENEKLEYYEKFQIIENVFKQKKKPTLKQIAKEILVNEEDIKGYRVTSTGKPEFT
NLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKV
INAIIKKYGLPNDIIIELAREKNSKDAQKMINEMQKRNRQTNERIEEIIRTTGKENAKYLIEKIKLHD
MQEGKCLYSLEAIPLEDLLNNPFNYEVDHIIPRSVSFDNSFNNKVLVKQEEASKKGNRTPFQYLSSSD
SKISYETFKKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLDKAKKVMENQM
FEEKQAESMPEIETEQEYKEIFITPHQIKHIKDEKDYKYSHRVDKKPNRELINDTLYSTRKDDKGNTL
IVNNLNGLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYNNDLIKINGELYRVIGVNNDLLNRIEVNMIDITYREY
LENMNDKRPPRIIKTIASKTQSIKKYSTDILGNLYEVKSKKHPQIIKKGKRPAATKKAGQAKKKKGSD
AKSLTAWSRTLVTFKDVFVDFTREEWKLLDTAQQILYRNVMLENYKNLVSLGYQLTKPDVILRLEKGE
EPWLVEREIHQETHPDSETAFEIKSSVPKKKRKV
SEQ ID NO: 50
Polynucleotide sequence of Staphylococcus aureus dCas9-KRAB protein
atggccccaaagaagaagcggaaggtcggtatccacggagtcccagcagccaagcggaactacatcct
gggcctggccatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaagccagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaagggatccgat
gctaagtcactgactgcctggtcccggacactggtgaccttcaaggatgtgtttgtggacttcaccag
ggaggagtggaagctgctggacactgctcagcagatcctgtacagaaatgtgatgctggagaactata
agaacctggtttccttgggttatcagcttactaagccagatgtgatcctccggttggagaagggagaa
gagccctggctggtggagagagaaattcaccaagagacccatcctgattcagagactgcatttgaaat
caaatcatcagttccgaaaaagaaacgcaaagtt
SEQ ID NO: 51
Polypeptide sequence of Tet1CD
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRTCTCQGIDPETCGASESFGCSWSMYFNGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPQDEQLHVLPLYKLSDTDEFGSKEGMEAKIKSGAIEVLAPRRKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSELTSPODLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
SEQ ID NO: 52
Polynucleotide sequence of Tet1CD
CTGCCCACCTGCAGCTGTCTTGATCGAGTTATACAAAAAGACAAAGGCCCATATTATACACACCTTGG
GGCAGGACCAAGTGTTGCTGCTGTCAGGGAAATCATGGAGAATAGGTATGGTCAAAAAGGAAACGCAA
TAAGGATAGAAATAGTAGTGTACACCGGTAAAGAAGGGAAAAGCTCTCATGGGTGTCCAATTGCTAAG
TGGGTTTTAAGAAGAAGCAGTGATGAAGAAAAAGTTCTTTGTTTGGTCCGGCAGCGTACAGGCCACCA
CTGTCCAACTGCTGTGATGGTGGTGCTCATCATGGTGTGGGATGGCATCCCTCTTCCAATGGCCGACC
GGCTATACACAGAGCTCACAGAGAATCTAAAGTCATACAATGGGCACCCTACCGACAGAAGATGCACC
CTCAATGAAAATCGTACCTGTACATGTCAAGGAATTGATCCAGAGACTTGTGGAGCTTCATTCTCTTT
TGGCTGTTCATGGAGTATGTACTTTAATGGCTGTAAGTTTGGTAGAAGCCCAAGCCCCAGAAGATTTA
GAATTGATCCAAGCTCTCCCTTACATGAAAAAAACCTTGAAGATAACTTACAGAGTTTGGCTACACGA
TTAGCTCCAATTTATAAGCAGTATGCTCCAGTAGCTTACCAAAATCAGGTGGAATATGAAAATGTTGC
CCGAGAATGTCGGCTTGGCAGCAAGGAAGGTCGACCCTTCTCTGGGGTCACTGCTTGCCTGGACTTCT
GTGCTCATCCCCACAGGGACATTCACAACATGAATAATGGAAGCACTGTGGTTTGTACCTTAACTCGA
GAAGATAACCGCTCTTTGGGTGTTATTCCTCAAGATGAGCAGCTCCATGTGCTACCTCTTTATAAGCT
TTCAGACACAGATGAGTTTGGCTCCAAGGAAGGAATGGAAGCCAAGATCAAATCTGGGGCCATCGAGG
TCCTGGCACCCCGCCGCAAAAAAAGAACGTGTTTCACTCAGCCTGTTCCCCGTTCTGGAAAGAAGAGG
GCTGCGATGATGACAGAGGTTCTTGCACATAAGATAAGGGCAGTGGAAAAGAAACCTATTCCCCGAAT
CAAGCGGAAGAATAACTCAACAACAACAAACAACAGTAAGCCTTCGTCACTGCCAACCTTAGGGAGTA
ACACTGAGACCGTGCAACCTGAAGTAAAAAGTGAAACCGAACCCCATTTTATCTTAAAAAGTTCAGAC
AACACTAAAACTTATTCGCTGATGCCATCCGCTCCTCACCCAGTGAAAGAGGCATCTCCAGGCTTCTC
CTGGTCCCCGAAGACTGCTTCAGCCACACCAGCTCCACTGAAGAATGACGCAACAGCCTCATGCGGGT
TTTCAGAAAGAAGCAGCACTCCCCACTGTACGATGCCTTCGGGAAGACTCAGTGGTGCCAATGCTGCA
GCTGCTGATGGCCCTGGCATTTCACAGCTTGGCGAAGTGGCTCCTCTCCCCACCCTGTCTGCTCCTGT
GATGGAGCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGATGAGCAG
CATTCTGAAGCAGATGAGCCTCCATCAGACGAACCCCTATCTGATGACCCCCTGTCACCTGCTGAGGA
GAAATTGCCCCACATTGATGAGTATTGGTCAGACAGTGAGCACATCTTTTTGGATGCAAATATTGGTG
GGGTGGCCATCGCACCTGCTCACGGCTCGGTTTTGATTGAGTGTGCCCGGCGAGAGCTGCACGCTACC
ACTCCTGTTGAGCACCCCAACCGTAATCATCCAACCCGCCTCTCCCTTGTCTTTTACCAGCACAAAAA
CCTAAATAAGCCCCAACATGGTTTTGAACTAAACAAGATTAAGTTTGAGGCTAAAGAAGCTAAGAATA
AGAAAATGAAGGCCTCAGAGCAAAAAGACCAGGCAGCTAATGAAGGTCCAGAACAGTCCTCTGAAGTA
AATGAATTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
SEQ ID NO: 53
Protein sequence for VPH
DALDDFDLDMLGSDALDDEDLDMLGSDALDDFDLDMLGSDALDDFDLDMLGSLPSASVEFEGSGGPSG
QISNQALALAPSSAPVLAQTMVPSSAMVPLAQPPAPAPVLTPGPPQSLSAPVPKSTQAGEGTLSEALL
HLQFDADEDLGALLGNSTDPGVFTDLASVDNSEFQQLLNQGVSMSHSTAEPMLMEYPEAITRLVTGSQ
RPPDPAPTPLGTSGLPNGLSGDEDESSIADMDFSALLSQISSSGQGGGGSGFSVDTSALLDLFSPSVT
VPDMSLPDLDSSLASIQELLSPQEPPRPPEAENSSPDSGKQLVHYTAQPLFLLDPGSVDTGSNDLPVL
FELGEGSYFSEGDGFAEDPTISLLTGSEPPKAKDPTVS
SEQ ID NO: 54
DNA sequence for VPH
Gatgctttagacgattttgacttagatatgcttggttcagacgcgttagacgacttcgacctagacat
gttaggctcagatgcattggacgacttcgatttagatatgttgggctccgatgccctagatgactttg
atctagatatgctagggtcactacccagcgccagcgtcgagttcgaaggcagcggcgggccttcaggg
cagatcagcaaccaggccctggctctggcccctagctccgctccagtgctggcccagactatggtgcc
ctctagtgctatggtgcctctggcccagccacctgctccagcccctgtgctgaccccaggaccacccc
agtcactgagcgccccagtgcccaagtctacacaggccggcgaggggactctgagtgaagctctgctg
cacctgcagttcgacgctgatgaggacctgggagctctgctggggaacagcaccgatcccggagtgtt
cacagatctggcctccgtggacaactctgagtttcagcagctgctgaatcagggcgtgtccatgtctc
atagtacagccgaaccaatgctgatggagtaccccgaagccattacccggctggtgaccggcagccag
cggccccccgaccccgctccaactcccctgggaaccagcggcctgcctaatgggctgtccggagatga
agacttctcaagcatcgctgatatggactttagtgccctgctgtcacagatttcctctagtgggcagg
gaggaggtggaagcggcttcagcgtggacaccagtgccctgctggacctgttcagcccctcggtgacc
gtgcccgacatgagcctgcctgaccttgacagcagcctggccagtatccaagagctcctgtctcccca
ggagccccccaggcctcccgaggcagagaacagcagcccggattcagggaagcagctggtgcactaca
cagcgcagccgctgttcctgctggaccccggctccgtggacaccgggagcaacgacctgccggtgctg
tttgagctgggagagggctcctacttctccgaaggggacggcttcgccgaggaccccaccatctccct
gctgacaggctcggagcctcccaaagccaaggaccccactgtctcc
SEQ ID NO: 55
Protein sequence for VPR
DALDDFDLDMLGSDALDDFDLDMLGSDALDDEDLDMLGSDALDDEDLDMLGSPKKKRKVGSQYLPDTD
DRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKPT
QAGEGTLSEALLQLQFDDEDLGALLGNSTDPAVFTDLASVDNSEFQQLLNQGIPVAPHTTEPMLMEYP
EAITRLVTGAQRPPDPAPAPLGAPGLPNGLLSGDEDESSIADMDESALLSQISSGSGSGSRDSREGME
LPKPEAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTTTLES
MTEDLNLDSPLTPELNEILDTELNDECLLHAMHISTGLSIFDTSLF
SEQ ID NO: 56
DNA sequence for VPR
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctaggtagtcccaaaaagaagaggaaagtgggatcccagtatctgcccgacacagat
gatagacaccgaatcgaagagaaacgcaagcgaacgtatgaaaccttcaaatcgatcatgaagaaatc
gcccttctcgggtccgaccgatcccaggcccccaccgagaaggattgcggtcccgtcccgctcgtcgg
ccagcgtgccgaagcctgcgccgcagccctaccccttcacgtcgagcctgagcacaatcaattatgac
gagttcccgacgatggtgttcccctcgggacaaatctcacaagcctcggcgctcgcaccagcgcctcc
ccaagtccttccgcaagcgcctgccccagcgcctgcaccggcaatggtgtccgccctcgcacaggccc
ctgcgcccgtccccgtgctcgcgcctggaccgccccaggcggtcgctccaccggctccgaagccgacg
caggccggagagggaacactctccgaagcacttcttcaactccagtttgatgacgaggatcttggagc
actccttggaaactcgacagaccctgcggtgtttaccgacctcgcgtcagtagataactccgaatttc
agcagcttttgaaccagggtatcccggtcgcgccacatacaacggagcccatgttgatggaatacccc
gaagcaatcacgagacttgtgacgggagcgcagcggcctcccgatcccgcacccgcacctttgggggc
acctggcctccctaacggacttttgagcggcgacgaggatttctcctccatcgccgatatggatttct
cagccttgctgtcacagatttccagcggctctggcagcggcagccgggattccagggaagggatgttt
ttgccgaagcctgaggccggctccgctattagtgacgtgtttgagggccgcgaggtgtgccagccaaa
acgaatccggccatttcatcctccaggaagtccatgggccaaccgcccactccccgccagcctcgcac
caacaccaaccggtccagtacatgagccagtcgggtcactgaccccggcaccagtccctcagccactg
gatccagcgcccgcagtgactcccgaggccagtcacctgttggaggatcccgatgaagagacgagcca
ggctgtcaaagcccttcgggagatggccgatactgtgattccccagaaggaagaggctgcaatctgtg
gccaaatggacctttcccatccgcccccaaggggccatctggatgagctgacaaccacacttgagtcc
atgaccgaggatctgaacctggactcacccctgaccccggaattgaacgagattctggataccttcct
gaacgacgagtgcctcttgcatgccatgcatatcagcacaggactgtccatcttcgacacatctctgt
tt

1. A composition for modulating T cells, the composition comprising a modulator of a gene selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and F11.

2. The composition of claim 1, wherein modulating T cells comprises increasing T cells, or increasing memory T cells, or increasing the lifetime of a T cell, or preventing T cell exhaustions, or reversing T cell exhaustions, or reducing T cell exhaustion, or enhancing the therapeutic potential of T cells, or a combination thereof.

3. The composition of claim 1 or 2, wherein the modulator comprises a polypeptide, or a polynucleotide, or a small molecule, or a lipid, or a carbohydrate, or a combination thereof.

4. The composition of claim 3, wherein the modulator comprises an antibody or siRNA or shRNA.

5. The composition of claim 3, wherein the modulator comprises a DNA targeting composition, the DNA targeting composition comprising:
a Cas9 protein or a fusion protein, wherein the fusion protein comprises two heterologous polypeptide domains, wherein the first polypeptide domain comprises a Cas9 protein and the second polypeptide domain has an activity selected from transcription activation activity, transcription repression activity, nuclease activity, transcription release factor activity, histone modification activity, nucleic acid association activity, methylase activity, and demethylase activity; and
at least one guide RNA (gRNA) that targets the Cas9 protein to the gene or a regulatory element thereof.

6. A DNA targeting composition comprising:
a Cas9 protein or a fusion protein, wherein the fusion protein comprises two heterologous polypeptide domains, wherein the first polypeptide domain comprises a Cas9 protein and the second polypeptide domain has an activity selected from transcription activation activity, transcription repression activity, nuclease activity, transcription release factor activity, histone modification activity, nucleic acid association activity, methylase activity, and demethylase activity; and
at least one guide RNA (gRNA) that targets the Cas9 protein to a target gene or a regulatory element thereof, wherein the target gene is selected from BATF3, BATF, EOMES, BHLHE40, CREM, NFE2L1, NR1D1, POU2F1, FOXD2, GABPA, RREB1, JUN, ZFP1, IRF2, NFATC3, NR4A1, DNMT1, FOXO1, MYB, TCF7L1, BACH2, HIC1, KLF2, and FLI1.

7. The composition of any one of claims 5-6, wherein the gRNA is encoded by a polynucleotide comprising a sequence selected from SEQ ID NOs: 57-88, or comprises a sequence selected from SEQ ID NOs: 89-120.

8. The composition of any one of claims 5-7, wherein the Cas protein comprises a Streptococcus pyogenes Cas9 protein, or a Staphylococcus aureus Cas9 protein, or any fragment thereof.

9. The composition of any one of claims 5-8, wherein the Cas9 protein comprises an amino acid sequence having at least 90% or greater identity to a sequence selected from SEQ ID NOs: 26-29, or any fragment thereof, or is encoded by a polynucleotide comprising a sequence having at least 90% or greater identity to a sequence selected from SEQ ID NOs: 30-39, or any fragment thereof.

10. The composition of claim 9, wherein the Cas9 protein comprises an amino acid sequence having one, two, three, four, five or more changes selected from amino acid substitutions, insertions, or deletions, relative to a sequence selected from SEQ ID NOs: 26-29, or any fragment thereof, or is encoded by a polynucleotide comprising a sequence having one, two, three, four, five or more changes selected from nucleotide substitutions, insertions, or deletions, relative to a sequence selected from SEQ ID NOs: 30-39, or any fragment thereof.

11. The composition of claim 9, wherein the Cas9 protein comprises the amino acid sequence of one of SEQ ID NOs: 26-29, or any fragment thereof, or is encoded by a polynucleotide comprising a sequence selected from SEQ ID NOs: 30-39.

12. The composition of any one of claims 5-11, wherein the fusion protein comprises more than one second polypeptide domain.

13. The composition of any one of claims 5-12, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, MxiI repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

14. The composition of any one of claims 5-13, wherein the second polypeptide domain has transcription repression activity.

15. The composition of claim 14, wherein the second polypeptide domain comprises KRAB.

16. The composition of claim 15, wherein KRAB comprises an amino acid sequence having at least 90% or greater identity to SEQ ID NO: 45, or any fragment thereof, or is encoded by a polynucleotide comprising a sequence having at least 90% or greater identity to SEQ ID NO: 46, or any fragment thereof.

17. The composition of claim 15, wherein KRAB comprises an amino acid sequence having one, two, three, four, five or more changes selected from amino acid substitutions, insertions, or deletions, relative to SEQ ID NO: 45, or any fragment thereof, or is encoded by a polynucleotide comprising a sequence having one, two, three, four, five or more changes selected from nucleotide substitutions, insertions, or deletions, relative to SEQ ID NO: 46, or any fragment thereof.

18. The composition of claim 15, wherein KRAB comprises the amino acid sequence of SEQ ID NO: 45, or any fragment thereof, or is encoded by a polynucleotide comprising the sequence of SEQ ID NO: 46.

19. The composition of any one of claims 5-18, wherein the fusion protein comprises an amino acid sequence having at least 90% or greater identity to SEQ ID NO: 47 or 49, or any fragment thereof, or is encoded by a polynucleotide comprising a sequence having at least 90% or greater identity to SEQ ID NO: 48 or 50, or any fragment thereof.

20. The composition of any one of claims 5-18, wherein the fusion protein comprises an amino acid sequence having one, two, three, four, five or more changes selected from amino acid substitutions, insertions, or deletions, relative to SEQ ID NO: 47 or 49, or any fragment thereof, or is encoded by a polynucleotide having one, two, three, four, five or more changes selected from nucleotide substitutions, insertions, or deletions, relative to SEQ ID NO: 48 or 50.

21. The composition of any one of claims 5-18, wherein the fusion protein comprises the amino acid sequence of SEQ ID NO: 47 or 49, or any fragment thereof, or is encoded by a polynucleotide comprising the sequence of SEQ ID NO: 48 or 50.

22. The composition of any one of claims 5-13, wherein the second polypeptide domain has transcription activation activity.

23. The composition of claim 22, wherein the second polypeptide domain comprises a polypeptide selected from VP16, VP64, p65, TET1, VPR, VPH, Rta, and p300, or a fragment thereof.

24. The composition of claim 22, wherein the second polypeptide domain comprises VP64, p300, VPH, or VPR, or a fragment thereof.

25. The composition of claim 23 or 24, wherein the second polypeptide domain comprises an amino acid sequence having at least 90% or greater identity to SEQ ID NO: 41, 42, 53, or 55, or any fragment thereof, or is encoded by a polynucleotide comprising a sequence having at least 90% or greater identity to SEQ ID NO: 54 or 56, or any fragment thereof.

26. The composition of claim 23 or 24, wherein the second polypeptide domain comprises an amino acid sequence having one, two, three, four, five or more changes selected from amino acid substitutions, insertions, or deletions, relative to SEQ ID NO: 41, 42, 53, or 55, or any fragment thereof, or is encoded by a polynucleotide comprising a sequence having one, two, three, four, five or more changes selected from nucleotide substitutions, insertions, or deletions, relative to SEQ ID NO: 54 or 56, or any fragment thereof.

27. The composition of claim 23 or 24, wherein the second polypeptide domain comprises the amino acid sequence of SEQ ID NO: 41, 42, 53, or 55, or any fragment thereof, or is encoded by a polynucleotide comprising the sequence of SEQ ID NO: 54 or 56.

28. The composition of claim 23 or 24, wherein the fusion protein comprises an amino acid sequence having at least 90% or greater identity to SEQ ID NO: 43, or any fragment thereof, or is encoded by a polynucleotide comprising a sequence having at least 90% or greater identity to SEQ ID NO: 44, or any fragment thereof.

29. The composition of claim 23 or 24, wherein the fusion protein comprises an amino acid sequence having one, two, three, four, five or more changes selected from amino acid substitutions, insertions, or deletions, relative to SEQ ID NO: 43, or any fragment thereof, or is encoded by a polynucleotide comprising a sequence having one, two, three, four, five or more changes selected from nucleotide substitutions, insertions, or deletions, relative to SEQ ID NO: 44.

30. The composition of claim 23 or 24, wherein the fusion protein comprises the amino acid sequence of SEQ ID NO: 43, or any fragment thereof, or is encoded by a polynucleotide comprising the sequence of SEQ ID NO: 44.

31. A composition for increasing T cells, the composition comprising an activator of a gene selected from BATF3, EOMES, NR1D1, and JUN.

32. The composition of claim 31, wherein the activator comprises a polynucleotide encoding the gene.

33. A composition for increasing T cells, the composition comprising an inhibitor of a gene selected from BATF, DNMT1, FOXO1, MYB, and BACH2.

34. The composition of claim 33, wherein the inhibitor comprises a shRNA or siRNA targeting the gene or a fragment thereof.

35. A composition for increasing T cells, the composition comprising an activator of the BATF3 gene.

36. The composition of claim 5 wherein the activator comprises a polynucleotide encoding BATF3.

37. The composition of claim 31 or 35, wherein the activator comprises the DNA targeting composition of any one of claims 7-13 and 22-30, and wherein the second polypeptide domain has transcription activation activity.

38. The composition of claim 37, wherein the gene is BATF3 and the gRNA targets the Cas9 protein to or is encoded by a polynucleotide sequence comprising a sequence selected from SEQ ID NOs: 62-65,
or wherein the gene is EOMES and the gRNA targets the Cas9 protein to or is encoded by a polynucleotide sequence comprising a sequence selected from SEQ ID NOs: 57-58,
or wherein the gene is NR1D1 and the gRNA targets the Cas9 protein to or is encoded by a polynucleotide sequence comprising the sequence of SEQ ID NO: 82,
or wherein the gene is JUN and the gRNA targets the Cas9 protein to or is encoded by a polynucleotide sequence comprising the sequence of SEQ ID NO: 79.

39. The composition of claim 37, wherein the gene is BATF3 and the gRNA comprises a polynucleotide sequence selected from SEQ ID NOs: 94-97,
or wherein the gene is EOMES and the gRNA comprises a polynucleotide sequence selected from SEQ ID NOs: 89-90,
or wherein the gene is NR1D1 and the gRNA comprises the polynucleotide sequence of SEQ ID NO: 114,
or wherein the gene is JUN and the gRNA comprises the polynucleotide sequence of SEQ ID NO: 111.

40. The composition of claim 33, wherein the inhibitor comprises the DNA targeting composition of any one of claims 7-13 and 22-30, and wherein the second polypeptide domain has transcription repression activity.

41. The composition of claim 40, wherein the gene is BATF and the gRNA targets the Cas9 protein to or is encoded by a polynucleotide sequence comprising a sequence selected from SEQ ID NOs: 59-61,
or wherein the gene is DNMT1 and the gRNA targets the Cas9 protein to or is encoded by a polynucleotide sequence comprising the sequence of SEQ ID NO: 71,
or wherein the gene is FOXO1 and the gRNA targets the Cas9 protein to or is encoded by a polynucleotide sequence comprising the sequence of SEQ ID NO: 72,
or wherein the gene is MYB and the gRNA targets the Cas9 protein to or is encoded by a polynucleotide sequence comprising a sequence selected from SEQ ID NOs: 68-69,
or wherein the gene is BACH2 and the and the gRNA targets the Cas9 protein to or is encoded by a polynucleotide sequence comprising the sequence of SEQ ID NO: 85.

42. The composition of claim 41, wherein the gene is BATF and the gRNA comprises a polynucleotide sequence selected from SEQ ID NOs: 91-93,
or wherein the gene is DNMT1 and the gRNA comprises the polynucleotide sequence of SEQ ID NO: 103,
or wherein the gene is FOXO1 and the gRNA comprises the polynucleotide sequence of SEQ ID NO: 104,
or wherein the gene is MYB and the gRNA comprises a polynucleotide sequence selected from SEQ ID NOs: 100-101,
or wherein the gene is BACH2 and the gRNA comprises the polynucleotide sequence of SEQ ID NO: 117.

43. The composition of any one of claims 1-43, further comprising at least one cancer therapy.

44. An isolated polynucleotide sequence encoding the composition of any one of claims 1-43.

45. A vector comprising: the isolated polynucleotide sequence of claim 44.

46. A cell comprising: the composition of any one of claims 1-43, or the isolated polynucleotide sequence of claim 44, or the vector of claim 45, or a combination thereof.

47. The cell of claim 46, wherein the cell is a CD8+ T cell.

48. A pharmaceutical composition comprising: the composition of any one of claims 1-43, or the isolated polynucleotide sequence of claim 44, or the vector of claim 45, or a combination thereof.

49. A method of modulating T cells, the method comprising administering to a cell or a subject the composition of any one of claims 1-43, or the isolated polynucleotide sequence of claim 44, or the vector of claim 45, or the cell of claim 46 or 47, or the pharmaceutical composition of claim 48, or a combination thereof.

50. The method of claim 49, wherein modulating T cells comprises increasing T cells, or increasing memory T cells, or preventing T cell exhaustions, or reversing T cell exhaustions, or a combination thereof.

51. A method of increasing T cells, the method comprising administering to a cell or a subject the composition of any one of claims 1-43, or the isolated polynucleotide sequence of claim 44, or the vector of claim 45, or the cell of claim 46 or 47, or the pharmaceutical composition of claim 48, or a combination thereof.

52. A method of enhancing adoptive T cell therapy (ACT) in a subject, the method comprising administering to the subject the composition of any one of claims 1-43, or the isolated polynucleotide sequence of claim 44, or the vector of claim 45, or the cell of claim 46 or 47, or the pharmaceutical composition of claim 48, or a combination thereof.

53. A method of treating cancer in a subject, the method comprising administering to the subject the composition of any one of claims 1-43, or the isolated polynucleotide sequence of claim 44, or the vector of claim 45, or the cell of claim 46 or 47, or the pharmaceutical composition of claim 48, or a combination thereof.
